(19) United States
(12) Patent Application Publication
(10) Pub. No.: US 2018/0282331 A1
(43) Pub. Date: Oct. 4, 2018

(54) 7-Oxo -6-(sulfooxy)- 1,6-diazabicyclo [3.2.1] octane containing compounds and their use in treatment of bacterial infections (changed by PCT to: 7-OXO -6-(SULFOOXY)- 1,6-DIAZABICYCLO [3.2.1] OCTANE CONTAINING COMPOUNDS AND THEIR USE IN TREATMENT OF BACTERIAL INFECTIONS

(71) Applicant: WOCKHARDT LIMITED, Chikalthana, Aurangabad, (IN)

(72) Inventors: Ravikumar TADIPARTHI, Aurangabad - 6, Maharashtra (IN); Vijaykumar Jagdishwar PATIL, Solapur - 4, Maharashtra (IN); Deepak DEKHANE, Pune - 6, Maharashtra (IN); Mohammad Usman SHAIKH, Ahmednagar Maharashtra, (IN); Satish BIRAJDAR, Maharashtra (IN); Bharat DOND, Aurangabad - 6, Maharashtra (IN); Mahesh Vithalbhai PATEL, Aurongabad 3, Maharashtra (IN)

(73) Assignee: WOCKHARDT LIMITED, Chikalthana, Aurangabad (IN)

(21) Appl. No.: 15/766,183

(22) PCT Filed: Nov. 9, 2016
(86) PCT No.: PCT/IB2016/056732

§ 371 Date: Apr. 5, 2018

(30) Foreign Application Priority Data
Nov. 9, 2015 (IN) 4280/MUM/2015

(51) Int. Cl. C07D 471/08; A61P 31/04

(52) U.S. Cl. CPC C07D 471/08; A61K 31/407; A61K 2300/00; A61P 31/04

(57) ABSTRACT

Compounds of Formula (I) or a stereoisomer or a pharmaceutically acceptable salt thereof, their preparation, and use in treating a bacterial infection are disclosed.

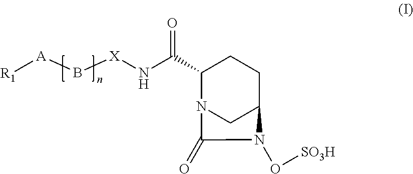

### PRIORITY APPLICATION(S)

This application claims priority to Indian Patent Application No. 4280/MUM/2015 filed on Nov. 9, 2015, the disclosures of which is incorporated herein by reference in its entirety as if fully rewritten herein.

### FIELD OF THE INVENTION

The invention relates to 7-oxo-6-(sulfooxy)-1,6-diazabicyclo[3.2.1]octane containing compounds, their preparation and their use in treating infections.

### BACKGROUND OF INVENTION

Emergence of bacterial resistance to known antibacterial agents is becoming a major challenge in treating bacterial infections. One way forward to treat bacterial infections, and especially those caused by resistant bacteria, is to develop newer antibacterial agents that can overcome the bacterial resistant. Coates et al. (Br. J. Pharmacol. 2007; 152(8), 1147-1154.) have reviewed novel approaches to developing new antibiotics. However, the development of new antibacterial agents is a challenging task. For example, Gwynn et al. (Annals of the New York Academy of Sciences, 2010, 1213: 5-19) have reviewed the challenges in discovery of antibacterial agents.

Several antibacterial agents have been described in the prior art (for example, see PCT International Application Nos. PCT/US2010/060923, PCT/EP2010/067647, PCT/US2010/052109, PCT/US 2010/048109, PCT/GB 2009/050609, PCT/FR01/02418, PCT/EP2009/056178, PCT/US2009/041200, PCT/IB2012/054290, PCT/IB2013/053092, PCT/IB2012/054296, PCT/IB2012/054706, PCT/JP2013/064971, PCT/IB2012/002675, PCT/US2013/034562 and PCT/US2013/034589, PCT/IB2015/050455, PCT/IB2015/050462 and PCT/IB2015/050466). However, there remains a need for development of antibacterial agents for treating bacterial infections, including those caused by bacteria that are resistant to known antibacterial agents.

The inventors have surprisingly discovered novel 7-oxo-6-(sulfooxy)-1,6-diazabicyclo[3.2.1]octane containing compounds having antibacterial activity.

### SUMMARY OF THE INVENTION

Accordingly, there are provided 7-oxo-6-(sulfooxy)-1,6-diazabicyclo[3.2.1[octane containing compounds, methods for preparation of these compounds, pharmaceutical compositions comprising these compounds, and methods for treating a bacterial infection in a subject using these compounds.

In one aspect, there are provided compounds of Formula (I):

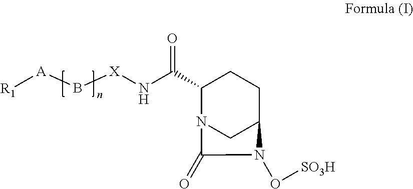

wherein:
A is a four to six membered nitrogen containing ring optionally substituted with one or more substituents selected from C₁-C₆ alkyl, oxo, NR₂R₃, aryl, heteroaryl, cycloalkyl or heterocycloalkyl;
B is C═O or CH—Z;
X is O or CONH;
Z is H, C₁-C₆ alkyl, OR₂ or NR₂₃;
R₁ is selected from:
   (a) hydrogen,
   (b) C₁-C₆ alkyl optionally substituted with one or more substituents independently selected from OR₂, NR₂R₃, SR₂, halogen, CN, COOR₂, CONR₂R₃, SR₂, CH₂OR₂, CH₂NR₂R₃, OCOR₂, aryl, heteroaryl, cycloalkyl or heterocycloalkyl,
   (c) CN,
   (d) CONR₂R₃,
   (e) COOR₂,
   (f) NR₂R₃,
   (g) NHCOOR₂,
   (h) aryl,
   (i) heteroaryl,
   (j) cycloalkyl, or
   (k) heterocycloalkyl;
R₂ and R₃ are each independently selected from:
   (a) hydrogen,
   (b) C₁-C₆ alkyl optionally substituted with one or more substituents selected from halogen, NR₄R₅, CONR₄R₅, CN, OR₄ or COOR₄;
   (c) aryl,
   (d) heteroaryl,
   (e) cycloalkyl, or
   (f) heterocycloalkyl;
R₄ and R₅ are each independently selected from:
   (a) hydrogen or
   (b) C₁-C₆ alkyl optionally substituted with one or more substituents selected from OH, halogen, NH₂, CONH₂, CN, OCH₃, or COOH;
n is 0, 1, 2, 3 or 4;
   or a stereoisomer or a pharmaceutically acceptable salt thereof.

In another aspect, there are provided pharmaceutical compositions comprising a compound of Formula (I) or a stereoisomer or a pharmaceutically acceptable salt thereof.

In yet another aspect, there are provided methods for treating a bacterial infection in a subject, the methods comprising administering to the subject a pharmaceutically effective amount of a compound of Formula (I) or a stereoisomer or a pharmaceutically acceptable salt thereof.

In another aspect, there are provided methods for treating a bacterial infection in a subject, the methods comprising administering to the subject a pharmaceutically effective amount of a pharmaceutical composition comprising a compound of Formula (I) or a stereoisomer or a pharmaceutically acceptable salt thereof.

In yet another aspect, there are provided pharmaceutical compositions comprising: (a) a compound of Formula (I) or a stereoisomer or a pharmaceutically acceptable salt thereof, and (b) at least one antibacterial agent.

In another aspect, there are provided methods for treating a bacterial infection in a subject, the methods comprising administering to the subject a pharmaceutically effective amount of a pharmaceutical composition comprising: (a) a compound of Formula (I) or a stereoisomer or a pharmaceutically acceptable salt thereof, and (b) at least one antibacterial agent.

In another aspect, there are provided methods for treating a bacterial infection in a subject, the methods comprising administering to the subject a pharmaceutically effective amount of: (a) a compound of Formula (I) or a stereoisomer or a pharmaceutically acceptable salt thereof, and (b) at least one antibacterial agent.

In yet another aspect, there are provided methods for increasing antibacterial effectiveness of an antibacterial agent in a subject, the methods comprising co-administering said antibacterial agent with a compound of Formula (I) or a stereoisomer or a pharmaceutically acceptable salt thereof.

The details of one or more embodiments of the invention are set forth in the description below. Other features, objects and advantages of the invention will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, which would occur to one skilled in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety.

The term “C₁-C₆ alkyl” as used herein refers to branched or unbranched acyclic hydrocarbon radical with 1 to 6 carbon atoms. Typical non-limiting examples of “C₁-C₆ alkyl” include methyl, ethyl, n-propyl, iso-propyl, n-butyl, sec-butyl, iso-butyl, tert-butyl, n-pentyl, iso-pentyl, tert-pentyl, neopentyl, sec-pentyl, 3-pentyl, n-hexyl, 2-methylpentyl, 3-methylpentyl, 2,2-dimethylbutyl, 2,3-dimethylbutyl and the like. The “C₁-C₆ alkyl” may be unsubstituted, or substituted with one or more substituents. Typical, non-limiting examples of such substituents include halogen, alkoxy, CN, SH, COOH, COOC₁-C₆alkyl, CONH₂, OH, NH₂, NHCOCH₃, cycloalkyl, heterocycloalkyl, heteroaryl, aryl and the like.

The term “cycloalkyl” as used herein refers to three to seven member cyclic hydrocarbon radicals. The cycloalkyl group optionally incorporates one or more double or triple bonds, or a combination of double or triple bonds, but which is not aromatic. Typical, non-limiting examples of cycloalkyl groups include cyclopropyl, cyclobutyl, cyclopentyl, cyclohexyl, and cycloheptyl. The cycloalkyl may be unsubstituted, or substituted with one or more substituents. Typical, non-limiting examples of such substituents include C₁-C₆ alkyl, halogen, alkoxy, CN, SH, COOH, COOC₁-C₆alkyl, CONH₂, OH, NH₂, NHCOCH₃, heterocycloalkyl, heteroaryl, aryl, SO₂-alkyl, SO₂-aryl, OSO₂-alkyl, OSO₂-aryl and the like.

The term “aryl” as used herein refers to a monocyclic or polycyclic aromatic hydrocarbon. Typical, non-limiting examples of aryl groups include phenyl, naphthyl, anthracenyl, flourenyl, phenanthrenyl, indenyl and the like. The aryl group may be unsubstituted, or substituted with one or more substituents. Typical, non-limiting examples of such substituents include C₁-C₆ alkyl, halogen, alkoxy, CN, COOH, CONH₂, OH, NH₂, NHCOCH₃, heterocycloalkyl, heteroaryl, aryl, SO₂-alkyl, SO₂-aryl, OSO₂-alkyl, OSO₂-aryl and the like. The term “aryl” includes six to fourteen membered monocyclic or polycyclic aromatic hydrocarbon.

The term “heteroaryl” as used herein refers to a monocyclic or polycyclic aromatic hydrocarbon group wherein one or more carbon atoms have been replaced with heteroatoms selected from nitrogen, oxygen, and sulfur. If the heteroaryl group contains more than one heteroatom, the heteroatoms may be the same or different. Typical, non-limiting example of heteroaryl groups include pyridyl, pyrimidinyl, pyrazinyl, pyridazinyl, triazinyl, furanyl, pyrrolyl, thienyl, oxadiazolyl, thiadiazolyl, tetrazolyl, oxazolyl, thiazolyl, imidazolyl, pyrazolyl, triazonyl, isoxazolyl, oxadiazolyl, oxatriazolyl, isothiazolyl, thiatriazolyl, thiazinyl, oxazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo-pyridazinyl, purinyl, benzofuranyl, isobenzofuranyl, benzothienyl, benzothiophenyl, carbazolyl, benzimidazolyl, benzoxazolyl, benzoisoxazolyl, benzothiazolyl, benzotriazolyl, indolyl, isoindolyl, quinolinyl, isoquinolinyl, acridinyl, naphthothienyl, thianthrenyl, chromenyl, xanthenyl, phenoxathienyl, indolizinyl,indazolyl, phthalazinyl, naphthyridinyl, qinoxalinyl, quinazolinyl, cinnolinyl, pteridinyl, beta-carbolinyl, phenanthridinyl, phenanthrolinyl, phenazinyl, phenothiazinyl, phenoxazinyl and the like. The heteroaryl group may be unsubstituted, or substituted with one or more substituents. Typical, non-limiting examples of such substituents include C₁-C₆ alkyl, halogen, alkoxy, CN, COOH, CONH₂, OH, SH, SCH₃, NH₂, NHCOCH₃, heterocycloalkyl, heteroaryl, aryl, SO₂-alkyl, SO₂-aryl, OSO₂-alkyl, OSO₂-aryl and the like. The term “heteroaryl” includes five to fourteen membered monocyclic or polycyclic aromatic hydrocarbon group containing at least one heteroatom selected from nitrogen, oxygen, and sulfur.

The term “heterocycloalkyl” as used herein refers to three to seven member cycloalkyl group containing one or more heteroatoms selected from nitrogen, oxygen or sulfur. The heterocycloalkyl group optionally incorporates one or more double or triple bonds, or a combination of double bonds and triple bonds, but which is not aromatic. Typical, non-limiting example of heterocycloalkyl groups include aziridinyl, azetidinyl, pyrrolidinyl, 2-oxo-pyrrolidinyl, imidazolidin-2-one-yl, piperidinyl, oxazinyl, thiazinyl, piperazinyl, piperazin-2,3-dione-yl, morpholinyl, thiomorpholinyl, azepanyl, and the like. The heterocycloalkyl may be unsubstituted, or substituted with one or more substituents. Typical, non-limiting examples of such substituents include C₁-C₆ alkyl, halogen, alkoxy, CN, COOH, CONH₂, OH, NH₂, NHCOCH₃, heteroaryl, aryl, SO₂-alkyl, SO₂-aryl, OSO₂-aryl and the like. The term “heterocycloalkyl” includes three to seven membered cycloalkyl containing at least one heteroatom selected from nitrogen, oxygen, and sulfur.

The term “halogen” or halo as used herein refers to chlorine, bromine, fluorine or iodine.

The term “Bn” as used herein refers to benzyl group.

The term “Boc” as used herein refers to tert-butyloxycarbonyl group

The term “stereoisomers” as used herein refers to compounds that have identical chemical constitution, but differ with regard to the arrangement of their atoms or groups in space. The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, are embraced within the scope of the invention. In general, a reference to a compound is intended to cover its stereoisomers and mixture of various stereoisomers.

The term “optionally substituted” as used herein means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound.

The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the parent compound.

The term “pharmaceutically acceptable salt” as used herein refers to one or more salts of a given compound which possesses the desired pharmacological activity of the free compound and which are neither biologically nor otherwise undesirable. In general, the “pharmaceutically acceptable salts” refer to salts that are suitable for use in contact with the tissues of human and animals without undue toxicity, irrigation, allergic response and the like, and are commensurate with a reasonable benefit/risk ratio. Pharmaceutically acceptable salts are well known in the art. For example, S. M. Berge, et al. (J. Pharmaceutical Sciences, 66; 1-19, 1977), incorporated herein by reference in its entirety, describes various pharmaceutical acceptable salts in details.

In general, the compounds according to the invention contain basic (e.g. nitrogen atoms) as well as acid moieties. A person of skills in the art would appreciate that such compounds, therefore, can form acidic salts (formed with inorganic and/or organic acids), as well as basic salts (formed with inorganic and/or organic bases). Such salts can be prepared using procedures described in the art. For example, the basic moiety can be converted to its salt by treating a compound with a suitable amount of acid. Typical, non-limiting examples of such suitable acids include hydrochloric acid, trifluoroacetic acid, methanesulfonic acid or the like. Alternatively, the acid moiety may be converted into its salt by treating with a suitable base. Typical non-limiting examples of such bases include sodium carbonate, sodium bicarbonate, sodium ethylhexanoate, potassium carbonate, potassium bicarbonate, potassium ethyl hexanoate or the like. In case of compounds containing more than one functional group capable of being converted into salt, each such functional group may be converted to salt independently. For example, in case of compounds containing two basic nitrogen atoms, one of the basic nitrogen can form salt with one acid while the other basic nitrogen can form salt with another acid. Some compounds according to the invention contain both acidic as well as basic moieties, and thus can form inner salts or corresponding zwitterions. In general, all pharmaceutically acceptable salt forms of compound of Formula (I) according to invention including acid addition salts, base addition salts, zwitterions or the like are contemplated to be within the scope of the present invention and are generically referred to as pharmaceutically acceptable salts.

The term “infection” or “bacterial infection” as used herein includes presence of bacteria, in or on a subject, which, if its growth were inhibited, would result in a benefit to the subject. As such, the term “infection” in addition to referring to the presence of bacteria also refers to presence of other floras, which are not desirable. The term “infection” includes infection caused by bacteria.

The term “treat”, “treating” or “treatment” as used herein refers to administration of a medicament, including a pharmaceutical composition, or one or more pharmaceutically active ingredients, for prophylactic and/or therapeutic purposes. The term “prophylactic treatment” refers to treating a subject who is not yet infected, but who is susceptible to, or otherwise at a risk of infection (preventing the bacterial infection). The term “therapeutic treatment” refers to administering treatment to a subject already suffering from infection. The terms “treat”, “treating” or “treatment” as used herein also refer to administering compositions, or one or more of pharmaceutically active ingredients discussed herein, with or without additional pharmaceutically active or inert ingredients, in order to: (i) reduce or eliminate either a bacterial infection, or one or more symptoms of a bacterial infection, or (ii) retard progression of a bacterial infection, or one or more symptoms of a bacterial infection, or (iii) reduce severity of a bacterial infection, or one or more symptoms of a bacterial infection, or (iv) suppress clinical manifestation of a bacterial infection, or (v) suppress manifestation of adverse symptoms of a bacterial infection.

The terms “pharmaceutically effective amount” or “therapeutically effective amount” or “effective amount” as used herein refer to an amount, which has a therapeutic effect or is the amount required to produce a therapeutic effect in a subject. For example, a “therapeutically effective amount” or “pharmaceutically effective amount” or “effective amount” of an antibacterial agent or a pharmaceutical composition is the amount of the antibacterial agent or the pharmaceutical composition required to produce a desired therapeutic effect as may be judged by clinical trial results, model animal infection studies, and/or in vitro studies (e.g. in agar or broth media). Such effective amount depends on several factors, including but not limited to, the microorganism (e.g. bacteria) involved, characteristics of the subject (for example height, weight, sex, age and medical history), severity of infection and particular type of the antibacterial agent used. For prophylactic treatments, a prophylactically effective amount is that amount which would be effective in preventing the bacterial infection.

The term “administration” or “administering” refers to and includes delivery of a composition, or one or more pharmaceutically active ingredients to a subject, including for example, by any appropriate method, which serves to deliver the composition or its active ingredients or other pharmaceutically active ingredients to the site of infection. The method of administration may vary depending on various factors, such as for example, the components of the pharmaceutical composition or type/nature of the pharmaceutically active or inert ingredients, site of the potential or actual infection, the microorganism involved, severity of the infection, age and physical condition of the subject and a like. Some non-limiting examples of ways to administer a composition or a pharmaceutically active ingredient to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop and mouthwash. In case of a pharmaceutical composition comprising more than one ingredients (active or inert), one of the ways of administering such composition is by admixing the ingredients (e.g. in the form of a suitable unit dosage form such as tablet, capsule, solution, powder or a like) and then administering the dosage form. Alternatively, the ingredients may also be administered separately (simultaneously or one after the other) as long as these ingredients reach beneficial therapeutic levels such that the composition as a whole provides a synergistic and/or desired effect.

The term “growth” as used herein refers to a growth of one or more microorganisms and includes reproduction or population expansion of the microorganism (e.g. bacteria). The term “growth” also includes maintenance of on-going metabolic processes of the microorganism, including the processes that keep the microorganism alive.

The term, “effectiveness” as used herein refers to ability of a treatment, or a composition, or one or more pharmaceutically active ingredients to produce a desired biological effect in a subject. For example, the term “antibacterial effectiveness” of a composition or of an antibacterial agent refers to the ability of the composition or the antibacterial agent to prevent or treat bacterial infection in a subject.

The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

The term “beta-lactamase” or “beta-lactamase enzyme” as used herein refers to any enzyme or protein or any other substance that breaks down a beta-lactam ring. The term “beta-lactamase” includes enzymes that are produced by bacteria and have the ability to hydrolyze the beta-lactam ring in a beta-lactam compound, either partially or completely.

The term “beta-lactamase inhibitor” as used herein refers to a compound capable of inhibiting activity of one or more beta-lactamase enzymes, either partially or completely.

The term “pharmaceutically inert ingredient” or “carrier” or “excipient” refers to and includes compounds or materials used to facilitate administration of a compound, for example, to increase the solubility of the compound. Typical, non-limiting examples of solid carriers include starch, lactose, dicalcium phosphate, sucrose, and kaolin. Typical, non-limiting examples of liquid carriers include sterile water, saline, buffers, non-ionic surfactants, and edible oils. In addition, various adjuvants commonly used in the art may also be included. These and other such compounds are described in literature, e.g., in the Merck Index (Merck & Company, Rahway, N.J.). Considerations for inclusion of various components in pharmaceutical compositions are described, e.g., in Gilman et al. (Goodman and Gilman's: The Pharmacological Basis of Therapeutics, 8th Ed., Pergamon Press., 1990), which is incorporated herein by reference in its entirety.

The term “subject” as used herein refers to vertebrate or invertebrate, including a mammal. The term “subject” includes human, animal, a bird, a fish, or an amphibian. Typical, non-limiting examples of a “subject” include humans, cats, dogs, horses, sheep, bovine cows, pigs, lambs, rats, mice and guinea pigs.

In one aspect, there are provided compounds of Formula (I):

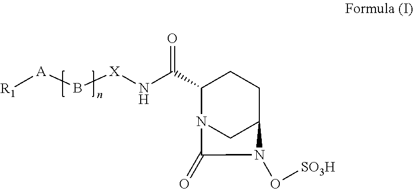

wherein:
A is a four to six membered nitrogen containing ring optionally substituted with one or more substituents selected from C₁-C₆ alkyl, oxo, NR₂R₃, aryl, heteroaryl, cycloalkyl or heterocycloalkyl;
B is C═O or CH—Z;
X is O or CONH;
Z is H, C₁-C₆ alkyl, OR₂ or NR₂₃;
R₁ is selected from:
   (a) hydrogen,
   (b) C₁-C₆ alkyl optionally substituted with one or more substituents independently selected from OR₂, NR₂R₃, SR₂, halogen, CN, COOR₂, CONR₂R₃, SR₂, CH₂OR₂, CH₂NR₂R₃, OCOR₂, aryl, heteroaryl, cycloalkyl or heterocycloalkyl,
   (c) CN,
   (d) CONR₂R₃,
   (e) COOR₂,
   (f) NR₂R₃,
   (g) NHCOOR₂,
   (h) aryl,
   (i) heteroaryl,
   (j) cycloalkyl, or
   (k) heterocycloalkyl;
R₂ and R₃ are each independently selected from:
   (a) hydrogen,
   (b) C₁-C₆ alkyl optionally substituted with one or more substituents selected from halogen, NR₄R₅, CONR₄R₅, CN, OR₄ or COOR₄;
   (c) aryl,
   (d) heteroaryl,
   (e) cycloalkyl, or
   (f) heterocycloalkyl;
R₄ and R₅ are each independently selected from:
   (a) hydrogen or
   (b) C₁-C₆ alkyl optionally substituted with one or more substituents selected from OH, halogen, NH₂, CONH₂, CN, OCH₃, or COOH;
n is 0, 1, 2, 3 or 4;
   or a stereoisomer or a pharmaceutically acceptable salt thereof.

In some embodiments, there are provided compounds of Formula (I) wherein X is CONH₂.

In some other embodiments, there are provided compounds of Formula (I) wherein X is O.

In some embodiments, there are provided compounds of Formula (I) wherein A is attached to B through nitrogen atom.

In some embodiments, there are provided compounds of Formula (I) wherein A is five membered nitrogen containing heteroaryl ring.

In some other embodiments, there are provided compounds of Formula (I) wherein A is five membered nitrogen containing heteroaryl ring and A is attached to B through nitrogen atom

In some embodiments, there are provided compounds of Formula (I) wherein X is CONH₂, A is five membered nitrogen containing heteroaryl ring and A is attached to B through nitrogen atom.

In some other embodiments, there are provided compounds of Formula (I) wherein X is O, A is five membered nitrogen containing heteroaryl ring and A is attached to B through nitrogen atom.

In other embodiments, there are provided compounds of Formula (I) wherein A is tetrazole, pyrrole, imidazole or triazole.

In some other embodiments, there are provided compounds of Formula (I) wherein X is CONH₂, A is tetrazole, pyrrole, imidazole or triazole, and A is attached to B through nitrogen atom.

In some embodiments, there are provided compounds of Formula (I) wherein X is O, A is tetrazole, pyrrole, imidazole or triazole and A is attached to B through nitrogen atom.

Typical, non-limiting examples of compounds according to the invention include:

(2S,5R)-7-oxo-6-(sulfooxy)-N′-(H-tetrazol-5-ylcarbonyl)-1,6-diazabicyclo[3.2.1]octane-2-carbohydrazide;

(2S,5R)-N′-(1H-imidazol-1-yl-acetyl)-7-oxo-6-(sulfooxy)-1,6-diazabicyclo[3.2.1]octane-2-carbohydrazide;

(2S,5R)-7-oxo-6-(sulfooxy)-N′-(1H-tetrazol-1-ylacetyl)-1,6-diazabicyclo[3.2.1]octane-2-carbohydrazide;

(2S,5R)-7-oxo-6-(sulfooxy)-N′-(2H-tetrazol-2-ylacetyl)-1,6-diazabicyclo[3.2.1]octane-2-carbohydrazide;

(2S,5R)-N′-{[5-(ethoxycarbonyl)-1H-tetrazol-1-yl]acetyl}-7-oxo-6-(sulfooxy)-1,6-diazabicyclo[3.2.1]octane-2-carbohydrazide;

(2S,5R)-7-oxo-6-(sulfooxy)-N′-(2H-1,2,3-triazol-2-ylacetyl)-1,6-diazabicyclo[3.2.1]octane-2-carbohydrazide;

(2S,5R)-7-oxo-6-(sulfooxy)-N′-(1H-1,2,3-triazol-1-ylacetyl)-1,6-diazabicyclo[3.2.1]octane-2-carbohydrazide;

(2S,5R)-7-oxo-6-(sulfooxy)-N′-(1H-1,2,4-triazol-1-ylacetyl)-1,6-diazabicyclo[3.2.1]octane-2-carbohydrazide;

(2S,5R)-N′-{[5-methyl-1H-tetrazol-1-yl]acetyl}-7-oxo-6-(sulfooxy)-1,6-diazabicyclo[3.2.1]octane-2-carbohydrazide;

(2S,5R)-N′-{[4-methyl-1H-tetrazol-1-yl]acetyl}-7-oxo-6-(sulfooxy)-1,6-diazabicyclo[3.2.1]octane-2-carbohydrazide;

(2S,5R)-7-oxo-N′-(1H-pyrazol-1-ylacetyl)-6-(sulfooxy)-1,6-diazabicyclo[3.2.1]octane-2-carbohydrazide;

(2S,5R)-N′-[(5-amino-1H-tetrazol-1-yl)acetyl]-7-oxo-6-(sulfooxy)-1,6-diazabicyclo[3.2.1]octane-2-carbohydrazide;

(2S,5R)-N′-{[5-(aminomethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(aminomethyl)-2H-tetrazol-2-yl]acetyl}-7-oxo-6-(sulfooxy)-1,6-diazabicyclo[3.2.1]octane-2-carbohydrazide;

(2S,5R)-N′-[(3-amino-1H-1,2,4-triazol-1-yl)acetyl]-7-oxo-6-(sulfooxy)-1,6-diazabicyclo[3.2.1]octane-2-carbohydrazide;

(2S,5R)-7-oxo-6-(sulfooxy)-N′-(H-tetrazol-5-ylacetyl)-1,6-diazabicyclo[3.2.1]octane-2-carbohydrazide;

(2S,5R)-N′-[(5-carbamoyl-1H-tetrazol-1-yl)acetyl]-7-oxo-6-(sulfooxy)-1,6-diazabicyclo[3.2.1]octane-2-carbohydrazide;

(2S,5R)-N′-[(5-carbamoyl-2H-tetrazol-2-yl)acetyl]-7-oxo-6-(sulfooxy)-1,6-diazabicyclo[3.2.1]octane-2-carbohydrazide;

(2S,5R)-N′-[(5-cyano-1H-tetrazol-1-yl)acetyl]-7-oxo-6-(sulfooxy)-1,6-diazabicyclo[3.2.1]octane-2-carbohydrazide;

(2S,5R)-N′-[(5-cyano-2H-tetrazol-2-yl)acetyl]-7-oxo-6-(sulfooxy)-1,6-diazabicyclo[3.2.1]octane-2-carbohydrazide;

(2S,5R)-7-oxo-6-(sulfooxy)-N′-[(2S)-2-(1H-tetrazol-1-yl)propanoyl]-1,6-diazabicyclo[3.2.1]octane-2-carbohydrazide;

(2S,5R)-7-oxo-6-(sulfooxy)-N′-[(2R)-2-(1H-tetrazol-1-yl)propanoyl]-1,6-diazabicyclo[3.2.1]octane-2-carbohydrazide;

(2S,5R)-N′-{[5-(2-ethoxy-2-oxoethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(2-ethoxy-2-oxoethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(cyanomethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(cyanomethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(2-hydroxyethyl)-2H-tetrazol-1-yl]acetyl}-7-oxo-6-(sulfooxy)-1,6-diazabicyclo[3.2.1]octane-2-carbohydrazide;

(2S,5R)-N′-{[2-(2-aminoethyl)-2H-tetrazol-5-yl]acetyl}-7-oxo-6-(sulfooxy)-1,6-diazabicyclo[3.2.1]octane-2-carbohydrazide;

(2S,5R)-N′-{[1-(2-aminoethyl)-1H-tetrazol-5-yl]acetyl}-7-oxo-6-(sulfooxy)-1,6-diazabicyclo[3.2.1]octane-2-carbohydrazide;

(2S,5R)-N′-{[4-(aminomethyl)-2H-1,2,3-triazol-2-yl]acetyl}-7-oxo-6-(sulfooxy)-1,6-diazabicyclo[3.2.1]octane-2-carbohydrazide;

(2S,5R)-N′-{[4-(aminomethyl)-1H-1,2,3-triazol-1-yl]acetyl}-7-oxo-6-(sulfooxy)-1,6-diazabicyclo[3.2.1]octane-2-carbohydrazide;

(2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide;

(2S,5R)-N′-{[4-(1-methoxy-1-oxomethyl)-1H-1,2,3-triazol-1-yl]acetyl}-7-oxo-6-(sulfooxy)-1,6-diazabicyclo[3.2.1]octane-2-carbohydrazide;

(2S,5R)-N′-{[4-carbamoyl-1H-1,2,3-triazol-1-yl]acetyl}-7-oxo-6-(sulfooxy)-1,6-diazabicyclo[3.2.1]octane-2-carbohydrazide;

(2S,5R)-N′-[(4-cyano-1H-1,2,3-triazol-1-yl)acetyl]-7-oxo-6-(sulfooxy)-1,6-diazabicyclo[3.2.1]octane-2-carbohydrazide;

(2S,5R)-N′-{[5-(2-aminoethoxy)-1H-tetrazol-1-yl]acetyl}-7-oxo-6-(sulfooxy)-1,6-diazabicyclo[3.2.1]octane-2-carbohydrazide;

(2S,5R)-N′-{[5-(2-amino-1-oxo-ethoxy)-1H-tetrazol-1-yl]acetyl}-7-oxo-6-(sulfooxy)-1,6-diazabicyclo[3.2.1]octane-2-carbohydrazide;

(2S,5R)-N′-[(2-methyl-1H-imidazol-1-yl)acetyl]-7-oxo-6-(sulfooxy)-1,6-diazabicyclo[3.2.1]octane-2-carbohydrazide;

(2S,5R)-N′-[(2,4-dimethyl-1H-imidazol-1-yl)acetyl]-7-oxo-6-(sulfooxy)-1,6-diazabicyclo[3.2.1]octane-2-carbohydrazide;

(2S,5R)-N′-[(2,5-dimethyl-1H-imidazol-1-yl)acetyl]-7-oxo-6-(sulfooxy)-1,6-diazabicyclo[3.2.1]octane-2-carbohydrazide;

(2S,5R)-N′-{[2-(1-methoxy-1-oxomethyl)-1H-imidazol-1-yl]acetyl}-7-oxo-6-(sulfooxy)-1,6-diazabicyclo[3.2.1]octane-2-carbohydrazide;

(2S,5R)-N′-{[2-(1-methoxy-1-oxomethyl)-5-methyl-1H-imidazol-1-yl]acetyl}-7-oxo-6-(sulfooxy)-1,6-diazabicyclo[3.2.1]octane-2-carbohydrazide;
(2S,5R)-N′-{[2-phenyl-5-methyl-1H-imidazol-1-yl]acetyl}-7-oxo-6-(sulfooxy)-1,6-diazabicyclo[3.2.1]octane-2-carbohydrazide;

(2S,5R)-N′-(1H-benzimidazol-1-ylacetyl)-7-oxo-6-(sulfooxy)-1,6-diazabicyclo[3.2.1]octane-2-carbohydrazide;

(2S,5R)-N′-{[5-(2-aminoethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-aminoethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(3-aminopropyl)-2H-tetrazol-2-yl]acetyl}-7-oxo-6-(sulfooxy)-1,6-diazabicyclo[3.2.1]octane-2-carbohydrazide;

(2S,5R)-N′-{[5-(3-aminopropyl)-1H-tetrazol-1-yl]acetyl}-7-oxo-6-(sulfooxy)-1,6-diazabicyclo[3.2.1]octane-2-carbohydrazide;

(2S,5R)-N′-{[5-(4-aminobutyl)-2H-tetrazol-2-yl]acetyl}-7-oxo-6-(sulfooxy)-1,6-diazabicyclo[3.2.1]octane-2-carbohydrazide;

(2S,5R)-N′-{[5-(4-aminobutyl)-1H-tetrazol-1-yl]acetyl}-7-oxo-6-(sulfooxy)-1,6-diazabicyclo[3.2.1]octane-2-carbohydrazide;

(2S,5R)-N′-[(2-methyl-1H-imidazol-1-yl)acetyl]-7-oxo-6-(sulfooxy)-1,6-diazabicyclo[3.2.1]octane-2-carbohydrazide;

(2S,5R)-N′-[(2S)-2-(1H-imidazol-1-yl)propanoyl]-7-oxo-6-(sulfooxy)-1,6-diazabicyclo[3.2.1]octane-2-carbohydrazide;

(2S,5R)-N′-{[2-({[(2S)-pyrrolidin-2-ylcarbonyl]oxy}methyl)-1H-imidazol-1-yl]acetyl}-7-oxo-6-(sulfooxy)-1,6-diazabicyclo[3.2.1]octane-2-carbohydrazide;

(2S,5R)-N′-{[2-({[(2S)-2-aminopropanoyl]oxy}methyl)-1H-imidazol-1-yl]acetyl}-7-oxo-6-(sulfooxy)-1,6-diazabicyclo[3.2.1]octane-2-carbohydrazide;

(2S,5R)-N′-{[5-(2-oxo-2-oxyethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(2-oxo-2-oxyethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;

(2S,5R)-N′-{[5-(carbamoyl)-2H-tetrazol-2-yl]acetyl}-7-oxo-6-(sulfooxy)-1,6-diazabicyclo[3.2.1]octane-2-carbohydrazide;

(2S,5R)-N′-{[5-(carbamoyl)-1H-tetrazol-1-yl]acetyl}-7-oxo-6-(sulfooxy)-1,6-diazabicyclo[3.2.1]octane-2-carbohydrazide;

(2S,5R)-7-oxo-N′-(1H-pyrrol-1-ylacetyl)-6-(sulfooxy)-1,6-diazabicyclo[3.2.1]octane-2-carbohydrazide;

(2S,5R)-N′-{[5-(2-hydroxyethyl)-2H-tetrazol-2-yl]acetyl}-7-oxo-6-(sulfooxy)-1,6-diazabicyclo[3.2.1]octane-2-carbohydrazide;

(2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;

(2S,5R)-N′-{[2-(2-hydroxyethyl)-1H-imidazol-1-yl]acetyl}-7-oxo-6-(sulfooxy)-1,6-diazabicyclo[3.2.1]octane-2-carbohydrazide;

(2S, 5R)-N′-{[4-amino-1H-imidazol-1-yl]-acetyl}-7-oxo-6-(sulfooxy)-1,6-diazabicyclo[3.2.1]octane-2-carbohydrazide;

(2S, 5R)-N′-{[4-amino-2-oxopyrimidin-1(2H)-yl]acetyl}-7-oxo-6-(sulfooxy)-1,6-diazabicyclo[3.2.1]octane-2-carbohydrazide;

(2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide;

Mixture of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide and (2S,5R)-N′-{[5-(hydroxymethyl)-1H-1,2,3-triazol-1-yl]acetyl}-7-oxo-6-(sulfooxy)-1,6-diazabicyclo[3.2.1]octane-2-carbohydrazide;

(2S,5R)-N′-{[2-(hydroxymethyl)-1H-imidazol-1-yl]acetyl}-7-oxo-6-(sulfooxy)-1,6-diazabicyclo[3.2.1]octane-2-carbohydrazide;

(2S,5R)-N′-[2-(ethoxycarbonyl)-1H-imidazol-1-ylacetyl]-7-oxo-6-(sulfooxy)-1,6-diazabicyclo[3.2.1]octane-2-carbohydrazide;

(2S,5R)-N′-[2-(carbamoyl)-1H-imidazol-1-ylacetyl]-7-oxo-6-(sulfooxy)-1,6-diazabicyclo[3.2.1]octane-2-carbohydrazide;

(2S,5R)-N′-[2-(methoxycarbonyl)-1H-imidazol-1-ylacetyl]-7-oxo-6-(sulfooxy)-1,6-diazabicyclo[3.2.1]octane-2-carbohydrazide;

(2S,5R)-N-{2-[5-(aminomethyl)-2H-tetrazol-2-yl]ethoxy}-7-oxo-6-(sulfooxy)-1,6-diazabicyclo[3.2.1]octane-2-carboxamide;

(2S,5R)-N-(2-(2H-1,2,3-triazol-2-yl)ethoxy)-6-(sulfooxy)-7-oxo-1,6-diaza-bicyclo[3.2.1]octane-2-carboxamide;

(2S,5R)-N-(2-(1H-1,2,3-triazol-1-yl)ethoxy)-6-(sulfooxy)-7-oxo-1,6-diaza-bicyclo[3.2.1]octane-2-carboxamide;

(2S,5R)-7-oxo-6-(sulfooxy)-N-[2-(1H-1,2,4-triazol-1-yl)ethoxy]-1,6-diazabicyclo[3.2.1]octane-2-carboxamide;

(2S,5R)-N-{2-[5-(ethoxycarbonyl)-1H-tetrazol-1-yl]ethoxy}-6-sulfooxy-7-oxo-1,6-diazabicyclo[3.2.1]octane-2-carboxamide;

(2S,5R)-7-oxo-6-(sulfooxy)-N-[2-(2H-tetrazol-2-yl)ethoxy]-1,6-diazabicyclo[3.2.1]octane-2-carboxamide;

(2S,5R)-7-oxo-6-(sulfooxy)-N-[2-(1H-tetrazol-1-yl)ethoxy]-1,6-diazabicyclo[3.2.1]octane-2-carboxamide;

(2S,5R)-N-[2-(5-methyl-1H-tetrazol-1-yl)ethoxy]-7-oxo-6-(sulfooxy)-1,6-diazabicyclo[3.2.1]octane-2-carboxamide;

(2S,5R)-N-[2-(5-methyl-2H-tetrazol-2-yl)ethoxy]-7-oxo-6-(sulfooxy)-1,6-diazabicyclo[3.2.1]octane-2-carboxamide;

(2S,5R)-N-[2-(3-amino-1H-1,2,4-triazol-1-yl)ethoxy]-7-oxo-6-(sulfooxy)-1,6-diazabicyclo[3.2.1]octane-2-carboxamide;

(2S,5R)-N-[2-(5-amino-1H-tetrazol-1-yl)ethoxy]-7-oxo-6-(sulfooxy)-1,6-diazabicyclo[3.2.1]octane-2-carboxamide;

(2S,5R)-N-[2-(5-amino-2H-tetrazol-2-yl)ethoxy]-7-oxo-6-(sulfooxy)-1,6-diazabicyclo[3.2.1]octane-2-carboxamide;

(2S,5R)-N-[2-(5-carbamoyl-1H-tetrazol-1-yl)ethoxy]-7-oxo-6-(sulfooxy)-1,6-diazabicyclo[3.2.1]octane-2-carboxamide;

(2S,5R)-N-{2-[5-(2-ethoxy-2-oxoethyl)-2H-tetrazol-2-yl]ethoxy}-6-sulfooxy-7-oxo-1,6-diazabicyclo[3.2.1]octane-2-carboxamide;

(2S,5R)-N-{2-[5-(2-ethoxy-2-oxoethyl)-1H-tetrazol-1-yl]ethoxy}-6-(sulfooxy)-7-oxo-1,6-diazabicyclo[3.2.1]octane-2-carboxamide;

(2S,5R)-N-{2-[5-(2-amino-2-oxoethyl)-2H-tetrazol-2-yl]ethoxy}-7-oxo-6-(sulfooxy)-1,6-diazabicyclo[3.2.1]octane-2-carboxamide;

(2S,5R)-N-{2-[5-(2-amino-2-oxoethyl)-1H-tetrazol-1-yl]ethoxy}-7-oxo-6-(sulfooxy)-1,6-diazabicyclo[3.2.1]octane-2-carboxamide;

(2S,5R)-N-{2-[5-(amino methoxy)-1H-tetrazol-1-yl]ethoxy}-7-oxo-6-(sulfooxy)-1,6-diazabicyclo[3.2.1]octane-2-carboxamide;

(2S,5R)-N-{2-[5-(2-aminoethoxy)-1H-tetrazol-1-yl]ethoxy}-7-oxo-6-(sulfooxy)-1,6-diazabicyclo[3.2.1]octane-2-carboxamide;

(2S,5R)-N-{2-[5-(3-aminopropoxy)-1H-tetrazol-1-yl]ethoxy}-6-(sulfooxy)-7-oxo-1,6-diazabicyclo[3.2.1]octane-2-carboxamide;

(2S,5R)-N-{2-[5-(2-amino-1-oxo-ethoxy)-1H-tetrazol-1-yl]ethoxy}-7-oxo-6-(sulfooxy)-1,6-diazabicyclo[3.2.1]octane-2-carboxamide;

(2S,5R)-N-{2-[5-(2-aminoethyl)-2H-tetrazol-2-yl]ethoxy}-7-oxo-6-(sulfooxy)-1,6-diazabicyclo[3.2.1]octane-2-carboxamide;

(2S,5R)-N-{2-[4-(hydroxymethyl)-2H-1,2,3-triazol-2-yl]ethoxy}-7-oxo-6-(sulfooxy)-1,6-diazabicyclo[3.2.1]octane-2-carboxamide;

(2S,5R)-N-{2-[5-(2-hydroxyethyl)-2H-tetrazol-2-yl]ethoxy}-7-oxo-6-(sulfooxy)-1,6-diazabicyclo[3.2.1]octane-2-carboxamide;

(2S,5R)-N-{2-[5-(2-hydroxyethyl)-1H-tetrazol-1-yl]ethoxy}-7-oxo-6-(sulfooxy)-1,6-diazabicyclo[3.2.1]octane-2-carboxamide;

(2S,5R)-N-{2-[5-(2-oxo-2-ethoxyethyl)-2H-tetrazol-2-yl]ethoxy}-7-oxo-6-(sulfooxy)-1,6-diazabicyclo[3.2.1]octane-2-carboxamide;

or a stereoisomer or a pharmaceutically acceptable salt thereof.

In some other embodiments, typical, non-limiting examples of compounds according to the invention include:

Di sodium salt of (2S,5R)-7-oxo-6-(sulfooxy)-N′-(H-tetrazol-5-ylcarbonyl)-1,6-diazabicyclo[3.2.1]octane-2-carbohydrazide;

Sodium salt of (2S,5R)-N′-(1H-imidazol-1-yl-acetyl)-7-oxo-6-(sulfooxy)-1,6-diazabicyclo[3.2.1]octane-2-carbohydrazide;

Sodium salt of (2S,5R)-7-oxo-6-(sulfooxy)-N′-(1H-tetrazol-1-ylacetyl)-1,6-diazabicyclo[3.2.1]octane-2-carbohydrazide;

Sodium salt of (2S,5R)-7-oxo-6-(sulfooxy)-N′-(2H-tetrazol-2-ylacetyl)-1,6-diazabicyclo[3.2.1]octane-2-carbohydrazide;

Sodium salt of (2S,5R)-N′-{[5-(ethoxycarbonyl)-1H-tetrazol-1-yl]acetyl}-7-oxo-6-(sulfooxy)-1,6-diazabicyclo[3.2.1]octane-2-carbohydrazide;

Sodium salt of (2S,5R)-7-oxo-6-(sulfooxy)-N′-(2H-1,2,3-triazol-2-ylacetyl)-1,6-diazabicyclo[3.2.1]octane-2-carbohydrazide;

Sodium salt of (2S,5R)-7-oxo-6-(sulfooxy)-N′-(1H-1,2,3-triazol-1-ylacetyl)-1,6-diazabicyclo[3.2.1]octane-2-carbohydrazide;

Sodium salt of (2S,5R)-7-oxo-6-(sulfooxy)-N′-(1H-1,2,4-triazol-1-ylacetyl)-1,6-diazabicyclo[3.2.1]octane-2-carbohydrazide;

Sodium salt of (2S,5R)-N′-{[5-methyl-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[4-methyl-1H-tetrazol-1-yl]acetyl}-7-oxo-6-(sulfooxy)-1,6-diazabicyclo[3.2.1]octane-2-carbohydrazide;

Sodium salt of (2S,5R)-7-oxo-N′-(1H-pyrazol-1-ylacetyl)-6-(sulfooxy)-1,6-diazabicyclo[3.2.1]octane-2-carbohydrazide;

Sodium salt of (2S,5R)-N′-[(5-amino -1H-tetrazol-1-yl)acetyl]-7-oxo-6-(sulfooxy)-1,6-diazabicyclo[3.2.1]octane-2-carbohydrazide;

Sodium salt of (2S,5R)-N′-{[5-(aminomethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(aminomethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-[(3-amino-1H-1,2,4-triazol-1-yl)acetyl]-7-oxo-6-(sulfooxy)-1,6-diazabicyclo[3.2.1]octane-2-carbohydrazide;

Disodium salt of (2S,5R)-7-oxo-6-(sulfooxy)-N′-(H-tetrazol-5-ylacetyl)-1,6-diazabicyclo[3.2.1]octane-2-carbohydrazide;

Sodium salt of (2S,5R)-N′-[(5-carbamoyl-1H-tetrazol-1-yl)acetyl]-7-oxo-6-(sulfooxy)-1,6-diazabicyclo[3.2.1]octane-2-carbohydrazide;

Sodium salt of (2S,5R)-N′-[(5-carbamoyl-2H-tetrazol-2-yl)acetyl]-7-oxo-6-(sulfooxy)-1,6-diazabicyclo[3.2.1]octane-2-carbohydrazide;

Sodium salt of (2S,5R)-N′-[(5-cyano-1H-tetrazol-1-yl)acetyl]-7-oxo-6-(sulfooxy)-1,6-diazabicyclo[3.2.1]octane-2-carbohydrazide;

Sodium salt of (2S,5R)-N′-[(5-cyano-2H-tetrazol-2-yl)acetyl]-7-oxo-6-(sulfooxy)-1,6-diazabicyclo[3.2.1]octane-2-carbohydrazide;

Sodium salt of (2S,5R)-7-oxo-6-(sulfooxy)-N′-[(2S)-2-(1H-tetrazol-1-yl)propanoyl]-1,6-diazabicyclo[3.2.1]octane-2-carbohydrazide;

Sodium salt of (2S,5R)-7-oxo-6-(sulfooxy)-N′-[(2R)-2-(1H-tetrazol-1-yl)propanoyl]-1,6-diazabicyclo[3.2.1]octane-2-carbohydrazide;

Sodium salt of (2S,5R)-N′-{[5-(2-ethoxy-2-oxoethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(2-ethoxy-2-oxoethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(cyanomethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(cyanomethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(2-hydroxyethyl)-2H-tetrazol-1-yl]acetyl}-7-oxo-6-(sulfooxy)-1,6-diazabicyclo[3.2.1]octane-2-carbohydrazide;

Sodium salt of (2S,5R)-N′-{[2-(2-aminoethyl)-2H-tetrazol-5-yl]acetyl}-7-oxo-6-(sulfooxy)-1,6-diazabicyclo[3.2.1]octane-2-carbohydrazide;

Sodium salt of (2S,5R)-N′-{[1-(2-aminoethyl)-1H-tetrazol-5-yl]acetyl}-7-oxo-6-(sulfooxy)-1,6-diazabicyclo[3.2.1]octane-2-carbohydrazide;

Sodium salt of (2S,5R)-N′-{[4-(aminomethyl)-2H-1,2,3-triazol-2-yl]acetyl}-7-oxo-6-(sulfooxy)-1,6-diazabicyclo[3.2.1]octane-2-carbohydrazide;

Sodium salt of (2S,5R)-N′-{[4-(aminomethyl)-1H-1,2,3-triazol-1-yl]acetyl}-7-oxo-6-(sulfooxy)-1,6-diazabicyclo[3.2.1]octane-2-carbohydrazide;

Sodium salt of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide;

Sodium salt of (2S,5R)-N′-{[4-(1-methoxy-1-oxomethyl)-1H-1,2,3-triazol-1-yl]acetyl}-7-oxo-6-(sulfooxy)-1,6-diazabicyclo[3.2.1]octane-2-carbohydrazide;

Sodium salt of (2S,5R)-N′-{[4-carbamoyl-1H-1,2,3-triazol-1-yl]acetyl}-7-oxo-6-(sulfooxy)-1,6-diazabicyclo[3.2.1]octane-2-carbohydrazide;

Sodium salt of (2S,5R)-N′-[(4-cyano-1H-1,2,3-triazol-1-yl)acetyl]-7-oxo-6-(sulfooxy)-1,6-diazabicyclo[3.2.1]octane-2-carbohydrazide;

Sodium salt of (2S,5R)-N′-{[5-(2-aminoethoxy)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(2-amino-1-oxo-ethoxy)-1H-tetrazol-1-yl]acetyl}-7-oxo-6-(sulfooxy)-1,6-diazabicyclo[3.2.1]octane-2-carbohydrazide;

Sodium salt of (2S,5R)-N′-[(2-methyl-1H-imidazol-1-yl)acetyl]-7-oxo-6-(sulfooxy)-1,6-diazabicyclo[3.2.1]octane-2-carbohydrazide;

Sodium salt of (2S,5R)-N′-[(2,4-dimethyl-1H-imidazol-1-yl)acetyl]-7-oxo-6-(sulfooxy)-1,6-diazabicyclo[3.2.1]octane-2-carbohydrazide;

Sodium salt of (2S,5R)-N′-[(2,5-dimethyl-1H-imidazol-1-yl)acetyl]-7-oxo-6-(sulfooxy)-1,6-diazabicyclo[3.2.1]octane-2-carbohydrazide;

Sodium salt of (2S,5R)-N′-1[2-(1-methoxy-1-oxomethyl)-1H-imidazol-1-yl]acetyl}-7-oxo-6-(sulfooxy)-1,6-diazabicyclo[3.2.1]octane-2-carbohydrazide;

Sodium salt of (2S,5R)-N′-{[2-(1-methoxy-1-oxomethyl)-5-methyl-1H-imidazol-1-yl]acetyl}-7-oxo-6-(sulfooxy)-1,6-diazabicyclo[3.2.1]octane-2-carbohydrazide;

Sodium salt of (2S,5R)-N′-{[2-phenyl-5-methyl-1H-imidazol-1-yl]acetyl}-7-oxo-6-(sulfooxy)-1,6-diazabicyclo[3.2.1]octane-2-carbohydrazide;

Sodium salt of (2S,5R)-N-(1H-benzimidazol-1-ylacetyl)-7-oxo-6-(sulfooxy)-1,6-diazabicyclo[3.2.1]octane-2-carbohydrazide;

Sodium salt of (2S,5R)-N-{[5-(2-aminoethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-aminoethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(3-aminopropyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(3-aminopropyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(4-aminobutyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(4-aminobutyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-[(2-methyl-1H-imidazol-1-yl)acetyl]-7-oxo-6-(sulfooxy)-1,6-diazabicyclo[3.2.1]octane-2-carbohydrazide;

Sodium salt of (2S,5R)-N′-[(2S)-2-(1H-imidazol-1-yl)propanoyl]-7-oxo-6-(sulfooxy)-1,6-diazabicyclo[3.2.1] octane-2-carbohydrazide;

Sodium salt of (2S,5R)-N′-{[2-(1[(25)-pyrrolidin-2-ylcarbonyl]oxy}methyl)-1H-imidazol-1-yl]acetyl}-7-oxo-6-(sulfooxy)-1,6-diazabicyclo[3.2.1]octane-2-carbohydrazide;

Sodium salt of (2S,5R)-N′-{[2-({[(2S)-2-aminopropanoyl]oxy}methyl)-1H-imidazol-1-yl]acetyl}-7-oxo-6-(sulfooxy)-1,6-diazabicyclo[3.2.1]octane-2-carbohydrazide;

Sodium salt of (2S,5R)-N′-{[5-(2-oxo-2-oxyethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(2-oxo-2-oxyethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[5-(carbamoyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(carbamoyl)-1H-tetrazol-1-yl]acetyl}-7-oxo-6-(sulfooxy)-1,6-diazabicyclo[3.2.1]octane-2-carbohydrazide;

Sodium salt of (2S,5R)-7-oxo-N-(1H-pyrrol-1-ylacetyl)-6-(sulfooxy)-1,6-diazabicyclo[3.2.1]octane-2-carbohydrazide;

Sodium salt of (2S,5R)-N′-{[5-(2-hydroxyethyl)-2H-tetrazol-2-yl]acetyl}-7-oxo-6-(sulfooxy)-1,6-diazabicyclo[3.2.1]octane-2-carbohydrazide;

Sodium salt of (2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;

Sodium salt of (2S,5R)-N′-{[2-(2-hydroxyethyl)-1H-imidazol-1-yl]acetyl}-7-oxo-6-(sulfooxy)-1,6-diazabicyclo[3.2.1]octane-2-carbohydrazide;

Sodium and Trifluoroacetic acid salt of (2S, 5R)-N-{[4-amino-1H-imidazol-1-yl]-acetyl}-7-oxo-6-(sulfooxy)-1,6-diazabicyclo[3.2.1]octane-2-carbohydrazide;

Sodium and Trifluoroacetic acid salt of (2S, 5R)-N-{[4-amino-2-oxopyrimidin-1(2H)-yl]acetyl}-7-oxo-6-(sulfooxy)-1,6-diazabicyclo[3.2.1]octane-2-carbohydrazide;

Sodium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide;

Mixture of sodium salt of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide and (2S,5R)-N′-{[5-(Hydroxymethyl)-1H-1,2,3-triazol-1-yl]acetyl}-7-oxo-6-(sulfooxy)-1,6-diazabicyclo[3.2.1]octane-2-carbohydrazide;

Sodium salt of (2S,5R)-N′-{[2-(hydroxymethyl)-1H-imidazol-1-yl]acetyl}-7-oxo-6-(sulfooxy)-1,6-diazabicyclo[3.2.1]octane-2-carbohydrazide;

Sodium salt of (2S,5R)-N′-[2-(ethoxycarbonyl)-1H-imidazol-1-ylacetyl]-7-oxo-6-(sulfooxy)-1,6-diazabicyclo[3.2.1]octane-2-carbohydrazide;

Sodium salt of (2S,5R)-N′-[2-(carbamoyl)-1H-imidazol-1-ylacetyl]-7-oxo-6-(sulfooxy)-1,6-diazabicyclo[3.2.1]octane-2-carbohydrazide;

Sodium salt of (2S,5R)-N′-[2-(methoxycarbonyl)-1H-imidazol-1-ylacetyl]-7-oxo-6-(sulfooxy)-1,6-diazabicyclo[3.2.1]octane-2-carbohydrazide;

Sodium salt of (2S,5R)-N-[2-[5-(aminomethyl)-2H-tetrazol-2-yl]ethoxy}-7-oxo-6-(sulfooxy)-1,6-diazabicyclo[3.2.1]octane-2-carboxamide;

Sodium salt of (2S,5R)-N-(2-(2H-1,2,3-triazol-2-yl)ethoxy)-6-(sulfooxy)-7-oxo-1,6-diaza-bicyclo[3.2.1]octane-2-carboxamide.

Sodium salt of (2S,5R)-N-(2-(1H-1,2,3-triazol-1-yl)ethoxy)-6-(sulfooxy)-7-oxo-1,6-diaza-bicyclo[3.2.1]octane-2-carboxamide;

Sodium salt of (2S,5R)-7-oxo-6-(sulfooxy)-N-[2-(1H-1,2,4-triazol-1-yl)ethoxy]-1,6-diazabicyclo[3.2.1]octane-2-carboxamide;

Sodium salt of (2S,5R)-N-[2-[5-(ethoxycarbonyl)-1H-tetrazol-1-yl]ethoxy}-6-sulfooxy-7-oxo-1,6-diazabicyclo[3.2.1]octane-2-carboxamide;

Sodium salt of (2S,5R)-7-oxo-6-(sulfooxy)-N-[2-(2H-tetrazol-2-yl)ethoxy]-1,6-diazabicyclo[3.2.1]octane-2-carboxamide;

Sodium salt of (2S,5R)-7-oxo-6-(sulfooxy)-N-[2-(1H-tetrazol-1-yl)ethoxy]-1,6-diazabicyclo[3.2.1]octane-2-carboxamide;

Sodium salt of (2S,5R)-N-[2-(5-methyl-1H-tetrazol-1-yl)ethoxy]-7-oxo-6-(sulfooxy)-1,6-diazabicyclo[3.2.1]octane-2-carboxamide;

Sodium salt of (2S,5R)-N-[2-(5-methyl-2H-tetrazol-2-yl)ethoxy]-7-oxo-6-(sulfooxy)-1,6-diazabicyclo[3.2.1]octane-2-carboxamide;

Sodium salt of (2S,5R)-N-[2-(3-amino-1H-1,2,4-triazol-1-yl)ethoxy]-7-oxo-6-(sulfooxy)-1,6-diazabicyclo[3.2.1]octane-2-carboxamide;

Sodium salt of (2S,5R)-N-[2-(5-amino-1H-tetrazol-1-yl)ethoxy]-7-oxo-6-(sulfooxy)-1,6-diazabicyclo[3.2.1]octane-2-carboxamide;

Sodium salt of (2S,5R)-N-[2-(5-amino-2H-tetrazol-2-yl)ethoxy]-7-oxo-6-(sulfooxy)-1,6-diazabicyclo[3.2.1]octane-2-carboxamide;

Sodium salt of (2S,5R)-N-[2-(5-carbamoyl-1H-tetrazol-1-yl)ethoxy]-7-oxo-6-(sulfooxy)-1,6-diazabicyclo[3.2.1]octane-2-carboxamide;

Sodium salt of (2S,5R)-N-[2-[5-(2-ethoxy-2-oxoethyl)-2H-tetrazol-2-yl]ethoxy}-6-sulfooxy-7-oxo-1,6-diazabicyclo[3.2.1]octane-2-carboxamide;

Sodium salt of (2S,5R)-N-[2-[5-(2-ethoxy-2-oxoethyl)-1H-tetrazol-1-yl]ethoxy}-6-(sulfooxy)-7-oxo-1,6-diazabicyclo[3.2.1]octane-2-carboxamide;

Sodium salt of (2S,5R)-N-[2-[5-(2-amino-2-oxoethyl)-2H-tetrazol-2-yl]ethoxy}-7-oxo-6-(sulfooxy)-1,6-diazabicyclo[3.2.1]octane-2-carboxamide;

Sodium salt of (2S,5R)-N-[2-[5-(2-amino-2-oxoethyl)-1H-tetrazol-1-yl]ethoxy}-7-oxo-6-(sulfooxy)-1,6-diazabicyclo[3.2.1]octane-2-carboxamide;

Sodium salt of (2S,5R)-N-[2-[5-(aminomethoxy)-1H-tetrazol-1-yl]ethoxy}-7-oxo-6-(sulfooxy)-1,6-diazabicyclo[3.2.1]octane-2-carboxamide;

Sodium salt of (2S,5R)-N-[2-[5-(2-aminoethoxy)-1H-tetrazol-1-yl]ethoxy}-7-oxo-6-(sulfooxy)-1,6-diazabicyclo[3.2.1]octane-2-carboxamide;

Sodium salt of (2S,5R)-N-[2-[5-(3-aminopropoxy)-1H-tetrazol-1-yl]ethoxy}-6-(sulfooxy)-7-oxo-1,6-diazabicyclo[3.2.1]octane-2-carboxamide;

Sodium salt of (2S,5R)-N-[2-[5-(2-amino-1-oxo-ethoxy)-1H-tetrazol-1-yl]ethoxy}-7-oxo-6-(sulfooxy)-1,6-diazabicyclo[3.2.1]octane-2-carboxamide;

Sodium salt of (2S,5R)-N-[2-[5-[2-aminoethyl)-2H-tetrazol-2-yl]ethoxy}-7-oxo-6-(sulfooxy)-1,6-diazabicyclo[3.2.1]octane-2-carboxamide;

Sodium salt of (2S,5R)-N-[2-[4-(hydroxymethyl)-2H-1,2,3-triazol-2-yl]ethoxy}-7-oxo-6-(sulfooxy)-1,6-diazabicyclo[3.2.1]octane-2-carboxamide;

Sodium salt of (2S,5R)-N-[2-[5-(2-hydroxyethyl)-2H-tetrazol-2-yl]ethoxy}-7-oxo-6-(sulfooxy)-1,6-diazabicyclo[3.2.1]octane-2-carboxamide;

Sodium salt of (2S,5R)-N-[2-[5-(2-hydroxyethyl)-1H-tetrazol-1-yl]ethoxy}-7-oxo-6-(sulfooxy)-1,6-diazabicyclo[3.2.1]octane-2-carboxamide;

Sodium salt of (2S,5R)-N-[2-[5-(2-oxo-2-ethoxyethyl)-2H-tetrazol-2-yl]ethoxy}-7-oxo-6-(sulfooxy)-1,6-diazabicyclo[3.2.1]octane-2-carboxamide;

or a stereoisomer thereof.

In general, the compounds of the invention can be prepared according to the general procedures given in Scheme 1 and Scheme 2. A person of skills in the art would appreciate that the described method can be varied or optimized further to provide the desired and related compounds. In the following procedures all variables are as defined above.

In one aspect, the compounds according to invention are prepared according to general procedure given in Scheme 1. A compound of Formula (III), Sodium salt of (2S, 5R)-6-benzyloxy-7-oxo-1,6-diaza-bicyclo[3.2.1]octane-2-carboxylic acid, (prepared as per the procedure disclosed in International Patent Application No. PCT/IB2013/059264) is treated with a compound of Formula (II) in presence of a suitable coupling agent and a suitable solvent at a temperature ranging from about −15° C. to about 60° C. for about 1 to about 24 hours to obtain a compound of Formula (IV). Typical, non-limiting examples of suitable coupling agents include dicyclohexylcarbodiimide (DCC), 1-ethyl-3-(3-dimethylaminopropyl) carbodiimide hydrochloride (EDC.HCl), diisopropylcarbodiimide (DIC), 1-hydroxybenzotriazole (HOBt), N-hydroxysuccinimide (HOsu), 1-Hydroxy-7-azabenzotriazolo (HOAt), (1-[B is(dimethylamino)methylene]-1H-1,2,3-triazolo[4,5-b]pyridinium-3-oxidhexafluorophosphate) (HATU), (benzotriazol-1-yl-oxytripyrrolidino phosphonium hexafluorophosphate) (PyBOP), Bromotripyrrolidinophosphonium hexafluorophosphate (PyBrop O-(7-azabenzotrizol-yl)-1,3-dimethyl-1,3-trimethylene uronium hexafluorophosphate (HAMTU), 2-(5-Norborene-2,3-dicarboximido)-1,1,3,3-tetramethyluronium tetrafluoroborate (TNTU), 2-(2-Pyridon-1-yl)-1,1,3,3-tetramethyluronium tetrafluoroborate (TPTU) and the like. Typical, non-limiting examples of suitable solvents include N,N-dimethylformamide, N,N-dimethylacetamide, 1,4-dioxane, chloroform, dichloromethane, tetrahydrofuran, acetonitrile, water and their appropriate combinations and the like.

The compound of Formula (IV) is debenzylated by carrying out hydrogenolysis in presence of hydrogen, transition metal catalyst and a suitable solvent at a temperature ranging from about 10° C. to about 60° C. for about 1 hour to about 14 hour to provide a compound of Formula (V). Typical, non-limiting examples of hydrogen source include hydrogen gas, ammonium formate, cyclohexene, lithium liquid ammonia, ammonia tert-butanol, sodium liquid ammonia tert-butanol, triethyl silyl hydride and the like. Typical, non-limiting examples of transition metal catalyst include 5% palladium on carbon, 10% palladium on carbon, 20% palladium hydroxide on carbon, Raney-Nickel and the like. Typical, non-limiting examples of suitable solvent include methanol, ethanol, dichloromethane, N,N dimethylformamide, ethyl acetate, tetrahydrofuran or a mixture thereof.

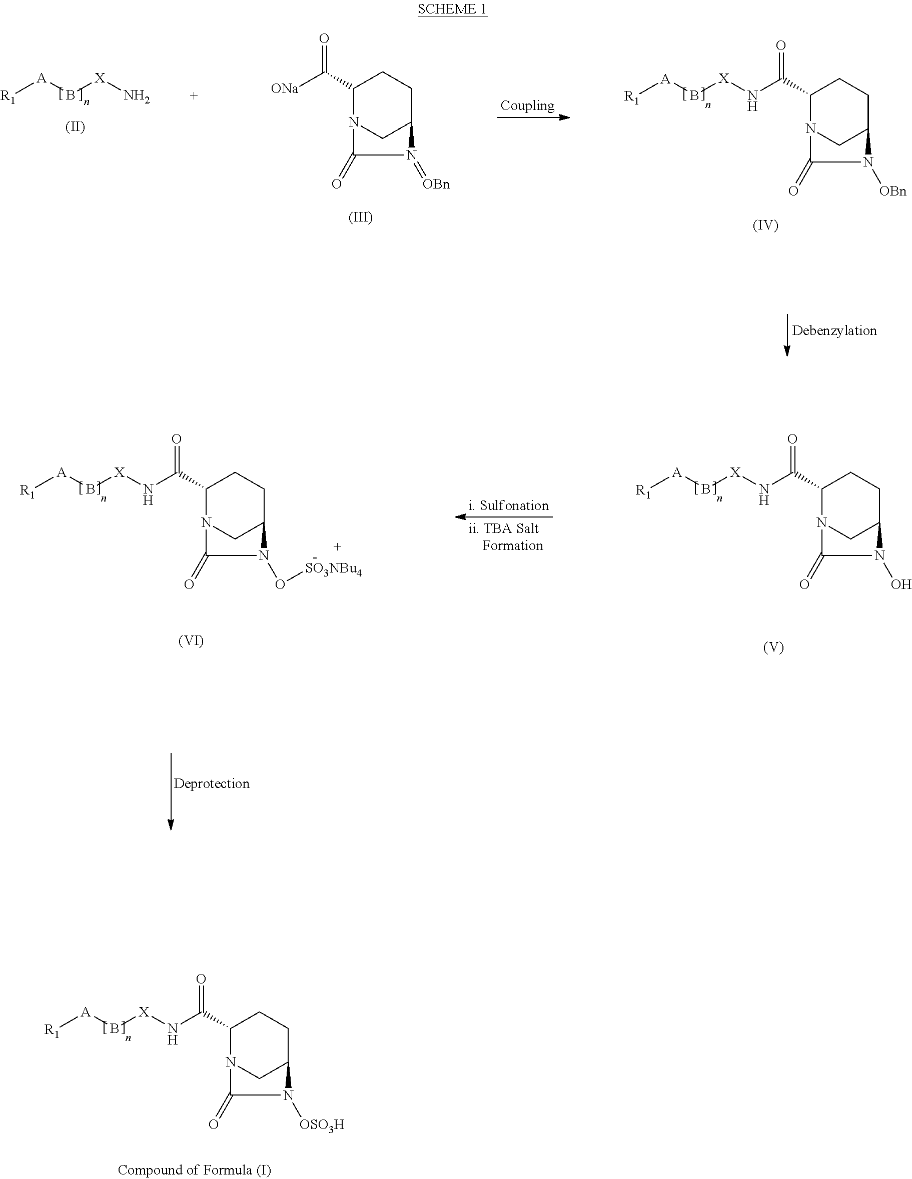

The compound of Formula (V) is sulfonated by reacting with suitable sulfonating reagent in a suitable solvent such as pyridine, dichloromethane or N,N-dimethylformamide, at a temperature ranging from about 0° C. to about 80° C. for about 1 hour to about 24 hour. Typical non-limiting examples of suitable sulfonating reagent include sulfur trioxide pyridine complex, sulfur trioxide trimethylamine complex, sulfur trioxide triethylamine complex, sulfur trioxide N,N-dimethylaniline complex, sulfur trioxide 2-methylpyridine complex, sulfur trioxide dioxane complex, sulfur trioxide thioxane complex, sulfur trioxide dimethyl sulfide complex, sulfur trioxide dimethylsulfoxide complex, sulfur trioxide N,N-dimethylformamide complex and the like. The obtained sulfonated compound was converted to corresponding tetrabutylammonium salt of Formula (VI). In some embodiments, the sulfonated compound is treated with tetrabutylammonium hydrogen sulfate (TBAHS) to obtain tetrabutylammonium salt of sulfonic acid compound of Formula (VI).

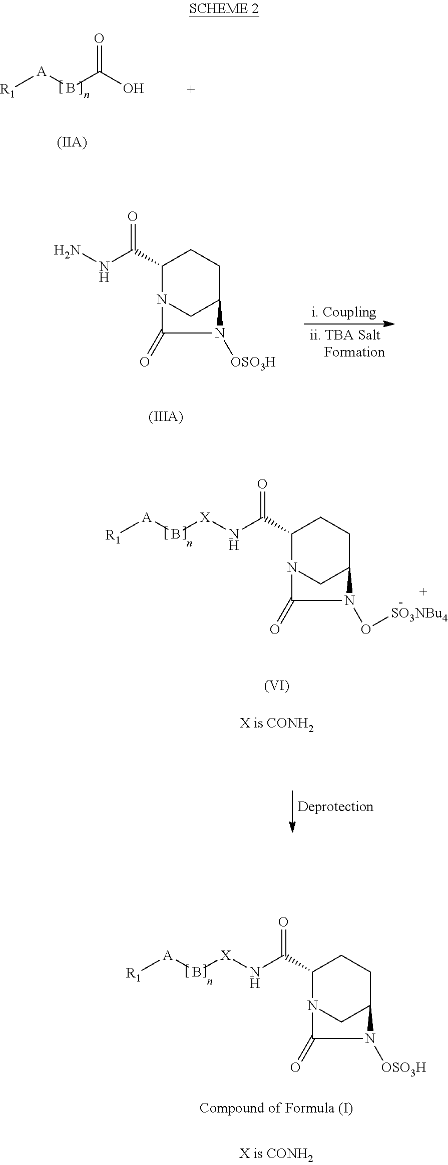

In some embodiments, compound of Formula (VI) is obtained according to procedure disclosed in Scheme 2. In some embodiments, the compound of Formula (IIA) is reacted with a compound of Formula (IIIA) to obtain a compound of Formula (VI). The compound of Formula (IIA) is coupled with a compound of Formula (IIIA), and this is followed by tetrabutylammonium salt formation to obtain a compound of Formula (VI).

The compound according to the invention is then isolated as zwitterions, by removing the protecting groups of compound of Formula (VI). The compound of Formula (VI) is reacted with suitable deprotecting agent such as trifluoroacetic acid in presence of a suitable solvent such as dichloromethane, chloroform or acetonitrile, at a temperature ranging from about −15° C. to about 40° C. for about 0.5 hours to about 14 hour to obtain a compound of Formula (I).

The compound of Formula (VI) may also be converted to pharmaceutically acceptable salts of compound of Formula (I). In some embodiments, compound of Formula (VI) was dissolved in suitable solvent such as 10% tetrahydrofuran: water mixture and was passed through the column packed with Dowex 50WX8 200 Na+ resin or passing through Indion 225 Na resin to provide sodium salt of compound of Formula (I). In some embodiments, compound of Formula (VI) was dissolved in suitable solvent such as acetone, tetrahydrofuran, ethanol, isopropanol or acetonitrile and thereby treating with sodium ethylhexanoate or potassium ethylhexanoate to provide sodium or potassium salt of compound of Formula (I).

In some embodiments, there are provided pharmaceutical compositions comprising a compound of Formula (I) or a stereoisomer or a pharmaceutically acceptable salt thereof.

In some other embodiments, there are provided pharmaceutical compositions comprising: (a) a compound of Formula (I) or a stereoisomer or a pharmaceutically acceptable salt thereof, and (b) at least one beta-lactamase inhibitor.

In some other embodiments, there are provided pharmaceutical compositions comprising: (a) a compound of Formula (I) or a stereoisomer or a pharmaceutically acceptable salt thereof, and (b) at least one beta-lactamase inhibitor selected from sulbactam, tazobactam, clavulanic acid or avibactam.

In some other embodiments, there are provided pharmaceutical compositions comprising: (a) a compound of Formula (I) or a stereoisomer or a pharmaceutically acceptable salt thereof, and (b) at least one antibacterial agent.

In some other embodiments, there are provided pharmaceutical compositions comprising: (a) a compound of Formula (I) or a stereoisomer or a pharmaceutically acceptable salt thereof, and (b) at least one antibacterial agent selected from cefepime, cefpirome, ceftaroline, ceftazidime or ceftalozane.

In some other embodiments, there are provided pharmaceutical compositions comprising: (a) a compound of Formula (I) or a stereoisomer or a pharmaceutically acceptable salt thereof, (b) at least one beta-lactamase inhibitor, and (c) at least one antibacterial agent.

In some other embodiments, there are provided methods for treating a bacterial infection in a subject, the methods comprising administering to the subject a pharmaceutical composition comprising a compound of Formula (I) or a stereoisomer or a pharmaceutically acceptable salt thereof.

In some other embodiments, there are provided methods for treating a bacterial infection in a subject, the methods comprising administering to the subject a pharmaceutical composition comprising: (a) a compound of Formula (I) or a stereoisomer or a pharmaceutically acceptable salt thereof and (b) at least one beta-lactamase inhibitor.

In some other embodiments, there are provided methods for treating a bacterial infection in a subject, the methods comprising administering to the subject a pharmaceutical composition comprising: (a) a compound of Formula (I) or a stereoisomer or a pharmaceutically acceptable salt thereof and (b) at least one beta-lactamase inhibitor selected from sulbactam, tazobactam, clavulanic acid or avibactam.

In some other embodiments, there are provided methods for treating a bacterial infection in a subject, the methods comprising administering to the subject a pharmaceutical composition comprising: (a) a compound of Formula (I) or a stereoisomer or a pharmaceutically acceptable salt thereof, and (b) at least one antibacterial agent.

In some other embodiments, there are provided methods for treating a bacterial infection in a subject, the methods comprising administering to the subject a pharmaceutical composition comprising: (a) a compound of Formula (I) or a stereoisomer or a pharmaceutically acceptable salt thereof, and (b) at least one antibacterial agent selected from selected from cefepime, cefpirome, ceftaroline, ceftazidime or ceftalozane.

In some other embodiments, there are provided methods for treating a bacterial infection in a subject, the methods comprising administering to the subject a pharmaceutical composition comprising: (a) a compound of Formula (I) or a stereoisomer or a pharmaceutically acceptable salt thereof, (b) at least one beta-lactamase inhibitor and (c) at least one antibacterial agent.

In some other embodiments, there are provided methods for treating a bacterial infection in a subject, said method comprising administering to the subject a compound of Formula (I) or a stereoisomer or a pharmaceutically acceptable salt thereof.

In some other embodiments, there are provided methods for treating a bacterial infection in a subject, the methods comprising administering to the subject: (a) a compound of Formula (I) or a stereoisomer or a pharmaceutically acceptable salt thereof, (b) at least one beta-lactamase inhibitor.

In some other embodiments, there are provided methods for treating a bacterial infection in a subject, the methods comprising administering to the subject: (a) a compound of Formula (I) or a stereoisomer or a pharmaceutically acceptable salt thereof, (b) at least one beta-lactamase inhibitor selected from sulbactam, tazobactam, clavulanic acid or avibactam.

In some other embodiments, there are provided methods for treating a bacterial infection in a subject, the methods comprising administering to the subject: (a) a compound of Formula (I) or a stereoisomer or a pharmaceutically acceptable salt thereof, (b) at least one antibacterial agent.

In some other embodiments, there are provided methods for treating a bacterial infection in a subject, the methods comprising administering to the subject: (a) a compound of Formula (I) or a stereoisomer or a pharmaceutically acceptable salt thereof, (b) at least one antibacterial agent selected from selected from cefepime, cefpirome, ceftaroline, ceftazidime or ceftalozane.

In some other embodiments, there are provided methods for treating a bacterial infection in a subject, the methods comprising administering to the subject: (a) a compound of Formula (I) or a stereoisomer or a pharmaceutically acceptable salt thereof, (b) at least one beta-lactamase inhibitor, and (c) at least one antibacterial agent.

In some embodiments, the compositions and methods according to the invention use compounds of Formula (I) or a stereoisomer or a pharmaceutically acceptable salt thereof in combination with at least one antibacterial agent. A wide variety of antibacterial agents can be used. Typical, non-limiting examples of antibacterial agents include one or more of antibacterial compounds generally classified as aminoglycosides, ansamycins, carbacephems, cephalosporins, cephamycins, lincosamides, lipopeptides, macrolides, monobactams, nitrofurans, penicillins, polypeptides, quinolones, sulfonamides, tetracyclines, oxazolidinone and the like. Typical, non-limiting examples of aminoglycoside antibacterial agents include amikacin, gentamicin, kanamycin, neomycin, netilmicin, tobramycin, paromomycin, arbekacin, streptomycin, apramycin and the like. Typical, non-limiting examples of ansamycin antibacterial agents include geldanamycin, herbimycin and the like. Typical, non-limiting examples of carbacephem antibacterial agents include loracarbef and the like. Typical, non-limiting examples of carbapenem antibacterial agents include ertapenem, doripenem, imipenem, meropenem and the like.

Typical, non-limiting examples of cephalosporin and cephamycin antibacterial agents include cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefmetazole, carbacephem, cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, cxacephem, cefepime, cefozopran, cefpirome, cefquinome, ceftobiprole, ceftiofur, cefquinome, cefovecin, ceftolozane, ceftaroline, ceftobiprole and the like.

Typical, non-limiting examples of lincosamide antibacterial agents include clindamycin, lincomycin and the like. Typical, non-limiting examples of macrolide antibacterial agents include azithromycin, clarithromycin, dirithromycin, erythromycin, roxithromycin, troleandomycin, telithromycin, spectinomycin, solithromycin and the like. Typical, non-limiting examples of monobactam antibacterial agents include aztreonam and the like. Typical, non-limiting examples of nitrofuran antibacterial agents include furazolidone, nitrofurantoin and the like. Typical, non-limiting examples of penicillin antibacterial agents include amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin and the like. Typical, non-limiting examples of polypeptide antibacterial agents include bacitracin, colistin, polymyxin B and the like.

Typical, non-limiting examples of quinolone antibacterial agents include ciprofloxacin, enoxacin, gatifloxacin, levofloxacin, lomefloxacin, moxifloxacin, nalidixic acid, levonadifloxacin, norfloxacin, ofloxacin, trovafloxacin, grepafloxacin, sparfloxacin, temafloxacin and the like. Typical, non-limiting examples of sulfonamide antibacterial agents include mafenide, sulfonamido chrysoidine, sulfacetamide, sulfadiazine, sulfamethizole, sulfametho xazole, sulfasalazine, sulfisoxazole, trimethoprim and the like. Typical, non-limiting examples of tetracycline antibacterial agents include demeclocycline, doxycycline, minocycline, oxytetracycline, tetracycline, tigecycline and the like. Typical, non-limiting examples of oxazolidinone antibacterial agents include tedizolid, linezolid, ranbezolid, torezolid, radezolid and the like.

The terms ‘antibacterial agent’ and ‘beta-lactamase inhibitor’ also includes their pharmaceutically acceptable derivative thereof (such as salts, pro-drugs, metabolites, esters, ethers, hydrates, polymorphs, solvates, complexes, and adducts) which, upon administration to a subject, are capable of providing (directly or indirectly) the parent compound.

The pharmaceutical compositions according to the invention may include one or more pharmaceutically acceptable carriers or excipients or the like. Typical, non-limiting examples of such carriers or excipient include mannitol, lactose, starch, magnesium stearate, sodium saccharine, talcum, cellulose, sodium crosscarmellose, glucose, gelatin, sucrose, magnesium carbonate, wetting agents, emulsifying agents, solubilizing agents, pH buffering agents, lubricants, stabilizing agents, binding agents etc.

In some embodiments, pharmaceutical compositions according to the present invention are administered orally or parenterally.

The pharmaceutical compositions according to the invention may be formulated into a variety of solid oral dosage forms. Typical, non-limiting examples of some oral dosage forms include tablet, capsule, powder, discs, caplets, pellets, granules, granules in capsule, minitablets, minitablets in capsule, pellets in capsule and the like. In some embodiments, the compositions according to invention may also be formulated into other dosage form suitable for oral administration such as suspensions, emulsions, syrups, elixirs and the like.

The pharmaceutical compositions according to this invention can exist in various forms. In some embodiments, the pharmaceutical composition is in the form of a powder or a solution. In some other embodiments, the pharmaceutical compositions according to the invention are in the form of a powder that can be reconstituted by addition of a compatible reconstitution diluent prior to parenteral administration. Non-limiting example of such a compatible reconstitution diluent includes water for injection, 0.9% sodium chloride solution and 5% dextrose solution.

In some other embodiments, the pharmaceutical compositions according to the invention are in the form of a frozen composition that can be diluted with a compatible diluent prior to parenteral administration.

In some other embodiments, the pharmaceutical compositions according to the invention are in the form ready to use for oral or parenteral administration.

In the methods according to the invention, the pharmaceutical composition and/or other pharmaceutically active ingredients disclosed herein may be administered by any appropriate method, which serves to deliver the composition or its constituents or the active ingredients to the desired site. The method of administration can vary depending on various factors, such as for example, the components of the pharmaceutical composition and nature of the active ingredients, the site of the potential or actual infection, the microorganism (e.g. bacteria) involved, severity of infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash.

The compositions according to the invention can be formulated into various dosage forms wherein the active ingredients and/or excipients may be present either together (e.g. as an admixture) or as separate components. When the various ingredients in the composition are formulated as a mixture, such composition can be delivered by administering such a mixture to a subject using any suitable route of administration. Alternatively, pharmaceutical compositions according to the invention may also be formulated into a dosage form wherein one or more ingredients (active or inactive ingredients) are present as separate components. The composition or dosage form wherein the ingredients do not come as a mixture, but come as separate components, such composition/dosage form may be administered in several ways. In one possible way, the ingredients may be mixed in the desired proportions and the mixture is then administered as required. Alternatively, the components or the ingredients (active or inert) may be separately administered (simultaneously or one after the other) in appropriate proportion so as to achieve the same or equivalent therapeutic level or effect as would have been achieved by administration of the equivalent mixture.

In some embodiments, pharmaceutical compositions according to the invention are formulated into a dosage form such that the compound of Formula (I) or a stereoisomer or a pharmaceutically acceptable salt thereof, and the antibacterial agent, are present in the composition as admixture or as separate components. In some other embodiments, pharmaceutical compositions according to the invention are formulated into a dosage form such that the compound of Formula (I) or a stereoisomer or a pharmaceutically acceptable salt thereof, and the antibacterial agent, are present in the composition as separate components.

Similarly, in the methods according to the invention, the active ingredients disclosed herein may be administered to a subject in several ways depending on the requirements. In some embodiments, the active ingredients are admixed in appropriate amounts and then the admixture is administered to a subject. In some other embodiments, the active ingredients are administered separately. Since the invention contemplates that the active ingredients agents may be administered separately, the invention further provides for combining separate pharmaceutical compositions in kit form. The kit may comprise one or more separate pharmaceutical compositions, each comprising one or more active ingredients. Each of such separate compositions may be present in a separate container such as a bottle, vial, syringes, boxes, bags, and the like. Typically, the kit comprises directions for the administration of the separate components. The kit form is particularly advantageous when the separate components are preferably administered in different dosage forms (e.g., oral and parenteral) ore are administered at different dosage intervals. When the active ingredients are administered separately, they may be administered simultaneously or sequentially.

In general, the pharmaceutical compositions and method disclosed herein are useful in treating bacterial infections. Advantageously, the compositions and methods disclosed herein are also effective in treating infections caused by bacteria that are considered be less or not susceptible to one or more of known antibacterial agents or their known compositions. Some non-limiting examples of such bacteria known to have developed resistance to various antibacterial agents include Acinetobacter, E. coli, Pseudomonas aeruginosa, Staphylococcus aureus, Enterobacter, Klebsiella, Citrobacter and a like. Other non-limiting examples of infections that may be prevented or treated using the compositions and/or methods of the invention include: skin and soft tissue infections, febrile neutropenia, urinary tract infection, intraabdominal infections, respiratory tract infections, pneumonia (nosocomial), bacteremia meningitis, surgical, infections etc.

Surprisingly, the compounds, compositions and methods according to the invention are also effective in treating bacterial infections that are caused by bacteria producing one or more beta-lactamase enzymes. In some embodiments, there are provided methods of inhibiting beta-lactamase enzymes, wherein the methods comprise administering a pharmaceutically effective amount of a compound of Formula (I) or a stereoisomer or a pharmaceutically acceptable salt thereof. In some other embodiments, there are provided methods of inhibiting beta-lactamase enzymes, wherein the methods comprise administering a pharmaceutically effective amount of a pharmaceutical composition comprising a compound of Formula (I) or a stereoisomer or a pharmaceutically acceptable salt thereof.

In general, the compounds of Formula (I) or a stereoisomer or pharmaceutically acceptable salt thereof according to invention are also useful in increasing antibacterial effectiveness of antibacterial agent in a subject. The antibacterial effectiveness of one or more antibacterial agents may be increased, for example, by co-administering said antibacterial agent with a pharmaceutically effective amount of a compound of Formula (I) or a stereoisomer or a pharmaceutically acceptable salt thereof according to the invention. In some embodiments, there is provided a method for increasing antibacterial effectiveness of the antibacterial agent in a subject, said method comprising co-administering said antibacterial agent with a compound of Formula (I) or a stereoisomer or a pharmaceutically acceptable salt thereof.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Preparation 1: Synthesis of Ethyl 1-(2-hydrazinyl-2-oxoethyl)-1H-tetrazole-5-carboxylate

Step 1: Synthesis of Ethyl 1-[2-(benzyloxy)-2-oxoethyl]-1H-tetrazole-5-carboxylate: To a 3 necked flask equipped with overhead stirrer was added a solution of ethyl 1H-tetrazole-5-carboxylate (20 g, 140.8 mmol) in dimethylformamide (200 ml) at 25-30° C. under stirring. To this potassium carbonate (21.38 g, 154.9 mmol) was added under continuous stirring, and effervescence was observed. Benzyl bromoacetate (29 g, 126.7 mmol) was added to it drop wise using addition funnel at 25-30° C. The progress of reaction was monitored by thin layer chromatography using mixture of chloroform and methanol (9:1) as solvent system. After complete consumption of starting material the reaction mixture was filtered through celite bed to remove insoluble inorganic material and then added to water (2000 ml) under stirring. The aqueous reaction mixture was extracted with ethyl acetate (3×500 ml). The organic extracts were combined and washed with brine (1×500 ml), dried over anhydrous sodium sulfate. The volatiles were removed under reduced pressure to yield 32 g of ethyl 1-[2-(benzyloxy)-2-oxoethyl]-1H-tetrazole-5-carboxylate in 78% yield.

Step 2: Synthesis of [5-(ethoxycarbonyl)-1H-tetrazol-1-yl]acetic acid: The solution of ethyl 1-[2-(benzyloxy)-2-oxoethyl]-1H-tetrazole-5-carboxylate (32 g, 110 mmol, product obtained in step 1) in tetrahydrofuran (700 ml) was added 10% palladium on carbon (6.4 g) and hydrogenated at 50 psi at ambient temperature. The progress of reaction was monitored by thin layer chromatography using mixture of chloroform and methanol (9:1) as solvent system. On complete consumption of starting material the reaction mixture was filtered on celite bed and washed with tetrahydrofuran (2×140 ml). The filtrate was concentrated under reduced pressure to provide 12 g of [5-(ethoxycarbonyl)-1H-tetrazol-1-yl]acetic acid in 54% yield.

Step 3: Synthesis of ethyl 1-(2-{2-[(benzyloxy)carbonyl]hydrazinyl}-2-oxoethyl)-1H-tetrazole-5-carboxylate: To a clean flask containing a solution of [5-(ethoxycarbonyl)-1H-tetrazol-1-yl]acetic acid (6 g, 30 mmol, product obtained in step 2) in dimethylformamide (60 ml) was added benzyl hydrazinecarboxylate (5 g, 30 mmol) at 25-30° C. under stirring. To this stirred solution EDC.HCl (8.59 g, 45 mmol) and hydroxy benzotriazole (4 g, 30 mmol) was added. Finally N-methyl morpholine (10 ml, 90 mmol) was added and the reaction mixture was stirred at ambient temperature. The progress of reaction was monitored by thin layer chromatography using mixture of chloroform and methanol (9:1) as solvent system. After complete consumption of starting material the reaction was quenched by adding it to water (600 ml) and then extracted with ethyl acetate (3×200 ml). The organic extracts were combined and washed with potassium hydrogen sulfate solution (1×250 ml), saturated sodium hydrogen carbonate solution (1×250 ml), brine (1×250 ml). The volatiles were removed under reduced pressure to provide 7.2 g of ethyl 1-(2-{2-[(benzyloxy)carbonyl]hydrazinyl}-2-oxoethyl)-1H-tetrazole-5-carboxylate in 68% yield.

Step 4: Synthesis of ethyl 1-(2-hydrazinyl-2-oxoethyl)-1H-tetrazole-5-carboxylate: To a solution of ethyl 1-(2-{2-[(benzyloxy)carbonyl[hydrazinyl}-2-oxoethyl)-1H-tetrazole-5-carboxylate (7.2 g, 20.6 mmol, product from Step 3) in methanol (70 ml) was added 10% palladium over carbon (720 mg). The solution was hydrogenated at 50 psi at 25-30° C. and progress of reaction was monitored by thin layer chromatography using mixture of chloroform and methanol (9:1) as solvent system. After complete consumption of starting material the reaction mixture was filtered through celite bed and washed with methanol (2×10 ml). The filtrate was concentrated under reduced pressure to provide 3.1 g of ethyl 1-(2-hydrazinyl-2-oxoethyl)-1H-tetrazole-5-carboxylate in 70% yield.

### Preparation 2: Synthesis of mixture of 2-(1H-1,2,3-triazol-1-yl)acetohydrazide and 2-(2H-1,2,3-triazol-2-yl) acetohydrazide

Step 1: Synthesis of mixture of ethyl 1H-1,2,3-triazol-1-ylacetate and ethyl 2H-1,2,3-triazol-2-ylacetate: To a suspension of potassium carbonate (55g, 0.398 mol) in ethanol (550 ml) was added 1H-[1,2,3]triazole (25 g, 0.361 mol) under stirring. A solution of ethyl bromoacetate (40 ml, 0.359 mol) in ethanol (40 ml) was slowly added under stirring and stirring continued for 90 hours. The insoluble solids formed were filtered and residue was washed with ethanol (20 ml). Filtrate was evaporated and the residue obtained was taken into ethyl acetate (250 ml) and organic layer was washed with water (125 ml) and brine (125 ml). The organic layer was collected and dried over anhydrous sodium sulfate and evaporated under reduced pressure to provide 33 g of mixture of ethyl 2-(1H-1,2,3-triazol-1-yl)acetate and ethyl 2-(2H-1,2,3-triazol-1-yl)acetate as semi solid mass in 59% yield.

Analysis:

Mass: 156.0 (M+1); for Molecular weight: 155 and Molecular formula: C₆H₉N₃O₂.

Step 2: Synthesis of mixture of 2-(1H-1,2,3-triazol-1-yl)acetohydrazide and 2-(2H-1,2,3-triazol-2-yl) acetohydrazide: To a clean flask containing mixture of ethyl 2-(1H-1,2,3-triazol-1-yl)acetate and ethyl 2-(2H-1,2,3-triazol-1-yl)acetate (20 g, 0.129 mole, product from step 1) in ethanol (100 ml) was added hydrazine hydrate (9.5 ml) and heated to 80° C. under stirring. After stirring for one hour at 80° C. the reaction mass cooled to 0-5° C. and ethanol (100 ml) was added, continued the stirring for 1 hr. The precipitated compound was filtered and washed with pre-cooled ethanol (50 ml). The obtained precipitates were dried at 40° C. for 2 hours to provide 11.2 g of mixture of 2-(1H-1,2,3-triazol-1-yl)acetohydrazide and 2-(2H-1,2,3-triazol-1-yl)acetohydrazide as a white solid in 62% yield.

Analysis:

Mass: 142.0 (M+1); for Molecular weight: 141 and Molecular formula: C₄H₇N₅O

### Preparation 3: Synthesis of 2-(1H-1,2,4-triazol-1-yl)acetohydrazide

Step 1: Synthesis of ethyl 1H-1,2,4-triazol-1-yl)acetate: To a solution of 1H-[1,2,4]triazole (20 g, 0.289 mol) in dimethylformamide (100 ml) was added potassium carbonate (60 g, 0.434 mol). A solution of ethyl bromoacetate (29 ml, 0.261 mol) in dimethylformamide (29 ml) was slowly added and continued stirring for 16 hours. It was filtered and residue was washed with dimethylformamide (20 ml). The filtrate was slowly poured into chilled water (800 ml) and the mixture was extracted with ethyl acetate (3×200 ml). The combined organic layer was washed with water (200 ml) and brine (200 ml). The organic layer was collected and dried over anhydrous sodium sulphate and evaporated under reduced pressure to provide 25 g of ethyl 1H-1,2,4-triazol-1-yl)acetate as oily mass in 56% yield.

Analysis:

Mass: 156.0 (M+1); for Molecular weight: 155 and Molecular formula: C₆H₉N₃O₂.

Step 2: Synthesis mixture 2-(1H-1,2,4-triazol-1-yl)acetohydrazide: To a solution of ethyl 1H-1,2,4-triazol-1-yl)acetate (10 g, 0.0645 mol) in ethanol (50 ml) was added hydrazine hydrate (4.8 ml) and heated to 80° C. The reaction mixture was stirred for 1 hour and cooled to 0-5° C. and 50 ml ethanol was added and stirring was continued for 1 hour. The precipitated compound was filtered and washed with pre-cooled ethanol (25 ml). The compound was dried at 40° C. for 2 hours to provide 6 g of 2-(1H-1,2,4-triazol-1-yl)acetohydrazide as a white solid in 67% yield.

Analysis:

Mass: 142.0 (M+1); for Molecular weight: 141 and Molecular formula: C₄H₇N₅O.

### Preparation 4: Synthesis of 2-(5-methyl-1H-tetrazol-1-yl)acetohydrazide

Step 1: Synthesis of ethyl (5-methyl-1H-tetrazol-1-yl)acetate and ethyl (5-methyl-2H-tetrazol-1-yl)acetate: To a 3 necked flask equipped with overhead stirrer was added solution of 5-methyl-1H-tetrazole (11 g, 0.1309 mol) in dimethylformamide (55 ml) at 25-30° C. To this was added potassium carbonate (19.87 g, 0.144 mol) under stirring and effervescence was observed. Ethyl bromoacetate (21.86 g, 0.1309 mol) was added to it drop-wise using addition funnel at 25-30° C. The progress of reaction was monitored by thin layer chromatography using a mixture of chloroform and methanol (9:1) as solvent. After complete consumption of starting material the reaction mixture was filtered through celite bed to remove insoluble inorganic material and washed with (2×50 ml) ethyl acetate. The filtrated was charged to water (550 ml). The aqueous reaction mixture was extracted with ethyl acetate (3×250 ml). The organic extracts were combined and washed with brine (1×250 ml), dried over anhydrous sodium sulfate. The volatiles were removed under reduced pressure to get mixture of ethyl (5-methyl-1H-tetrazol-1-yl)acetate and ethyl (5-methyl-1H-tetrazol-1-yl)acetate. The crude product was separated by column chromatography using 100-200 mesh size silica gel and mixture of hexane and ethyl acetate (1:1) as eluting solvent to get 21 g of pure regioisomers (9 g of ethyl-(5-methyl-2H-tetrazol-1-yl)acetate as nonpolar isomer and 13 g of ethyl-(5-methyl-1H-tetrazol-1-yl)acetate as polar isomer) in 94% yield.

Step 2: Synthesis of 2-(5-methyl-1H-tetrazol-1-yl)acetohydrazide: A clean flask was charged with ethyl (5-methyl-1H-tetrazol-1-yl)acetate as polar isomer (13 g, 0.0764 mol) dissolved in ethanol (130 ml). To this was charged Hydrazine hydrate (3.82 g, 0.0764 mol) under stirring at 25-30° C. The progress of reaction was monitored by thin layer chromatography using a mixture of hexane and ethyl acetate (1:1). After complete consumption of starting material the volatiles were removed under reduced pressure and the residue was stirred in 130 ml diethyl ether to get precipitates of required compound. After stirring for 30 minutes and the the precipitates were filtered to get 9 g of off-white solid as 2-(5-methyl-1H-tetrazol-1-yl)acetohydrazide in 75% yield.

### Preparation 5: Synthesis of 2-(5-methyl-2H-tetrazol-1-yl)acetohydrazide

To a clean flask was charged ethyl (5-methyl-2H-tetrazol-1-yl)acetate as non-polar isomer (9 g, 0.0529 mol, obtained from preparation 4, step 1) dissolved in ethanol (90 ml). To this was charged hydrazine hydrate (2.64 g, 0.052 mol) under stirring at 25-30° C. The progress of reaction was monitored by thin layer chromatography using mixture of hexane and ethyl acetate (1:1) as solvent. After complete consumption of starting material the volatiles were removed under reduced pressure and the residue was stirred in 90 ml diethyl ether to get precipitates of required compound After stirring for 30 minutes the obtained precipitates were filtered to get 6.5 g of 2-(5-methyl-2H-tetrazol-1-yl)acetohydrazide as off-white solid in 79% yield.

### Preparation 6: Synthesis of 2-(1H-Pyrazol-1-yl)acetohydrazide

Step 1: Synthesis of Ethyl-1H-Pyrazol-1-ylacetate: To a clear solution of pyrazole (10 g, 0.146 mol) in dimethylformamide (50 ml) at 25-30° C. was added potassium carbonate (24.35 g, 0.176 mol) under stirring, effervescence was observed. After 10 minutes ethyl bromo acetate (24.51 g, 0.146 moles) was added under stirring. The completion of reaction was monitored by performing thin layer chromatography using a mixture of chloroform and methanol (9:1) as a solvent. After completion of the reaction, reaction mass was filtered and filtrate was concentrated under reduced pressure. The crude product was purified by silica gel column (60-120 mesh) using a mixture of methanol and dichloromethane as an eluent. The fractions containing the product were collected and the collective fraction was distilled to provide 18.8 g of ethyl-1H-pyrazol-1-ylacetate as yellowish thick oil in 83.03% yield.

Step 2: Synthesis of 2-(1H-Pyrazol-1-yl)acetohydrazide: To a clean dry flask was charged ethyl 1H-pyrazol-1-ylacetate (3.4 g, 0.022 mol, product obtained in Step 1) in ethanol (34 ml). The hydrazine hydrate (1.21 g, 0.024 mol) was charged to it under stirring at 25-30° C. and progress of reaction was monitored by thin layer chromatography using mixture of chloroform and methanol (9:1) as solvent system. After complete consumption of starting material the volatiles were removed under reduced pressure to get yellowish residue. To this was added diethyl ether (100 ml) and stirred to get precipitates of required compound. The product was filtered and washed with ether (2×17 ml) to provide 2.2 g of 2-(1H-pyrazol-1-yl)acetohydrazide in 71.1% yield. The obtained product was used as such without any further purification.

### Preparation 7: Synthesis of ethyl [1-(2-hydrazinyl-2-oxoethyl)-1H-tetrazol-5-yl]acetate

Step 1: Synthesis of Benzyl ethyl 2,2′-(1H-tetrazole-1,5-diyl)diacetate and benzyl ethyl 2,2′-(2H-tetrazole-2,5-diyl)diacetate: To a solution of ethyl 1H-tetrazol-5-ylacetate (12.5 g, 80.12 mmol, prepared according to the procedure described in ChemMedChem, 10(11), 1875-1883, 2015) in dimethylformamide (125 ml) was added potassium carbonate (16.58 g, 1207 mmol) under stirring at 25-30° C. To this resulted reaction mass was added benzyl bromoacetate (18.34 g, 80.12 mmol) drop-wise at temperature 25-30° C. After completion of 16 hours of stirring at same temperature, the completion of the reaction was confirmed by thin layer chromatography (hexane: ethyl acetate 1:1). Then the resulted reaction mixture was filtered through celite bed to remove insoluble inorganic and poured on to water (2.5 L). The aqueous reaction mixture was extracted with ethyl acetate (3×500 ml). The organic extracts were combined and washed with brine (500 ml), dried over anhydrous sodium sulfate. The volatiles were removed under reduced pressure to get mixture of benzyl ethyl 2,2′-(1H-tetrazole-1,5-diyfldiacetate and benzyl ethyl 2,2′-(2H-tetrazole-2,5-diyl)diacetate (21 g). The two isomers were separated by using column chromatography (100-200 mesh size silica gel) eluting with hexane: ethyl acetate (1:1). The non-polar isomer as per thin layer chromatography, benzyl ethyl 2,2′-(2H-tetrazole-2,5-diyl)diacetate, was eluted at 10-15% ethyl acetate in hexane to obtain 7.5 g of the pure product. The polar isomer benzyl ethyl 2,2′-(1H-tetrazole-1,5-diyl)diacetate was eluted at 20-25% ethyl acetate in hexane to obtain 10.6 g of pure product.

Step 2: Synthesis of [5-(2-ethoxy-2-oxoethyl)-1H-tetrazol-1-yl]acetic acid: To a solution of benzyl ethyl 2,2′-(1H-tetrazole-1,5-diyl)diacetate (10.5 g, 34.53 mmol, polar isomer from step 1) in tetrahydrofuran (105 ml) was added 1.5 g 10% Pd/C and hydrogenated at 50 psi at ambient temperature. The progress of reaction was monitored by thin layer chromatography (chloroform: methanol, 9:1). On complete consumption of starting material the reaction mixture was filtered on celite bed and washed with tetrahydrofuran (2×15 ml). The filtrate was concentrated under reduced pressure and yielded 7 gram of [5-(2-ethoxy-2-oxoethyl)-1H-tetrazol-1-yl]acetic acid in 94% yield.

Step 3: Synthesis of benzyl 2-{[5-(2-ethoxy-2-oxoethyl)-1H-tetrazol-1-yl]acetyl}hydrazine carboxylate: To a solution of {5-(2-ethoxy-2-oxoethyl)-1H-tetrazol-1-yl]acetic acid (6 g, 28 mmol, obtained from step 2) in dimethylformamide (60 ml) was added benzyl hydrazinecarboxylate (4.65g, 28 mmol) at 25-30° C. followed by EDC.HCl (8 g, 42 mmol) and hydroxy benzotriazole monohydrate (4.28 g, 195.3 mmol) under stirring. Further, N-methyl morpholine (9.3 ml, 84 mmol) was added. After 16 hours, the thin layer chromatography (chloroform: methanol 9:1) confirmed completion of reaction. Then the reaction was quenched by adding water (600 ml) and extracted with ethyl acetate (3×150 ml). The organic extracts were combined and washed with KHSO₄ solution (100 ml), saturated NaHCO₃ solution (100 ml), brine (100 ml) and dried over anhydrous sodium sulfate. The volatiles were removed under reduced pressure to provide 10 g of benzyl 2-{[5-(2-ethoxy-2-oxoethyl)-1H-tetrazol-1-yl]acetyl}hydrazine carboxylate in 98% yield.

Step 4: Synthesis of ethyl [1-(2-hydrazinyl-2-oxoethyl)-1H-tetrazol-5-yl]acetate: To a solution of benzyl 2-{[5-(2-ethoxy-2-oxoethyl)-1H-tetrazol-1-yl]acetyl}hydrazine carboxylate (10 g, 27 mmol, product from step 3) in methanol (100 ml) was added 10% Pd/C (2.5 g). The solution was hydrogenated at 80 psi at 25-30° C. and progress of the reaction was monitored by thin layer chromatography (chloroform: methanol, 9:1). After the complete consumption of starting material the reaction mixture was filtered through celite bed and washed with methanol (2×10 ml). The filtrate was concentrated under reduced pressure to get 4.5 g of ethyl [1-(2-hydrazinyl-2-oxoethyl)-1H-tetrazol-5-yl]acetate in 71% yield.

Analysis:

Mass: 227.2 (M−1), 229.2 (M+1); for Molecular weight: 228 and Molecular formula: C₇H₁₂N₆O₃.

¹HNMR (400 MHz, DMSO-d₆): δ 9.55 (bs, 1H), 5.14 (s, 2H), 4.38 (bs, 2H), 4.19 (s, 2H), 4.14-4.08 (m, 2H), 1.21 (t, 3H).

### Preparation 8: Synthesis of Ethyl [2-(2-hydrazinyl-2-oxoethyl)-2H-tetrazol-5-yl]acetate

The Ethyl [2-(2-hydrazinyl-2-oxoethyl)-2H-tetrazol-5-yl]acetate was obtained by following the steps 2 to 4 described for Preparation-7 using non-polar isomer benzyl ethyl 2,2’-(2H-tetrazole-2,5-diyl)diacetate, from Step-1 of Preparation-7, yielded ethyl [2-(2-hydrazinyl-2-oxoethyl)-1H-tetrazol-5-yl]acetate.

Analysis:

Mass: 227.2 (M−1), 229.2 (M+1); for Molecular weight: 228 and Molecular formula: C₇H₁₂N₆O₃.

¹HNMR (400 MHz, DMSO-d₆): δ 9.60 (bs, 1H), 5.33 (s, 2H), 4.42 (bs, 2H), 4.14-4.09 (m, 2H), 4.04 (s, 2H), 1.20 (t, 3H).

### Preparation 9: Synthesis of 2-[1-(2-Hydrazinyl-2-oxoethyl)-1H-tetrazol-5-yl]acetamide

Step 1: Synthesis of 2-(1H-tetrazol-5-yl)acetamide: To a solution methanolic ammonia (500 ml) was added solid material of ethyl 1H-tetrazol-5-ylacetate (100 g, 641 mmol) at 0° C. under stirring in portion wise. After completion of the addition the reaction mixture was further continued to stir at ambient temperature. The progress of reaction was monitored by thin layer chromatography (chloroform: methanol, 9:1). After complete consumption of starting material the solid obtained was filtered on Buchner Funnel and washed with cold methanol (2×25 ml). The solid obtained was taken in 1N HCl (200 ml) stirred for 5 minutes and concentrated to half of the original volume under reduced pressure. The precipitates of amide obtained were collected by filtration on Buchner funnel and washed with water (2×25 ml). The product was dried till constant weight under reduced pressure to get off 67 g of 2-(1H-tetrazol-5-yl)acetamide as white solid in 82% yield.

Step 2: Synthesis of ethyl [5-(2-amino-2-oxoethyl)-1H-tetrazol-1-yl]acetate and ethyl [5-(2-amino-2-oxoethyl)-2H-tetrazol-2-yl]acetate: To a solution of 2-(1H-tetrazol-5-yl)acetamide (60 g, 472 mmol, product from step 1) in dimethylformamide (300 ml) was added potassium carbonate (130 g, 944 mmol) under stirring at room temperature. After completion of 15 minutes of stirring ethyl bromoacetate (55 ml, 472 mmol) was added dropwise at 10-15° C. and after 30 minutes of stirring at the same temperature the reaction mixture was allowed to stir at ambient temperature. The progress of reaction was monitored by thin layer chromatography (chloroform: methanol, 9:1). After complete consumption of starting material the inorganic solid was removed by filtration on Buchner Funnel and washed with ethyl acetate (2×50 ml). The volatiles were removed under reduced pressure and residue was taken in ethyl acetate (500 ml) and washed with water (2×250 ml). The organic extract was dried over anhydrous sodium sulfate and concentrated under reduced pressure to yield the crude product as mixture of isomers. The two isomers were separated by column chromatography (silica gel 100-200 mesh) using chloroform: methanol (9:1) as an eluent. The non-polar isomer ethyl-[5-(2-amino-2-oxoethyl)-2H-tetrazol-2-yl[acetate was isolated using 4% methanol in chloroform as eluent to obtain 28 g of the product. The polar isomer ethyl-[5-(2-amino-2-oxoethyl)-1H-tetrazol-1-yl]acetate was isolated by using 5% methanol in chloroform, to obtain 30 g of the product.

Step 3: Synthesis of 2-[1-(2-hydrazinyl-2-oxoethyl)-1H-tetrazol-5-yl]acetamide: To a stirred of ethyl [5-(2-amino-2-oxoethyl)-1H-tetrazol-1-yl]acetate (23 g, 107.9 mmol) in ethanol (230 ml) was added hydrazine hydrate (5.4 ml, 107.9 mmol) at 25-30° C. under stirring. The reaction was monitored by TLC (chloroform: methanol, 9:1). After complete consumption of starting material the precipitates obtained were filtered on Buchner funnel and washed with cold ethanol (2×25 ml). The product was dried under reduced pressure till constant weight to yield 21.4 g of 2-[1-(2-hydrazinyl-2-oxoethyl)-1H-tetrazol-5-yl]acetamide as white solid in 95% yield.

Analysis:

Mass: 198.1 (M−1), 200.2 (M+1); for Molecular weight: 199 and Molecular formula: C₅H₉N₇O₂.

¹H NMR (DMSO-d₆): δ 9.55(bs, 1H), 7.78 (s, 1H), 7.31 (s, 1H), 5.15 (s, 2H), 4.37 (s, 2H), 3.94 (s, 2H).

### Preparation 10: Synthesis of 2-[2-(2-Hydrazinyl-2-oxoethyl)-2H-tetrazol-5-yl]acetamide

The 2-[2-(2-Hydrazinyl-2-oxoethyl)-2H-tetrazol-5-yl]acetamide was prepared by using procedure described in Step-3 of Preparation-9 using ethyl [5-(2-amino-2-oxoethyl)-2H-tetrazol-2-yl]acetate as a starting material (Step-2 product from Preparation-9).

Analysis:

Mass: 198.1 (M−1), 200.2 (M+1); for Molecular weight: 199 and Molecular formula: C₅H₉N₇O₂.

¹H NMR (DMSO-d₆): δ 9.59 (bs, 1H), 7.60 (s, 1H), 7.12 (s, 1H), 5.31 (s, 2H), 4.41 (s, 2H), 3.75 (s, 2H).

### Preparation 11: Synthesis of 2-(2-Hydrazinyl-2-oxoethyl)-2H-tetrazole-5-carboxamide

The 2-(2-Hydrazinyl-2-oxoethyl)-2H-tetrazole-5-carboxamide was synthesized by using the procedures described in Step-1 to Step-3 of Preparation-9 using ethyl 1H-tetrazole-5-carboxylate as starting material.

Analysis:

Mass: 184.1 (M−1) and 186.1(M+1); for Molecular weight: 185 and Molecular formula: C₄H₇N₇O₂.

¹HNMR (DMSO-d₆): δ 9.46 (bs, 1H), 8.68 (bs, 1H), 8.25 (bs, 1H), 5.39 (s, 2H), 4.37 (bs, 2H).

### Preparation 12: Synthesis of 1-(2-Hydrazinyl-2-oxoethyl)-1H-tetrazole-5-carboxamide

The 1-(2-Hydrazinyl-2-oxoethyl)-1H-tetrazole-5-carboxamide was synthesized by using the procedures described in Step-1 to Step-3 of Preparation-9 using ethyl 1H-tetrazole-5-carboxylate as starting material.

Analysis:

Mass: 184.1 (M−1) and 186.1(M+1); for Molecular weight: 185 and Molecular formula: C₄H₇N₇O₂.

¹H NMR (DMSO-d₆): δ 8.89 (bs, 1H), 8.63 (bs, 1H), 8.20 (bs, 1H), 5.70(s, 2H), 4.60 (bs, 2H).

### Preparation 13: Procedure for synthesis of 2-(1H-pyrrol-1-yl)acetohydrazide

To a solution ethyl 1H-pyrrol-1-ylacetate (11 g, 71.89 mmol, prepared according to the procedure described in Journal of Organic Chemistry, 79(3), 1368-1376; 2014) in ethanol (110 ml) was added hydrazine hydrate (3.6 g, 71.89 mmol) under stirring at ambient temperature. The progress of reaction was monitored by thin layer chromatography (chloroform: methanol, 9:1). After complete consumption of starting material the precipitates obtained were filtered on Buchner funnel and washed with ethanol (2×10 ml). The product was dried under reduced pressure till constant weight to get 2-(1H-pyrrol-1-yl)acetohydrazide as 4.6 g of off-white solid in 46% yield.

Analysis:

Mass: 138.0 (M−1), 140.1 (M+1); for Molecular weight: 139 and Molecular formula: C₆H₉N₃O.

¹H NMR (DMSO-d₆): δ 9.20 (bs, 1H), 6.69 (m, 2H), 5.98 (m, 2H), 4.43 (s, 2H), 4.28 (s, 2H).

### Preparation 14: Synthesis of (2-Methyl-imidazol-1-yl)-acetic acid hydrazide

To a solution of (2-methyl-imidazol-1-yl)-acetic acid ethyl ester (10.0 g, 59.52 mmol, prepared according to the procedure described in US 200505154024) in ethanol (100 ml) was added hydrazine hydrate (4.46 g, 89.28 mmol) at 25-30° C. under stirring and progress of reaction was monitored by t (chloroform: methanol, 9:1). After complete consumption of starting material the volatiles were removed under reduced pressure yielded yellowish residue. To this was added diethyl ether (50 ml) under stirring, the precipitates were formed during stirring were filtered and washed with ether (2×40 ml) to yield 8.0 g of (2-methyl-imidazol-1-yl)-acetic acid hydrazide as off-white solid in 87% yield.

Analysis:

Mass: 155.1 (M+1); for Molecular weight: 154 and Molecular formula: C₆H₁₀N₄O.

¹H NMR (DMSO-d₆): δ 9.34(s, 1H), 6.96 (s, 1H), 6.68 (s, 1H), 4.48 (s, 2H), 4.32 (s, 2H), 2.21 (s, 3H).

### Preparation 15: Synthesis of (2S)-2(1H-imidazol-1-yl)propanehydrazide

To a clean dry flask containing ethyl (2S)-2-(1H-imidazol-1-yl)propanoate (5 g, 29 mmol, prepared according to the procedure disclosed in Journal of Organic Chemistry, 68(2), 591-593, 2003) in ethanol (50 ml) was added hydrazine hydrate (1.48 ml, 29 mmol) under stirring 25-30° C. The progress of reaction was monitored by thin layer chromatography (TLC) (chloroform: methanol 9:1). After complete consumption of starting material the precipitates were filtered on Buchner funnel and washed with cold ethanol (2×10 ml). The product was dried under reduced pressure till constant weight to yield (2S)-2-(1H-imidazol-1-yl)propanehydrazide as off-white solid.

Analysis:

Mass: 153.0 (M−1); for Molecular weight: 154 and Molecular formula: C₆H₁₀N₄O.

¹H NMR (DMSO-d₆): δ 9.42 (brs, 1H), 7.64 (s, 1H), 7.17 (s, 1H), 6.86 (s, 1H), 4.79 (q, 1H, J=7.2 Hz), 4.31 (brs, 2H), 1.53 (d, 3H, J=6.8 Hz).

### Preparation 16: Synthesis of ethyl 1-(2-hydrazinyl-2-oxoethyl)-1H-imidazole-2-carboxylate

Step 1: Synthesis of ethyl 1-[2-(benzyloxy)-2-oxoethyl]-1H-imidazole-2-carboxylate: To a solution ethyl 1H-imidazole-2-carboxylate (6 g, 42.8 mmol, prepared as per the procedure depicted in Synthetic communication, 44(7), 968-975, 2014) in DMF (30 ml) at 25-30° C. followed by potassium carbonate (7.1 g, 51.3 mmol) under stirring. After 15 minutes of stirring benzyl bromoacetate (9.8 g, 42.8 mmol) was added drop-wise at same temperature. The progress of reaction was monitored by TLC (methanol: DCM, 1:9). After complete consumption of starting material the reaction mixture was filtered through celite bed to remove insoluble inorganic and poured on to water (150 ml). The aqueous reaction mixture was extracted with dichloromethane (3×60 ml). The organic extracts were combined and washed with brine (60 ml), dried over anhydrous sodium sulphate. The volatiles were removed under reduced pressure to yield 12 g of yielded ethyl 1-[2-(benzyloxy)-2-oxoethyl]-1H-imidazole-2-carboxylate in 97% yield.

Step 2: Synthesis of [2-(ethoxycarbonyl)-1H-imidazol-1-yl]acetic acid: To the solution of ethyl 1-[2-(benzyloxy)-2-oxoethyl]-1H-imidazole-2-carboxylate (12 g, 41.6 mmol, the product from step 1) in methanol (120 ml) was added 3 g 10% Pd/C and hydrogenated at 50 psi. The progress of reaction was monitored by TLC (chloroform: methanol, 9:1). On complete consumption of starting material the reaction mixture was filtered on celite bed and washed with methanol (2×25 ml). The filtrate was concentrated under reduced pressure to get 7 g of the desired compound [2-(ethoxycarbonyl)-1H-imidazol-1-yl]acetic acid in 94% yield.

Step 3: Synthesis of ethyl 1-(2-{2-[(benzyloxy)carbonyl]hydrazinyl1-2-oxoethyl)-1H-imidazole-2-carboxylate: To a solution of [2-(ethoxycarbonyl)-1H-imidazol-1-yl]acetic acid (7 g, 35.3 mmol) in dimethylformamide (70 ml) was charged benzyl hydrazinecarboxylate (5.86 g, 35.3 mmol) at 25-30° C. followed by EDC.HCl (10.11 g, 52.9 mmol), hydroxy benzotriazole monohydrate (5.94 g, 38.8 mmol) and finally N-methyl morpholine (11.64 ml, 105.9 mmol) was added under continuous stirring. The progress of reaction was monitored by TLC (chloroform:methanol, 9:1). After complete consumption of starting material the reaction mixture was distilled out on rotavapour till dryness. The reaction mass was partitioned in dichloromethane (70 ml) and water (70 ml). Aqueous layer was re-extracted with dichloromethane (2×70 ml). The organic extracts were combined and washed with brine (70 ml) and dried over anhydrous sodium sulfate. The volatiles were removed under reduced pressure to yield 8 g of ethyl 1-(2-{2-[(benzyloxy)carbonyl]hydrazinyl}-2-oxo ethyl)-1H-imidazole-2-carboxylate in 65% yield.

Step 4: Synthesis of ethyl 1-(2-hydrazinyl-2-oxoethyl)-1H-imidazole-2-carboxylate: To a solution of ethyl 1-(2-{2-[(benzyloxy)carbonyl]hydrazinyl}-2-oxoethyl)-1H-imidazole-2-carboxylate (8 g, 23 mmol, product from step 3) in methanol (80 ml) was added 10% Pd/C (2.0 g). The solution was hydrogenated at 50 psi at 25-30° C. and progress of reaction was monitored by TLC (chloroform:methanol, 9:1). After complete consumption of starting material the reaction mixture was filtered through celite bed and washed with methanol (2×16 ml). The filtrate was concentrated under reduced pressure to yield 4 g of ethyl 1-(2-hydrazinyl-2-oxoethyl)-1H-imidazole-2-carboxylate in 82% yield.

Analysis:

Mass: 213.2 (M+1); for Molecular weight: 212.21 and Molecular formula: C₈H₁₂N₄O₃.

### Preparation 17: Synthesis of 1-(2-hydrazinyl-2-oxoethyl)-1H-imidazole-2-carboxamide

Step 1: Synthesis of benzyl 2-[(2-carbamoyl-1H-imidazol-1-yl)acetyl]hydrazinecarboxylate: To a stirred solution of ethyl 1-(2-{2-[(benzyloxy)carbonyl]hydrazinyl}-2-oxoethyl)-1H-imidazole-2-carboxylate (8.0 g, 23 mmol, step 3 product of preparation 16) in methanol (80 ml) was added aqueous ammonia solution (40 ml). The progress of reaction was monitored by TLC (chloroform: methanol, 9:1). On complete consumption of starting material the methanol was distilled under vacuum. The solid thus obtained was filtered on Buchner funnel and washed with water (20 ml), dried under vacuum, to yield 7.5 g of benzyl 2-[(2-carbamoyl-1H-imidazol-1-yl)acetyl]hydrazinecarboxylate in 100% yield.

Step 2: Synthesis of 1-(2-hydrazinyl-2-oxoethyl)-1H-imidazole-2-carboxamide: To a solution of benzyl 2-[(2-carbamoyl-1H-imidazol-1-yl)acetyl]hydrazinecarboxylate (7.3 g, 23 mmol, product from step 1) in water (80 ml) was added 10% Pd/C (1.82 g). The solution was hydrogenated at 50 psi at 25-30° C. and progress of reaction was monitored by TLC (chloroform:methanol, 9:1). After complete consumption of starting material the reaction mixture was filtered through celite bed and washed with methanol (2×15 ml). The filtrate was concentrated under reduced pressure to yield 2.3 g of ethyl 1-(2-hydrazinyl-2-oxoethyl)-1H-imidazole-2-carboxamide in 55% yield.

Analysis:

Mass: 182.1(M−1), 184.1(M+1); for Molecular weight: 183.17 and Molecular formula: C₆H₉N₅O₂.

¹HNMR (DMSO-d₆): δ 9.51 (bs, 1H), 7.50 (bs, 1H), 7.27 (s, 1H), 7.09 (bs, 1H), 6.93 (s, 1H), 5.06 (s, 2H), 4.41(bs, 2H).

### Preparation 18: Synthesis of methyl 1-(2-hydrazinyl-2-oxoethyl)-1H-imidazole-2-carboxylate

Methyl 1-(2-hydrazinyl-2-oxoethyl)-1H-imidazole-2-carboxylate was prepared according to procedure described in preparation 16.

Analysis:

Mass: 199 (M+1); Molecular formula: C₇H₁₀N₄O₃, Molecular weight: 198.18.

### Preparation 19: Synthesis of tert-butyl {[1-(2-hydrazinyl-2-oxoethyl)-1H-tetrazol-5-yl]methyl}carbamate

Step 1: Synthesis of tert-butyl (cyanomethyl)carbamate: To a solution of hydrochloride salt of amino acetonitrile (50 g 487 mmol) dissolved in water (250 ml) and tetrahydrofuran (250 ml) was added sodium bicarbonate (90.14 g, 1073 mmol) under stirring at 25-30° C. To this was added di-tert-butyldicarbonate (117 g, 536 mmol) drop-wise using addition funnel. The progress of reaction was monitored by thin layer chromatography using a mixture of hexane and acetone as solvent. After complete consumption of starting material, ethyl acetate (500 ml) was added to it and organic layer was separated. The aqueous layer was re-extracted with ethyl acetate (1×250 ml). The organic extracts were combined and dried over anhydrous sodium sulfate, then concentrated to yield 66 g of tert-butyl (cyanomethyl)carbamate as a thick liquid in 86% yield.

Step 2: tert-butyl (1H-tetrazol-5-ylmethyl)carbamate and tert-butyl (2H-tetrazol-5-ylmethyl)carbamate: To a stirred solution of tert-butyl (cyanomethyl)carbamate (66 g, 423 mmol, product from step 1) dissolved in dimethylformamide (660 ml) was added triethylamine hydrochloride (174.5 g, 1269 mmol) and sodium azide (86 g, 1269 mmol) at 25-30° C. under stirring. Then the reaction mixture was heated at 80° C. and progress of reaction was monitored by thin layer chromatography using chloroform and methanol (9:1) as a solvent. After complete consumption of starting material the reaction mixture was brought to ambient temperature; filtered through celite bed and washed with dimethylformamide (2×25 ml). The volatiles were removed under reduced pressure and residue obtained was stirred in cold water (2000 ml) under vigorous stirring. The formed precipitates were filtered on Buchner funnel and washed with water (2×200 ml). The solid was collected and dried under reduced pressure till constant weight, to provide 70 g of a mixture of tert-butyl (1H-tetrazol-5-ylmethyl)carbamate and tert-butyl (2H-tetrazol-5-ylmethyl)carbamate in 83% yield.

Step 3: Synthesis of ethyl (5-{(tert-butoxycarbonyl)amino]methyl}-1H-tetrazol-1-yl)acetate and ethyl (5-{(tert-butoxycarbonyl)amino]methyl}-2H-tetrazol-2-yl)acetate: To a clear solution of tert-butyl (1H-tetrazol-5-ylmethyl)carbamate and tert-butyl (2H-tetrazol-5-ylmethyl)carbamate (25 g, 125 mmol, product from step 2) in N,N′-dimethylformamide (125 ml) was added potassium carbonate (19 g, 138 mmol) at 25-30° C. under stirring, effervescence was observed. After 10 minutes, ethyl bromoacetate (13 ml, 113 mmol) was added and the reaction mixture was stirred for overnight. The completion of the reaction was confirmed by performing thin layer chromatography using mixture of chloroform and methanol (9:1). After completion of the reaction, the reaction mass was added to water (1500 ml) and extracted with ethyl acetate (2×500 ml). The organic extracts were combined and washed with brine (1×500 ml), dried over anhydrous sodium sulfate, then concentrated under reduced pressure to provide 28 g of mixture of ethyl (5-{[(tert-butoxycarbonyl)amino]methyl}-1H-tetrazol-1-yl)acetate and ethyl (5-{(tert-butoxycarbonyl)aminolmethyl}-1H-tetrazol-1-yl)acetate as a reddish thick oil in 78% yield.

Step 4: Synthesis of tert-butyl {[1-(2-hydrazinyl-2-oxoethyl)-1H-tetrazol-5-yl]methyl}carbamate and tert-butyl {[1-(2-hydrazinyl-2-oxoethyl)-2H-tetrazol-5-yl]methyl}carbamate: To a clean dry flask was charged mixture of ethyl (5-{[(tert-butoxycarbonyl)amino]methyl}-1H-tetrazol-1-yl)acetate and ethyl (5-{[(tert-butoxycarbonyl) amino]methyl}-1H-tetrazol-1-yl)acetate. (20 g, 70 mmol, product from step 3) dissolved in ethanol (200 ml). To this solution was charged hydrazine hydrate (3.5 ml, 70.1 mmol) under stirring at 25-30° C. The white precipitate was seen after 30 minutes of addition of hydrazine hydrate. The progress of the reaction was monitored by thin layer chromatography using mixture of chloroform and methanol (9:1) as solvent. After complete consumption of starting material the precipitates were filtered and washed with ethanol (2×20 ml) to get 6.5 g of pure tert-butyl {[1-(2-hydrazinyl-2-oxoethyl)-1H-tetrazol-5-yl]methyl}carbamate as off white solid, which was used without further purification in the next step.

### Preparation 20: Synthesis of tert-butyl {[1-(2-hydrazinyl-2-oxoethyl)-2H-tetrazol-5-yl]methyl}carbamate

The mixture containing tert-butyl {[1-(2-hydrazinyl-2-oxoethyl)-1H-tetrazol-5-yl]methyl}carbamate and tert-butyl {[1-(2-hydrazinyl-2-oxoethyl)-2H-tetrazol-5-yl]methyl}carbamate (product from Preparation-19 of Step-4) was suspended in ethanol and filtered to yield the filtrate containing tert-butyl {[1-(2-hydrazinyl-2-oxoethyl)-2H-tetrazol-5-yl]methyl}carbamate. Upon concentration to dryness yielded a product containing the product in major was used without further purification in the next step.

### Preparation 21: Synthesis of tert-butyl 1-(2-hydrazino-2-oxoethyl)-1H-1,2,4-triazol-3-ylcarbamate

Step 1: Synthesis of ethyl {3-[(tert-butoxycarbonyl)amino]-1H-1,2,4-triazol-1-yl}acetate: To solution of ethyl (3-amino-1H-1,2,4-triazol-1-yl)acetate (15.5 g, 19.1 mmol, prepared according to the procedure given in WO2006066778) in dichloromethane (155 ml) was slowly added di-tert-butyldicarbonate (25 ml, 19.1 mmol) and dimethylamino pridine (1 mmol) at room temperature under nitrogen atmosphere and stirring. After completion of the 16 hours the reaction was found to be incomplete as depicted by thin layer chromatography. In order to achieve completion of the reaction triethylamine (16 ml, 11.4 mmol) was added and stirred further for 6 hours. The reaction mixture was washed with 10% ammonium chloride solution (100 ml) and water (100 ml). The organic layer was dried over anhydrous sodium sulfate and evaporated under reduced pressure to yield 14.6 g of ethyl {3-[(tert-butoxycarbonyl)amino]-1H-1,2,4-triazol-1-yl}acetate as oily crude mass in 59% yield.

Analysis:

Mass: 271.3 (M+1); for Molecular weight: 270 and Molecular formula: C₁₁H₁₈N₄O₄.

Step 2: Synthesis tert-butyl 1-(2-hydrazino-2-oxoethyl)-1H-1,2,4-triazol-3-ylcarbamate: To a solution of ethyl {3-[(tert-butoxycarbonyl)amino]-1H-1,2,4-triazol-1-yl}acetate (14 g, 5.18 mmol, from step 1) in dichloromethane (140 ml) was added hydrazine hydrate (3.2 ml, 6.55 mmol) under stirring at room temperature. After 2 hours, the completion of the reaction was monitored by thin layer chromatography using mixture of chloroform and methanol (9:1) as solvent. After complete conversion, precipitated solid was filtered out and filtrate was concentrated on rota evaporator under reduced pressure to obtain 11.2 g of crude tert-butyl 1-(2-hydrazino-2-oxoethyl)-1H-1,2,4-triazol-3-ylcarbamate as oily compound in 84% yield.

### Preparation 22: Synthesis of tert-butyl {2-[1-(2-hydrazinyl-2-oxoethyl)-1H-tetrazol-5-yl]ethyl}carbamate

Step 1: Synthesis of ethyl (5-{2-[(tert-butoxycarbonyl)amino]ethyl}-1H-tetrazol-1-yl)acetate and ethyl (5-{2-[(tert-butoxycarbonyl)amino]ethyl}-2H-tetrazol-2-yl)acetate: To a solution of tert-butyl [2-(1H-tetrazol-5-yl)ethyl]carbamate (24 g, 112 mmol, prepared according to the procedure described in Asian Journal of Chemistry, 22(7), 5165-5174, 2010) in dimethylformamide (240 ml) at 25-30° C. was added potassium carbonate (15.54 g, 112 mmol) under stirring. Then the reaction mass was cool to 10-15° C. and then added ethyl bromoacetate (12.54 ml, 112 mmol) using addition funnel under stirring. Stirring continued further and progress of reaction was monitored by TLC (chloroform: methanol, 9:1). After complete consumption of starting material the reaction was quenched by adding reaction mixture to water (2.4 L) and extracted with ethyl acetate (2×500 ml). The organic extracts were combined and dried over anhydrous sodium sulfate and concentrated under reduced pressure to yield a mixture of isomers. These isomers were separated by column chromatography (silica gel 100:200 mesh size) using methanol in chloroform as an eluent. The non-polar isomer ethyl (5-{2-[(tert-butoxycarbonyl)amino]ethyl}-2H-tetrazol-2-yl)acetate was isolated using at 2-3% methanol in chloroform in 10 g yield and the polar isomer ethyl (5-{2-[(tert-butoxycarbonyl)amino]ethyl}-1H-tetrazol-1-yl)acetate was isolated at 4% methanol in chloroform, in 13 g yield.

Step 2: Synthesis of tert-butyl {2-[1-(2-hydrazinyl-2-oxoethyl)-1H-tetrazol-5-yl]ethyl}carbamate: To a solution of ethyl (5-{2-[(tert-butoxycarbonyl)amino]ethyl}-1H-tetrazol-1-yl)acetate (10 g, 33.44 mmol) in ethanol (100 ml) was added hydrazine hydrate (1.67 ml, 33.44 mmol) under stirring. The progress of reaction was monitored by TLC (chloroform:methanol 9:1). The precipitates obtained were filtered on Buchner funnel and washed with Ethanol (2×25 ml). The product was dried under reduced pressure till constant weight to get 8 g of tert-butyl {2-[1-(2-hydrazinyl-2-oxoethyl)-1H-tetrazol-5-yl]ethyl}carbamate as off-white solid in 84% yield.

Analysis:

Mass: 284. 2 (M−1), 286.2 (M+1); for Molecular weight: 285 and Molecular formula: C₁₀H₁₉N₇O₃.

¹HNMR (DMSO-d₆): δ 9.50 (bs, 1H), 6.92 (m, 1H), 5.20 (s, 2H), 4.40 (bs, 2H), 3.33-3.28 (m, 2H), 2.99-2.94 (m, 2H), 1.37 (s, 9H).

### Preparation 23: Synthesis of tert-Butyl {2-[2-(2-hydrazinyl-2-oxoethyl)-2H-tetrazol-5-yl]ethyl}carbamate

The product tert-butyl {2-[2-(2-hydrazinyl-2-oxoethyl)-2H-tetrazol-5-yl]ethyl}carbamate was obtained by using same procedure described in Step-2 of Preparation-22 using non-polar product obtained from Step-1 product of Preparation-22.

Analysis:

Mass: 284.2(M−1), 286.2 (M+1); for Molecular weight: 285 and Molecular formula: C₁₀H₁₉N₇O₃.

¹HNMR (DMSO-d₆): δ 9.54 (bs, 1H), 6.93 (m, 1H), 5.29 (s, 2H), 4.40 (bs, 2H), 3.31-3.25 (m, 2H), 2.95-2.92 (m, 2H), 1.36 (s, 9H).

### Preparation 24: Synthesis of tert-butyl {3-[2-(2-hydrazinyl-2-oxoethyl)-2H-tetrazol-5-yl]propyl}carbamate

Synthesized by using tert-butyl [3-(1H-tetrazol-5-yl)propyl]carbamate (synthesized as per European Journal of Medicinal Chemistry (1984), 19(2), 181-6) and by following Steps 1-2 of Preparation-22.

Analysis:

Mass: 298 (M−1), 300 (M+1); for Molecular formula: C₁₁H₂₁N₇O₃, Molecular weight: 299.

¹HNMR: (DMSO-d₆): δ 9.57 (bs, 1H), 6.90-6.95 (m, 1H), 5.23 (s, 2H), 4.40 (bs, 2H), 3.02-2.97 (m, 2H), 2.82-2.78 (m, 2H), 1.81 (m, 2H), 1.37 (s, 9H).

### Preparation 25: Synthesis of tert-butyl {3-[1-(2-hydrazinyl-2-oxoethyl)-1H-tetrazol-5-yl]propyl}carbamate

Synthesized by using tert-butyl [4-(1H-tetrazol-5-yl)butyl[carbamate (synthesized according to the procedure described in European Journal of Medicinal Chemistry, 1984, 19(2), 181-6) as a starting material by following the procedures described for steps 1 and 2 of Preparation-22.

Analysis:

Mass: 298 (M−1), 300(M+1); for Molecular weight: 299 and Molecular formula: C₁₁H₂₁N₇O₃.

¹HNMR (DMSO-d₆): δ 9.57 (bs, 1H), 6.905 (m, 1H), 5.29 (s, 2H), 4.40 (bs, 2H), 3.02-2.97 (m, 2H), 2.82-2.78 (m, 2H), 1.81 (m, 2H), 1.37 (s, 9H).

### Preparation 26: Synthesis of tert-butyl {4-[2-(2-hydrazinyl-2-oxoethyl)-2H-tetrazol-5-yl]butyl}carbamate

Synthesized by using tert-butyl [4-(1H-tetrazol-5-yl)butyl]carbamate (synthesized according to the procedure described in European Journal of Medicinal Chemistry, 1984, 19(2), 181-6) as a starting material by following the procedures described for steps 1 and 2 of Preparation-22.

Analysis:

Mass: 312.3(M−1), 314.4(M+1); for Molecular weight: 313 and Molecular formula: C₁₂H₂₃N₇O₃.

¹HNMR (DMSO-d₆): δ 9.56 (bs, 1H), 6.81 (m, 1H), 5.07 (s, 2H), 3.31 (bs, 2H), 2.96-2.91 (m, 2H), 2.83-2.75 (m, 2H), 1.70 (m, 2H), 1.45 (m, 2H), 1.37 (s, 9H).

### Preparation 27: Synthesis of tert-butyl {4-[1-(2-hydrazinyl-2-oxoethyl)-1H-tetrazol-5-yl]butyl}carbamate

Synthesized by using tert-butyl [4-(1H-tetrazol-5-yl)butyl]carbamate (synthesized according to the procedure described in European Journal of Medicinal Chemistry, 1984, 19(2), 181-6) as a starting material by following the procedures described for Steps 1 and 2 of Preparation-22.

Analysis:

Mass: 312.3 (M−1), 314.4 (M+1); for Molecular weight: 313 and Molecular formula: C₁₂H₂₃N₇O₃.

¹HNMR (DMSO-d₆): δ 9.57 (bs, 1H), 6.81 (m, 1H), 5.29 (s, 2H), 4.40 (bs, 2H), 2.95-2.83 (m, 2H), 2.81-2.79 (m, 2H), 1.69 (m, 2H), 1.46(m, 2H), 1.36 (s, 9H).

### Preparation 28: Synthesis of (2S,5R)-6-benzyloxy-N′-2-{[({[(2S)-1-(tert-butoxycarbonyl)pyrrolidin-2-yl]carbonyl}oxy)methyl]-imidazole-1-yl}-acetyl -7-oxo-1,6-diazabicyclo[3.2.1]octane-2-carbohydrazide

Step 1: Synthesis of (2S,5R)-6-benzyloxy-N′-{[2-(tertiary-butyl-dimethyl-silanyloxymethyl)-1H-imidazol-1-yl]-1-oxoethyl}-7-oxo-1,6-diazabicyclo[3.2.1]octane-2-carbohydrazide: To a solution of sodium (2S,5R)-6-(benzyloxy)-7-oxo-1,6-diazabicyclo[3.2.1]octane-2-carboxylate (4.5 g, 15.8 mmol) in dimethylformamide (45 ml) was added EDC.HCl (4.52 g, 23.7 mmol) at 25-30° C. under stirring followed by 2-(tertiary-butyl-dimethyl-silanyloxymethyl)-1H-imidazole-1-carbohydrazide (4.71 g, 15.8 mmol, prepared according to the procedure described in preparation 36) and hydroxy benzotriazole monohydrate (2.41 g, 15.8 mmol). Finally, to the resulted reaction mass was added N-methyl morpholine (5.21 ml, 47.4 mmol) under stirring and completion of the reaction was monitored by TLC (chloroform:methanol, 9:1). After complete consumption of starting material the volatiles were distilled out on rotavapour till dryness. The reaction mass was partitioned in dichloromethane (45 ml) and water (45 ml). The aqueous layer was re-extracted with dichloromethane (2×45 ml). The organic extracts were combined and washed with brine (1×45 ml) and dried over anhydrous sodium sulphate. The volatiles were removed under reduced pressure, yielded crude, which was further purified by column chromatography (60-120 mesh size silica gel) using methanol: dichloromethane mixture as an eluent, yielded 5.5 g of (2S,5R)-6-benzyloxy-N′-{[2-(tertiary-butyl-dimethyl-silanyloxymethyl)-1H-imidazol-1-yl]-1-oxoethyl}-7-oxo-1,6-diazabicyclo[3.2.1]octane-2-carbohydrazide in 63% yield.

Step 2: Synthesis of (2S,5R)-6-benzyloxy-N′-{[2-(hydroxymethyl)-1H-imidazol-1-yl]-1-oxoethyl}-7-oxo-1,6-diazabicyclo[3.2.1]octane-2-carbohydrazide: To a solution of (2S,5R)-6-benzyloxy-N′-{[2-(tertiary-butyl-dimethyl-silanyloxymethyl)-1H-imidazol-1-yl]-1-oxoethyl}-7-oxo-1,6-diazabicyclo[3.2.1]octane-2-carbohydrazide (3.3 g, 6.08 mmol) in tetrahydrofuran (66 ml) was added tetrabutylammonium fluoride 1M solution in tetrahydrofuran (18.3 ml, 18.2 mmol) at 0° C. under stirring. The reaction was stirred at ambient temperature by confirming the progress of reaction by performing TLC (chloroform:methanol, 9:1). After complete consumption of starting material the reaction mixture was distilled out on rotavapour till dryness. The reaction mass was partitioned in dichloromethane (33 ml) and water (33 ml). The aqueous layer was re-extracted with dichloromethane (2×33 ml) dried over anhydrous sodium sulphate. The volatiles were removed under reduced pressure. Crude was purified compound by column chromatography (60-120 mesh size silica gel) using methanol: dichloromethane as an eluent yielded 2.2 g of (2S,5R)-6-benzyloxy-N′-{[2-(hydroxymethyl)-1H-imidazol-1-yl]-1-oxoethyl}-7-oxo-1,6-diazabicyclo[3.2.1]octane-2-carbohydrazide in 85% yield.

Step 3: Synthesis of (2S,5R)-6-benzyloxy-N′-2-{[({[(2S)-1-(tert-butoxycarbonyl)pyrrolidin-2-yl]carbonyl]oxy)methyl}-imidazole-1-yl}-acetyl-7-oxo-1,6-diazabicyclo[3.2.1]octane-2-carbohydrazide: To a solution of (2S)-1-(tert-butoxycarbonyl)pyrrolidine-2-carboxylic acid (0.502 g, 2.33 mmol) in dichloromethane (15 ml) was added EDC.HCl under stirring followed by (2S,5R)-6-benzyloxy-N′-{[2-(hydroxymethyl)-1H-imidazol-1-yl]carbonyl}-7-oxo-1,6-diazabicyclo[3.2.1]octane-2-carbohydrazide (1.0 g, 2.33 mmol) and 4-dimethylamino pyridine (0.426 g, 3.49 mmol) at 25-30° C. The reaction mass was stirred at ambient temperature and progress of the reaction was checked by thin layer chromatography (chloroform: methanol, 9:1), after complete consumption of starting material was added water (15 ml) to the reaction mixture. The layers were separated and organic layer was dried over anhydrous sodium sulfate. The organic layer concentrated under vacuum till dryness, to yield 0.9 g of (2S,5R)-6-benzyloxy-N′-2-{[({[(2S)-1-(tert-butoxycarbonyl)pyrrolidin-2-yl]carbonyl}oxy)methyl]-imidazole-1-yl}-acetyl -7-oxo-1,6-diazabicyclo[3.2.1]octane-2-carbohydrazide as a brown oil in 62% yield.

Analysis:

Mass: 624.5(M−1), 626.5 (M+1); for Molecular weight: 625.69 and Molecular formula: C₃₀H3₉N₇O₈.

### Preparation 29: Synthesis of (2S,5R)-6-benzyloxy-N′-[{2-[({(2S)-2-[(tert-butoxycarbonyl)amino]propanoyl}oxy)methyl]-imidazol-1-yl}-acetyl]-7-oxo-1,6-diazabicyclo[3.2.1]octane-2-carbohydrazide

To a solution of (2S)-2-[(tert-butoxycarbonyl)amino]propanoic acid (0.794 g, 4.2 mmol) in dichloromethane (27 ml) was added EDC.HCl (1.2 g, 6.3 mmol) at 25-30° C. under stirring. After 30 minutes of stirring at the same temperature added (2S,5R)-6-benzyloxy-N′-{[2-(hydroxymethyl)-1H-imidazol-1-yl]acetyl}-7-oxo-1,6-diazabicyclo[3.2.1]octane-2-carbohydrazide (1.8 g, 4.2 mmol, prepared according to the procedure described for step 2 product of preparation 28) followed by 4-dimethylamino pyridine (0.77 g, 6.3 mmol). After completion of the reaction as monitored by TLC (chloroform: methanol, 9:1) was added water (27 ml) to the reaction mixture; and the layer were separated. The collected organic layer was dried over anhydrous sodium sulfate and concentrated under reduced pressure till dryness to yield 1.2 g of (2S,5R)-6-benzyloxy-N′-[{2-[({(2S)-2-[(tert-butoxycarbonyl)amino]propanoyl}oxy)methyl]-imidazol-1-yl}-acetyl]-7-oxo-1,6-diazabicyclo[3.2.1]octane-2-carbohydrazide as off white solid in 48% yield.

Analysis:

Mass: 598.5 (M−1), 600.5 (M+1); for Molecular weight: 599.65 and Molecular formula: C₂₈H₃₇N₇O₈.

### Preparation 30: Synthesis of tert-Butyl [1-(2-hydrazinyl-2-oxoethyl)-1H-imidazol-4-yl]carbamate

To a solution ethyl {4-[(tert-butoxycarbonyl) amino]-1H-imidazol-1-yl}acetate (3.6 g, 13 mmol, prepared according to the procedure described in PCT 2014182829) in ethanol (36 ml) was added hydrazine hydrate (1.07 g, 26 mmol) at 25-30° C. The progress of reaction was monitored by TLC (chloroform: methanol, 9:1). After complete consumption of starting material the precipitates obtained were filtered on Buchner funnel and washed with ethanol (2×10 mL). The product was dried under reduced pressure till constant weight to get 3.2 g of tert-butyl [1-(2-hydrazinyl-2-oxoethyl)-1H-imidazol-4-yl]carbamate as white solid in 94% yield.

### Preparation 31: Synthesis (2S,5R)-N′-[(4-tert-butoxycarbonylamino-2-oxopyrimidin-1(2H)-yl)acetyl]-6-benzyloxy-7-oxo-1,6-diazabicyclo[3.2.1]octane-2-carbohydrazide

To a solution of {4-[(tert-butoxycarbonyl)amino]-2-oxopyrimidin-1(2H)-yl}acetic acid (1.5 g, 5 mmoles, prepared according to the procedure described in European Journal of Organic Chemistry, (18), 3141-3148, 2008) in dimethylformamide (20 mL) was added N-methylmorpholine (3.2 ml, 22 mmol), EDC.HCl (21. g,11 mmol) and finally TFA salt of (2S, 5R)-6-(benzyloxy)-7-oxo-1,6-diazabicyclo[3.2.1]octane-2-carbohydrazide (3.3 g, 8 mmoles, prepared as per the reference WO2013030733) and HOBt (0.75 g, 5 mmol). The progress of reaction was monitored by TLC (Chloroform: MeOH, 9:1). After complete consumption of starting material the reaction mixture poured onto water under stirring, the precipitates thus obtained were filtered on Buchner funnel. The crude product was purified by column chromatography using chloroform: methanol as an eluent. The product containing combined fractions were distilled out and dried under reduced pressure till constant weight to provide 1.2 g of (2S,5R)-N′-[(4-tert-butoxycarbonylamino-2-oxopyrimidin-1(2H)-yl)acetyl]-6-benzyloxy-7-oxo-1,6-diazabicyclo[3.2.1]octane-2-carbohydrazide as white solid in 34.5% yield.

Analysis:

Mass: 540.4 (M−1), 542.4 (M+1); for Molecular weight: 541.5 and Molecular formula: C₂₅H₃₁N₇O₇.

¹H NMR (CDCl₃): δ 10.29 (s, 1H), 10.19 (s, 1H), 10.00 (s, 1H), 7.95-7.97 (d, 1H), 7.37-7.44 (m, 5H), 6.97 (s, 1H), 4.93 (s, 2H), 4.59 (s, 2H), 3.83-3.84 (d, 1H), 3.68 (s, 1H), 3.19-3.22 (d, 1H), 2.87-2.96 (d, 1H), 1.62-2.01(m, 4H), 1.49 (s, 9H).

Purity by HPLC: 86.95%.

### Preparation 32: Synthesis of 2{5-[2-(tert-butyl)-dimethyl)-silanyloxyl)-ethyl]-2H-tetrazol-2-yl}-acetohydrazide

Step 1: Synthesis of 3-(tert-butyl-dimethyl-silanyloxy)-propionitrile: To a solution of 3-hydroxy-propionitrile (50 g, 704.2 mmol) in dimethylformamide (250 ml) was added imidazole (72 g, 1056 mmol) under stirring at 25-30° C. and allowed to cool to 0° C. To the resulted reaction mass was added solid tert-butyl dimethylsilyl chloride (106 g, 704 mmol) under stirring. The progress of reaction was monitored by TLC (hexane: ethyl acetate, 1:1). After complete consumption of starting material the reaction was quenched by adding it to water (2.5 L) and extracted with diethyl ether (3×500 ml). The combined organic extract was dried over anhydrous sodium sulfate and concentrated under reduced pressure to yield 130 g of 3-(tert-butyl-dimethyl-silanyloxy)-propionitrile as colourless oil in 100% yield.

Step 2: Synthesis of 5-(2-(tert-butyl-dimethyl-silanyloxyl)-ethyl-1H-tetrazole: To a stirred solution of 3-(tert-butyl-dimethyl-silanyloxy)-propionitrile (130 g, 702 mmol, obtained from step 1) in dimethylformamide (650 ml) was added triethyl amine HCl (289 g, 2108 mmol) and sodium azide (137 g, 2108 mmol) under stirring. The reaction mixture was heated at 100-110° C. under stirring till completion. The progress of reaction was monitored by TLC (chloroform: methanol, 9:1). After complete consumption of starting material the reaction mixture was cooled to ambient temperature and quenched by adding it to water (6.5 L) and extracted with ethyl acetate (2×1 L). The combined organic extract was dried over anhydrous sodium sulphate and on concentration yielded 110 g of 5-(2-(tert-butyl-dimethyl-silanyloxyl)-ethyl-1H-tetrazole as pale yellow liquid in 69% yield.

Step 3: Synthesis of {5-[2-(tert-butyl-dimethyl-silanyloxy)-ethyl]-tetrazol-1-yl}-acetic acid ethyl ester and {5-[2-(tert-butyl-dimethyl-silanyloxy)-ethyl]-tetrazol-2-yl}-acetic acid ethyl ester. To a stirred solution of 5-(2-(tert-butyl-dimethyl-silanyloxyl)-ethyl-1H-tetrazole (100 g, 438 mmol, obtained from step 2) in dimethylformamide (500 ml) was added potassium carbonate (9 g, 657 mmol) under stirring at 25-30° C. After 10 minutes of stirring was added ethyl bromoacetate (48 ml, 438 mmol) using addition funnel at 10-15° C. The progress of reaction was monitored by TLC (hexane: ethyl acetate, 1:1). After complete consumption of starting material the insoluble inorganic was removed by filtration on celite bed and washed with ethyl acetate (2×200 ml). The filtrate was poured on to water (5L) under stirring and extracted with ethyl acetate (2×500 ml). The organic extract was dried over anhydrous sodium sulfate and on concentration yielded isomers mixture. The isomers were separated by column chromatography (silica gel 100:200 mesh size) using Ethyl acetate: Hexane as an eluent. The non-polar isomer {5-[2-(tert-butyl-dimethyl-silanyloxy)-ethyl]-2H-tetrazol-2-yl}-acetic acid ethyl ester was obtained by using 10% ethyl acetate in hexane in 40 g yield and the polar isomer {5-[2-(tert-butyl-dimethyl-silanyloxy)-ethyl]tetrazol-1-yl}-acetic acid ethyl ester was obtained at 15-20% ethyl acetate in hexane in 53 g yield.

Step 4: Synthesis of 2{5-[2-(tert-butyl)-dimethyl)-silanyloxyl)-ethyl]-2H-tetrazol-2-yl}-acetohydrazide: To a solution of {5-[2-(tert-butyl-dimethyl-silanyloxy)-ethyl]-2H-tetrazol-2-yl}-acetic acid ethyl ester (30 g, 95.5 mmol, non-polar product from Step-3) in ethanol (300 ml) was added hydrazine hydrate (4.8 ml, 95.5 mmol) under stirring at 25-30° C. The progress of reaction was monitored by TLC (chloroform: methanol, 9:1). After complete consumption of starting material, the volatiles were removed under reduced pressure to provide 22 g of 2{5-[2-(tert-butyl)-dimethyl)-silanyloxyl)-ethyl]-2H-tetrazol-2-yl}acetohydrazide as colorless liquid in 77% yield.

Analysis:

Mass: 299.2 (M−1), 301.2 (M+1); for Molecular weight: 300 and Molecular formula: C₁₁H₂₄N₆O₂Si.

¹HNMR (DMSO-d₆): δ 9.57 (bs, 1H), 5.10 (s, 2H), 4.38 (bs, 2H), 3.96-3.91 (m, 2H), 3.00-2.97 (m, 2H), 0.86 (s, 9H), 0.00 (s, 6H).

### Preparation 33: Synthesis of 2{5-[2-(tert-butyl)-dimethyl)-silanyloxyl)-ethyl]-1H-tetrazol-1-yl}-acetohydrazide

Similarly using {5-[2-(tert-butyl-dimethyl-silanyloxy)-ethyl]-tetrazol-1-yl}-acetic acid ethyl ester (polar product from step 3 of preparation 32) by following the procedure described in Step-4 of Preparation-32 yielded 2{5-[2-(tert-butyl)-dimethyl)-silanyloxyl)-ethyl]-1H-tetrazol-1-yl}-acetohydrazide.

Analysis:

Mass: 299.2 (M−1), 301.2 (M+1); for Molecular weight: 300 and Molecular formula: C₁₁H₂₄N₆O₂Si.

¹HNMR (DMSO-d₆): δ 9.57 (bs, 1H), 5.10 (s, 2H), 4.03 (bs, 2H), 3.96-3.91 (m, 2H), 3.00 (m, 2H), 0.81 (s, 9H), 0.00 (s, 6H).

### Preparation 34: Synthesis of {2-[2-(tert-butyl-dimethyl-silanyloxy)-ethyl]-imidazol-1-yl}acetic acid hydrazide

Step 1: Synthesis of 2-[2-(tert-butyl-dimethyl-silanyloxy)-ethyl]-1H-imidazole: To a solution of 2-(1H-imidazol-2-yl)ethanol (5 g, 44.6 mmol, prepared according to the procedure described in Synthesis, 12, 325-528; 2002) in dimethylformamide (50 ml) was added imidazole (4.55 g, 66.9 mmol) under stirring at 25-30° C. followed by the addition of tert-butyldimethylsilyl chloride (7 g, 44.6 mmol) under continuous stirring. The progress of reaction was monitored by mass spectrometry. After complete consumption of starting material the reaction mixture was poured on to water (500 ml) and extracted with ethyl acetate (2×250 ml). The combined organic extract was dried over anhydrous sodium sulfate and concentrated to yield 8 g of 2-[2-(tert-butyl-dimethyl-silanyloxy)-ethyl]-1H-imidazole as pale yellow liquid in 80% yield.

Step 2: Synthesis of {2-[2-(tert-Butyl-dimethyl-silanyloxy)-ethyl]-imidazol-1-yl}-acetic acid ethyl ester: To a solution of 2-[2-(tert-butyl-dimethyl-silanyloxy)-ethyl]-1H-imidazole (7.5 g, 33 mmol, obtained from step 1) in dimethylformamide (40 ml) was added potassium carbonate (4.57 g, 33 mmol) under stirring at 25-30° C. After 10-15 minutes of stirring the reaction mass was cooled to 10-15° C. and at this temperature was added ethyl bromoacetate (5.54 g, 33 mmol) by using addition funnel under continuous stirring. The progress of reaction was monitored by TLC (chloroform:methanol, 9:1). After complete consumption of starting material the reaction mixture was filtered through celite bed to remove solid material. The celite bed was washed with ethyl acetate (2×25 ml). The filtrate was partitioned between water (400 ml) and ethyl acetate (200 ml). The aqueous reaction mixture was re-extracted with ethyl acetate (200 ml). The combined organic extract was dried over anhydrous sodium sulfate and concentrated, to provide 7.2 g of thick oil compound of {2-[2-(tert-butyl-dimethyl-silanyloxy)-ethyl]-imidazol-1-yl}-acetic acid ethyl ester in 69% yield.

Step 3: Synthesis of {2-[2-(tert-butyl-dimethyl-silanyloxy)-ethyl]-imidazol-1-yl}-acetic acid hydrazide: To a solution of {2-[2-(tert-butyl-dimethyl-silanyloxy)-ethyl]-imidazol-1-yl}-acetic acid ethyl ester (7 g, 22.4 mmol) in ethanol (70 ml) at 25-30° C. was added hydrazine hydrate (1.12 ml, 22.4 mmol) under stirring. The progress of reaction was monitored by TLC (Chloroform Methanol 9:1). After complete consumption of starting material the volatiles were removed under reduced pressure to yield 6.5 g of {2-[2-(tert-butyl-dimethyl-silanyloxy)-ethyl]-imidazol-1-yl}-acetic acid hydrazide as pale yellow liquid in 97% yield.

Analysis:

Mass: 297.3 (M−1), 299.3 (M+1); for Molecular weight: 298 and Molecular formula: C₁₃H₂₆N₄O₂Si.

¹HNMR (DMSO-d₆): δ 9.32 (bs, 1H), 6.95 (s, 1H), 6.71 (s, 1H), 4.51 (bs, 2H), 4.32 (bs, 2H), 3.85-3.82 (m, 2H), 2.76-2.78 (m, 2H), 0.81 (s, 9H), 0.02 (s, 6H).

### Preparation 35: Synthesis of a mixture of 2-[4-(tertiary-butyl-dimethyl-silanyloxy methyl)-1H-1,2,3-triazol-1-yl]acetohydrazide and 2-[5-(tertiary-butyl-dimethyl-silanyloxy methyl)-1H-1,2,3-triazol-1-yl]acetohydrazide

Step 1: Synthesis and separation of benzyl [4-(tertiary-butyl-dimethyl-silanyloxy methyl)-2H-1,2,3-triazol-2-yl]acetate, benzyl [5-(tertiary-butyl-dimethyl-silanyloxy methyl)-1H-1,2,3-triazol-1-yl]acetate and benzyl [4-(tertiary-butyl-dimethyl-silanyloxy methyl)-1H-1,2,3-triazol-1-yl]acetate: To a solution of 4-tertiary-butyl-dimethyl-silanyloxy methyl-2H-1,2,3-triazol (26 g, 122 mmol, prepared according to the procedure described in US20150266867) in dimethylformamide (130 ml) was added potassium carbonate (18.5 g, 134 mmol) under stirring followed by the drop-wise addition of benzyl bromoacetate (20 ml, 122 mmol) at 25-30° C. The progress of reaction was monitored by TLC (hexane: ethyl acetate, 8:2). After complete consumption of starting material the reaction mixture was filtered through celite bed to remove insolubles and filtrate was concentrated, to the resulted concentrated mass was added to water (250 mL). The aqueous reaction mixture was extracted with ethyl acetate (2×250 ml). The organic extracts were combined and washed with brine (125 mL), dried over anhydrous sodium sulfate. The volatiles were removed under reduced pressure to yield a mixture of benzyl [4-(tertiary-butyl-dimethyl-silanyloxy methyl)-2H-1,2,3-triazol-2-yl]acetate, benzyl [5-(tertiary-butyl-dimethyl-silanyloxy methyl)-1H-1,2,3-triazol-1-yl]acetate and benzyl [4-(tertiary-butyl-dimethyl-silanyloxy methyl)-1H-1,2,3-triazol-1-yl]acetate (37 g), these isomers were separated by column chromatography (100-200 mesh size silica gel) using hexane: ethyl acetate (8:2) as an eluent. The non-polar isomer benzyl [4-(tertiary-butyl-dimethyl-silanyloxy methyl)-2H-1,2,3-triazol-2-yl]acetate was eluted at 15-20% ethylacetate in hexane, to provide 13 g of pure product. The mixture of polar isomers benzyl [5-(tertiary-butyl-dimethyl-silanyloxymethyl)-1H-1,2,3-triazol-1-yl]acetate and benzyl [4-(tertiary-butyl-dimethyl-silanyloxy methyl)-1H-1,2,3-triazol-1-yl]acetate was eluted at 40-45% ethyl acetate in hexane, to provide 15 g of product.

Step 2: To a solution of benzyl [5-(tertiary-butyl-dimethyl-silanyloxy methyl)-1H-1,2,3-triazol-1-yl]acetate and benzyl [4-(tertiary-butyl-dimethyl-silanyloxy methyl)-1H-1,2,3-triazol-1-yl]acetate (2 g, 5.5 mmol, polar isomers mixture from step 1) in ethanol (10 ml) was added hydrazine hydrate monohydrate (0.415 mL, 8.3 mmol) at 25-30° C. under stirring. The progress of reaction was monitored by TLC (chloroform: methanol, 9:1). After complete consumption of starting material the reaction mixture was distilled out completely to obtain oily mass containing a mixture of 2-[4-(tertiary-butyl-dimethyl-silanyloxy methyl)-1H-1,2,3-triazol-1-yl]acetohydrazide and 2-[5-(tertiary-butyl-dimethyl-silanyloxy methyl)-1H-1,2,3-triazol-1-yl]acetohydrazide, 2 g yield, used as such in the next step.

Analysis:

Mass: 284.3 (M−1), 286.3(m+1); for Molecular weight: 285 and Molecular formula: C₁₁H₂₃N₅O₂Si.

### Preparation 36: Synthesis of 2-[2-(tertiary-butyl-dimethylsilanyloxy-methyl)-1H-imidazol-1-yl]acetohydrazide

Step 1: Synthesis of 1-benzyl-[2-(tertiary-butyl-dimethyl-silanyloxy)methyl]-1H-imidazole: To a solution of (1-benzyl-1H-imidazol-2-yl)methanol (8 g, 42.5 mmol, prepared according to the procedure described in PCT. Int. application 2011071057) in dimethylformamide (80 ml) was added imidazole (5.78 g, 85 mmol) under stirring at 25-30° C. followed by tert-butyldimethylsilyl chloride (7.68 g, 51 mmol). The progress of reaction was monitored by TLC (chloroform: methanol, 9:1). After complete consumption of starting material the reaction mixture was added to water (800 ml) and extracted with ethyl acetate (2×400 ml). The organic extracts were dried over sodium sulfate and concentrated. The crude compound was purified by column chromatography (silica gel 60:120 mesh size) using chloroform: methanol as an eluent to yield 6.1 g of 1-benzyl-[2-(tertiary-butyl-dimethyl-silanyloxy)methyl]-1H-imidazole as pale yellow liquid in 47.5% yield.

Step 2: Synthesis of 2-[(tert-butyl-dimethyl-silanyloxy)-methyl]-1H-imidazole: To a solution of 1-benzyl-[2-(tertiary-butyl-dimethyl-silanyloxy)methyl]-1 H-imidazole (14 g, 46.3 mmol, step 1 product) in methanol (140 ml) was added Pd(OH)₂ (3.5 g, 10% on carbon) and hydrogenated at 50 psi. The progress of reaction was monitored by TLC (chloroform: methanol, 9:1). On complete consumption of starting material the reaction mixture was filtered on celite bed and washed with methanol (2×28 ml). The filtrate was concentrated under reduced pressure to get 6.3 g of desired product 2-[(tert-butyl-dimethyl-silanyloxy)-methyl]-1H-imidazole in 64% yield.

Step 3: Synthesis of ethyl [2-(2-{[tert-butyl(dimethyl)-silanyl]oxy}methyl)-1H-imidazol-1-yl]acetate: To a solution of 2-[(tert-butyl-dimethyl-silanyloxy)-methyl]-1H-imidazole (6.3 g, 29.6 mmol, step 2 product) in dimethylformamide (32 ml) was added potassium carbonate (4.92 g, 35.5 mmol) under stirring at 25-30° C. followed by drop wise addition of ethyl bromoacetate (4.93 g, 29.6 mmol) at 10-15° C. The progress of reaction was monitored by TLC (chloroform: methanol, 9:1). After complete consumption of starting material the reaction mixture was filtered through celite bed to remove insoluble inorganic material. The celite bed was washed with ethyl acetate (2×25 ml). The collected filtrate was distilled under vacuum and purified by column chromatography (60-120 mesh size silica gel) using methanol: dichloromethane mixture as an eluent to yield 6.2 g of ethyl [2-(2-{[tert-butyl(dimethyl)-silanyl]oxy}methyl)-1H-imidazol-1-yl]acetate in 70% yield.

Step 4: Synthesis of 2-[2-(tertiary-butyl-dimethylsilanyloxy-methyl)-1H-imidazol-1-yl]acetohydrazide: To a stirred solution of ethyl [2-(2-{[tert-butyl(dimethyl)-silanyl]oxy}methyl)-1H-imidazol-1-yl]acetate (6 g, 20.13 mmol, step 3 product) in ethanol (60 ml) at 25-30° C. was added hydrazine hydrate (1.46 ml, 30.2 mmol) under stirring. The progress of reaction was monitored by TLC (chloroform: methanol, 9:1). After complete consumption of starting material the volatiles were removed under reduced pressure to yield 4.54 g of 2-[2-(tertiary-butyl-dimethylsilanyloxy-methyl)-1H-imidazol-1-yl]acetohydrazide as off white solid in 80% yield.

Analysis:

Mass: 283.4 (M−1), 285.4 (M+1); for Molecular weight: 284.44 and Molecular formula: C₁₂H₂₄N₄O₂Si.

### Preparation 37: Synthesis of lithium salt of [4-(tertiary-butyl-dimethyl-silanyloxy methyl)-2H-1,2,3-triazol-2-yl]acetic acid

A solution of lithium hydroxide (0.511 g, 12.1 mmol) in water (20 ml) was slowly added to a pre-cooled solution of benzyl [4-tertiary-butyl-dimethyl-silanyloxy methyl)-2H-1,2,3-triazol-2-yl]acetate (4 g, 11.0 mmol, the non-polar product from step 1 of preparation 35) in THF (20 ml) and continued stirring at 0° C. The progress of reaction was monitored by TLC (hexane: ethyl acetate 5:5). On complete consumption of starting material the reaction mixture was concentrated under reduced pressure ensured to complete removal of THF. The resulted aqueous reaction mixture was extracted with ethyl acetate (2×25 mL), pH of reaction mass was adjusted to 5 by using 5% KHSO₄ solution and concentrated under reduced pressure to get 3.5 g of lithium salt of [4-(tertiary-butyl-dimethyl-silanyloxy methyl)-2H-1,2,3-triazol-2-yl[acetic acid.

Analysis:

Mass: 270.2 (M−1), 272.3 (M+1); for Molecular weight: 276 and Molecular formula: C₁₁H₂₀N₃O₃SiLi.

¹HNMR (DMSO-d₆): δ 7.51 (s, 1H), 4.69 (s, 2H), 4.63 (s, 2H), 0.880 (s, 9H), 0.074 (s, 6H).

### Preparation 38: Synthesis of (2S,5R)-N-{2-[5-(2-aminoethyl)-2H-tetrazol-2-yl]ethoxy}-7-oxo-6-benzyloxy-1,6-diazabicyclo[3.2.1]octane-2-carboxamide

Step-1: Synthesis of a mixture of 2-{2-[5-(2-tertiary butoxy carbonyl aminoethyl)-2H-tetrazol-2-yl]ethoxyl-1H-isoindole-1,3(2H)-dione and 2-{2-[5-(2-tertiary butoxy carbonyl aminoethyl)-1H-tetrazol-1-yl]ethoxy}-1H-isoindole-1,3(2H)-dione: To a solution of 2-(2-bromoethoxy) isoindoline-1,3-dione (10 g, 37 mmol) and tert-butyl 2-(1H-tetrazol-5-yl)ethylcarbamate (7.88 g, 37 mmol) in dimethyl formamide (50 ml) was added cesium carbonate ((14.48 g, 44 mmole) in portion wise at 25-30° C. under stirring. The reaction mixture was stirred for further 16 hours and filtered; the filtrate was slowly poured into chilled water (400 ml) under stirring and continued stirring for 30 minutes. The formed precipitates of compound were filtered and washed with water (40 ml). The solid compound was dried at 40° C. for 2 hour under high vacuum, yielded 12 g. The compound was column purified using hexane and acetone as an eluent. The mixture was isolated at 10-15% concentration of acetone in hexane to yield 10 g of 2-{2-[5-(2-tertiary butoxy carbonyl aminoethyl)-2H-tetrazol-2-yl]ethoxy}-1H-isoindole-1,3(2H)-dione and 2-{2-[5-(2-tertiary butoxy carbonyl amino ethyl)-1H-tetrazol-1-yl]ethoxy}-1H-isoindole-1,3(2H)-dione in 67% yield.

Analysis:

Mass: 403 (M+1); for Molecular weight: 402 and Molecular formula: C₁₈H₂₂N₆O₅.

Step-2: Synthesis of mixture of tert-butyl (2-{1-[2-(aminooxy)ethyl]-1H-tetrazol-5-yl}ethyl)carbamate and tert-butyl (2-{2-[2-(aminooxy)ethyl]-2H-tetrazol-5-yl}ethyl)carbamate: Hydrazine hydrate monohydrate (1.86 ml, 037.3 mmol) was added to a solution of 2-{2-[5-(2-tertiary-butoxy-carbonyl-amino ethyl)-2H-tetrazol-2-yl]ethoxy}-1H-isoindole-1,3 (2H)-dione and 2-{2-[5-(2-tertiary-butoxy-carbonyl-amino ethyl)-1H-tetrazol-1-yl]ethoxy}-1H-isoindole-1,3(2H)-dione (10 g, 24.8 mmol, obtained from step 1) in dichloromethane (10 ml) under stirring at room temperature. After completion of 2 hours of stirring at same temperature the TLC confirms the completion of reaction. The reaction mass was filtered and the solid was washed with dichloromethane (50 ml). The collected filtrate was concentrated at 40° C. under vacuum to yield 6.75 g of the product, which was used further without any purification.

Analysis:

Mass: 273 (M+1); for Molecular weight: 272 and Molecular formula: C₁₀H₂₀N₆O₃.

Step 3: Synthesis of (2S,5R)-N-{2-[5-(2-tertiary butoxy carbonyl aminoethyl)-2H-tetrazol-2-yl]ethoxy}-6-benzyloxy-7-oxo-1,6-diazabicyclo[3.2.1]octane-2-carboxamide and (2S,5R)-N-{2-[5-(2-tertiary butoxy carbonyl aminoethyl)-1H-tetrazol-1-yl]ethoxyl-6-benzyloxy-7-oxo-1,6-diazabicyclo[3.2.1]octane-2-carboxamide: To a solution of (2S,5R)-6-(benzyloxy)-7-oxo-1,6-diaza-bicyclo[3.2.1]octane-2-carboxylic acid (6.2 g, 22 mmol) in dimethylformamide (50 ml) was added EDC.HCl (6.45 g, 33.6 mmol) at ambient temperature under stirring. To this was added N-methyl morpholine (7.23 ml, 67.3 mmol) followed by HOBT (3.04 g, 22 mmol) under stirring and reaction mixture was stirred for 5 minutes and cooled to 15° C. and a solution of mixture of tert-butyl (2-{1-[2-(aminooxy)ethyl]-1H-tetrazol-5-yl}ethyl)carbamate and tert-butyl (2-{2-[2-(aminooxy)ethyl]-2H-tetrazol-5-yl}ethyl)carbamate (6.72 g, 24.7 mmol) in dimethylformamide (12 ml) was slowly added and allowed to reach ambient temperature and stirred for 16 hours. The progress of reaction was monitored by TLC, after complete conversion of starting material the reaction mixture was slowly poured onto chilled water (500 ml) and extracted with ethyl acetate (2×150 ml). The combined organic layer was washed with brine (75 ml) and organic layer was dried over anhydrous sodium sulfate. The organic layer was concentrated on rotavapour to provide 4.2 g of crude compound. The crude compound was column purified by using hexane and acetone as an eluent. The non-polar spot isolated at 10-15% concentration of acetone in hexane yielded 1.6 g of (2S,5R)-N-{2-[5-(2-tertiary butoxy carbonyl aminoethyl)-2H-tetrazol-2-yl]ethoxy}-6-benzyloxy-7-oxo-1,6-diazabicyclo[3.2.1]octane-2-carboxamide. The polar spot isolated at 20-25% concentration of acetone in hexane and the pure fractions were collected and concentrated on Rota evaporator to yield 1.4 g of (2S,5R)-N-{2-[5-(2-tertiary butoxy carbonylaminoethyl)-1H-tetrazol-1-yl]ethoxy}-6-benzyloxy-7-oxo-1,6-diazabicyclo[3.2.1]octane-2-carboxamide as white compound, in 25% yield.

Analysis:

Mass: 531 (M+1); for Molecular weight: 530 and Molecular formula: C₂₄H₃₄N₈O₆.

¹H NMR (DMSO-d₆): δ 9.30 (s, 1H), 7.43-7.33 (m, 5H), 5.05-5.02 (m, 1H), 4.90-4.83 (m, 3H), 4.47-4.43 (m, 2H), 3.92-3.91 (d, 1H, J=7.2 Hz), 3.59-3.57 (m, 2H), 3.30 (s, 1H), 3.11-3.08 (m, 2H), 2.99-2.97(m, 1H), 2.71-2.68 (m, 1H), 2.29-2.24 (m, 2H),m 2.04-1.89 (m, 3H), 1.49 (s, 9H).

### Preparation 39: Synthesis of 1-[2-(aminooxy)ethyl]-1H-1,2,4-triazole

Step 1: Synthesis of 2-(2-bromoethoxy)-1H-isoindole-1,3(2H)-dione: To a solution of N-hydroxy phthalimide (160 g, 98.1 mmol) in dimethylformamide (1150 ml) was slowly added triethylamine (344 ml, 245.2 mmol) under stirring. To this reaction mixture was added dibromoethane (127 ml, 147 mmol) in dimethylformamide (127 ml) at room temperature over 30 min. After 16 hours of stirring at the same temperature the completion of the reaction was confirmed by performing the thin layer chromatography. Then the resulted reaction mixture was filtered to remove inorganic salts and washed with dimethylformamide (127 ml). The filtrate was slowly poured into chilled water (8.5 1) and the mixture was stirred for 30 minutes. The formed precipitates were filtered, washed with water (200 ml) and dried at 40° C. for 3 hours under high vacuum to provide 55 g of 2-(2-bromoethoxy)-1H-isoindole-1,3(2H)-dione as a white solid in 21% yield.

Step 2: Synthesis of 2-[2-(1H-1,2,4-triazol-1-yl)ethoxy]-1H-isoindole-1,3(2H)-dione: To a solution of 2-(2-bromoethoxy)isoindoline-1,3-dione (20 g, 7 mmol, step 1 product) in dimethylformamide (100 ml) was added 1H-1,2,4 triazole (5.2 g, 7.52 mmol) and cesium carbonate (26.5 g, 8.13 mmol) under stirring at 25-30° C. After 16 hours of stirring, the reaction mixture was filtered through filter paper and the filtrate was slowly poured into chilled water (700 ml) under stirring. After 30 minutes, the separated precipitates were filtered and washed with water (50 ml). The filtered precipitates were dried at 40° C. for 2 hour under high vacuum to provide 10.5 g of 2-[2-(1H-1,2,4-triazol-1-yl)ethoxy]-1H-isoindole-1,3 (2H)-dione as white solid in 55% yield.

Analysis:

Mass: 259.2 (M+1) for Molecular weight: 258 and for Molecular formula: C₁₂H₁₀N₄O₃.

Step 3: Synthesis of 1-[2-(aminooxy)ethyl]-1H-1,2,4-triazole: To a solution of 2-[2-(1H-1,2,4-triazol-1-yl)ethoxy]-1H-isoindole-1,3 (2H)-dione (10 g, 3.87 mmol) in dichloromethane (150 ml) was added hydrazine hydrate (2.8 ml, 5.73 mmol) under stirring at room temperature. After 1 hour, the progress of reaction was monitored by thin layer chromatography. After completion of the reaction, the insoluble solid was filtered out and washed with dichloromethane (20 ml). The filtrate was concentrated at 40° C. under high vacuum and to the concentrated mass ethyl acetate (35 ml) was added under stirring. The separated insoluble solid was filtered out and washed with ethyl acetate (10 ml). The filtrate was concentrated at 40° C. under high vacuum to obtain 5.5 g of 1-[2-(aminooxy)ethyl]-1H-1,2,4-triazole in about 100% yield, this was used in the next step without further purification.

### Preparation 40: Synthesis of mixture of ethyl 1-[-2-(aminooxy)ethyl]-1H-tetrazole-5-carboxylate and ethyl 2-[2-(aminooxy)ethyl]-2H-tetrazole-5-carboxylate

Step 1: Synthesis of mixture of ethyl 1-{2-[(1,3-dioxo-1,3-dihydro-2H-isoindol-2-yl)oxy]ethyl}-1H-tetrazole-5-carboxylate and ethyl 2-{2-[(1,3-dioxo-1,3-dihydro-2H-isoindol-2-yl)oxy]ethyl}-2H-tetrazole-5-carboxylate: To a mixture of 2-(2-bromoethoxy)isoindoline-1,3-dione (20 g, 74 mmol, prepared according to the procedure described in step 1 of preparation 10) and 2H-tetrazole-5-carboxylic acid ethyl ester (10.51 g, 74 mmol) in dimethylformamide (100 ml) was added cesium carbonate (24.13 g, 74 mmol) lot wise under stirring at room temperature. After 16 hours, the reaction mixture was filtered through filter paper and filtrate was slowly poured into chilled water (700 ml) under stirring. After 30 minutes of stirring, the product was extracted with ethyl acetate (200 ml and 100 ml). The combined organic layers were washed with brine (100 ml). The organic extract was dried over anhydrous sodium sulfate and concentrated in vacuo to yield 12.5 g of the titled compound. The compound was purified by column chromatography using mixture of acetone and hexane as an eluent. The fractions containing the compound were collected and concentrated to provide 10 g of mixture of ethyl 1-{2-[(1,3-dioxo-1,3-dihydro-2H-isoindol-2-yl)oxy]ethyl}-1H-tetrazole-5-carboxylate and ethyl 2-{2-[(1,3-dioxo-1,3-dihydro-2H-isoindol-2-yl)oxy]ethyl}-2H-tetrazole-5-carboxylate in 53% yield.

Analysis:

Mass: 332(M+1); for Molecular weight: 331 and for Molecular formula: C₁₄H₁₃N₅O₅.

Step 2: Synthesis of mixture of ethyl 1-[2-(aminooxy)ethyl]-1H-tetrazole-5-carboxylate and ethyl 2-[2-(aminooxy)ethyl]-2H-tetrazole-5-carboxylate: To a solution of ethyl 1-{2-[(1,3-dioxo-1,3-dihydro-2H-isoindol-2-yl)oxy]ethyl}-1H-tetrazole-5-carboxylate and ethyl 2-{2-1(1,3-dioxo-1,3-dihydro-2H-isoindol-2-yl)oxylethyl}-2H-tetrazole-5-carboxylate (10 g, 3.02 mmol, product from step 1) in dichloromethane (100 ml) was added hydrazine hydrate (2.26 ml, 4.53 mmol) at 25-30° C. under stirring. The stirring was continued for 2 hours and the progress of reaction was monitored by thin layer chromatography using mixture of chloroform and methanol (9:1) as solvent. After complete conversion of starting material, the unwanted solid was filtered out and washed with dichloromethane (20 ml). The filtrate was concentrated at 40° C. under high vacuum to obtain 6.0 g of mixture of ethyl 1-[2-(aminooxy)ethyl]-1H-tetrazole-5-carboxylate and ethyl 2-[2-(aminooxy)ethyl]-2H-tetrazole-5-carboxylate (about 100% yield), the resulted compound was used in the next step without further purification.

### Preparation 41: Synthesis of 1-[2-(aminooxy)ethyl]-1H-tetrazole and 2-[2-(aminooxy)ethyl]-2H-tetrazole

Step 1: Synthesis mixture of 2-[2-(1H-tetrazol-1-yl)ethoxy]-1H-isoindole-1,3(2H)-dione and 2-[2-(2H-tetrazol-2-yl)ethoxy]-1H-isoindole-1,3(2H)-dione: To a solution of 2-(2-bromoethoxy)isoindoline-1,3-dione (5 g, 1.85 mmol, product from step 1 of preparation 10) and 1H-tetrazole (1.3 g, 1.85 mmol) in dimethylformamide (25 ml) was added cesium carbonate (6.0 g, 1.85 mmol) in a lot wise under stirring at room temperature. The progress of reaction was monitored by performing thin layer chromatography using mixture of acetone and hexane (3.5:6.5) as solvent. After 16 hours the completion of the reaction was confirmed by thin layer chromatography. The reaction mixture was slowly poured into chilled water (200 ml) under stirring. After stirring for 30 minutes, the precipitates formed were filtered and washed with water (50 ml). The solid compound was dried at 40° C. for 2 hours under high vacuum to provide 3.2 g of a mixture of 2-{2-(1H-tetrazol-1-yl)ethoxyl-1H-isoindole-1,3(2H)-dione and 2-{2-(2H-tetrazol-2-yl)ethoxy]-1H-isoindole-1,3(2H)-dione in 66% yield.

Analysis:

Mass: 260 (M+1) for Molecular weight: 259 and for Molecular formula: C₁₁H₉N₅O₃.

Step 2: Synthesis of 1-[2-(aminooxy)ethyl]-1H-tetrazole and 2-[2-(aminooxy)ethyl]-2H-tetrazole: To a flask containing mixture of 2-{2-(1H-tetrazol-1-yl)ethoxyl-1H-isoindole-1,3(2H)-dione and 2-{2-(2H-tetrazol-2-yl)ethoxyl-1H-isoindole-1,3(2H)-dione (3.0 g, 1.15 mmol, product from step 1) in dichloromethane (30 ml) was added hydrazine hydrate (0.870 ml, 1.73 mmol) at 25-30° C. under stirring and continued stirring for 2 hours. The progress of reaction was monitored by thin layer chromatography. After complete conversion, the unwanted solid was filtered out and washed with dichloromethane (12 ml). The filtrate was concentrated at 40° C. under high vacuum to obtain 1.5 g of crude mixture of 1-[2-(aminooxy)ethyl]-1H-tetrazole and 2-[2-(aminooxy)ethyl]-2H-tetrazole (about 100% yield) and used without further purification in the next step.

### Preparation 42: Synthesis of 2-[2-(aminooxy)ethyl]-5-methyl-2H-tetrazole

Step 1: Synthesis of mixture of 2-[2-(5-methyl-1H-tetrazol-1-yl)ethoxy]-1H-isoindole-1,3(2H)-dione and 2-[2-(5-methyl-2H-tetrazol-2-yl)ethoxy]-1H-isoindole-1,3(2H)-dione: To a solution of 2-(2-bromoethoxy)isoindoline-1,3-dione (20 g, 7.4 mmol prepared according to the procedure described in step 1 of preparation 10) and 5-methyl-1H-tetrazole (6.22 g, 7.4 mmol) in dimethylformamide (100 ml) was added cesium carbonate (24.13 g, 7.4 mmol) in a lot wise under stirring at 25-30° C. After 16 hours, completion of the reaction was confirmed by performing the thin layer chromatography using mixture of chloroform and methanol as solvent. The resulted reaction mixture was filtered and the filtrate was slowly poured into chilled water (700 ml) under stirring. After 30 minutes of stirring, the reaction mixture was extracted with ethyl acetate (200 ml and 100 ml). The combined organic layer was washed with brine (100 ml). The organic layer was dried on sodium sulfate and concentrated on rota vapour to provide 18 g of the crude product. The two regio isomers were separated by using column chromatography using mixture of acetone and hexane as an eluent. The upper spot isolated at 15-25% concentration of acetone in hexane yielded 7.0 g of non-polar isomer 2-[2-(5-methyl-2H-tetrazol-2-yl)ethoxy]-1H-isoindole-1,3(2H)-dione. The lower spot isolated at 25-40% concentration of acetone in hexane yielded 3.8 g of polar isomer 2-[2-(5-methyl-1H-tetrazol-1-yl)ethoxy]-1H-isoindole-1,3 (2H)-dione. (10.8 g, 53% yield).

Analysis:

Mass: 274.2 (M+1); for Molecular weight: 273 and Molecular formula: C₁₂H₁₁N₅O₃.

Step 2: Synthesis of 2-[2-(aminooxy)ethyl]-5-methyl-2H-tetrazole: To a solution of 2-[2-(5-methyl-2H-tetrazol-2-yl)ethoxyl-1H-isoindole-1,3(2H)-dione (3.8 g, 1.39 mmol) in dichloromethane (38 ml) was added hydrazine hydrate (1.05 ml, 2.08 mmol) at 25-30° C. under stirring. After 2 hours, the progress of reaction was monitored by thin layer chromatography using mixture of chloroform and methanol (9:1). After complete conversion of starting material, unwanted solid was filtered out and washed with dichloromethane (7.6 ml). The filtrate was concentrated at 40° C. under high vacuum to obtain 1.99 g of 2-[2-aminooxy)ethyl]-5-methyl-2H-tetrazole, which was used without further purification in the next step.

### Preparation 43: Synthesis of 2-[2-aminooxy)ethyl]-5-methyl-1H-tetrazole

To a solution of 2-[2-(5-methyl-1H-tetrazol-1-yl)ethoxy]-1H-isoindole-1,3 (2H)-dione (1.8 g, 0.659 mmol, product from step 1 of Preparation 13) in dichloromethane (18 ml) was added hydrazine hydrate (0.494 ml, 0.989 mmol) at 25-30° C. under stirring. After 2 hours, the progress of reaction was monitored by thin layer chromatography using mixture of chloroform and methanol (9:1). After complete conversion of starting material, unwanted solid was filtered out and washed with dichloromethane (7.6 ml). The filtrate was concentrated at 40° C. under high vacuum to obtain 942 mg of 2-[2-aminooxy)ethyl]-5-methyl-1H-tetrazole, which was used in the next step without further purification.

### Preparation 44: Synthesis of ethyl {2-[2-(aminooxy)ethyl]-2H-tetrazol-5-yl}acetate

Step-1: Synthesis mixture of ethyl (2-{2-[(1,3-dioxo-1,3-dihydro-2H-isoindol-2-yl)oxy]ethyl}-2H-tetrazol-5-yl)acetate and ethyl (1-{2-[(1,3-dioxo-1,3-dihydro-2H-isoindol-2-yl)oxy]ethyl}-1H-tetrazol-5-yl)acetate: To a mixture of 2-(2-bromoethoxy)isoindoline-1,3-dione (20 g, 74 mmol) and ethyl 1H-tetrazol-5-ylacetate (11.55 g, 74 mmol) in DMF (100 ml) was added cesium carbonate (29 gm, 0.088 mol) in portion wise at 25-30° C. under stirring. The stirring was continued for 16 hrs. The reaction mixture was filtered and the collected filtrate was slowly poured onto chilled water (700 ml) under stirring. The formed precipitates of compound were filtered on Buchner funnel and washed with water (70 ml). The solid compound was dried at 40° C. for 2 hour under reduced pressure to yield crude compound 18.0 g. The crude compound was column purified using hexane and acetone. The non-polar spot isolated at 10-15% concentration of acetone in hexane and yielded 8.0 g of ethyl (2-{2-[(1,3-dioxo-1,3-dihydro-2H-isoindol-2-yl)oxy]ethyl}-2H-tetrazol-5-yl)acetate. The polar spot isolated at 20-25% concentration of acetone in hexane, and the pure fractions were collected and concentrated on rota evaporator which yielded 5.0 g of ethyl (1-{2-[(1,3-dioxo-1,3-dihydro-2H-isoindol-2-yl)oxy]ethyl}-1H-tetrazol-5-yl)acetate as white solid in 53% yield.

Analysis:

Mass: 346.3 (M+H); for Molecular weight: 345 and Molecular formula: C₁₅H₁₅N₅O₅.

¹HNMR (CDCl₃): δ 7.84-7.26 (m, 4H), 5.05 (t, 2H, J=5.2 Hz), 4.77 (t, 2H, J=5.6 Hz), 4.23-4.18 (q, 2H), 3.97 (s, 2H), 1.27 (t, 3H, J=7.2 Hz).

Step-2: Hydrazine hydrate monohydrate (0.870 ml, 17.3 mmol) was added to ethyl (2-{2-[(1,3-dioxo-1,3-dihydro-2H-isoindol-2-yl)oxy]ethyl}-2H-tetrazol-5-yl) acetate (4.0 g, 11.5 mmol, non-polar product from step 1) in DCM (40 ml) at room temperature under stirring and continued stirring for 2 hr. Progress of reaction was monitored by TLC. The Unwanted solid was filtered out and washed with DCM (25 ml) and filtrate was concentrated at 40° C. under reduced pressure, yielded 2.49 g crude compound and used without further purification.

Analysis: Mass: 216.3 (M+1); for Molecular weight: 215 and Molecular formula: C₇H₁₃N₅O₃.

### Preparation 45: Synthesis of 2-[2-(aminooxy)ethyl]-5-(2-tertiary-butyl-dimethyl-silanyloxy ethyl)-2H-tetrazole

Step-1: Synthesis mixture of 2-{2-[5-(2-tertiary-butyl-dimethyl-silanyloxy ethyl)-1H-tetrazol-1-yl]ethoxyl-1H-isoindole-1,3(2H)-dione and 2-{2-[5-(2-tertiary-butyl-dimethyl-silanyloxy ethyl)-2H-tetrazol-2-yl]ethoxy}-1H-isoindole-1,3(2H)-dione: To a solution of 2-(2-bromoethoxy)isoindoline-1,3-dione (20 g, 74 mmol) in dimethylformamide (100 ml) added 5-(2-tertiary-butyl-dimethyl-silanyloxy ethyl)-1H-tetrazole (14.81 g, 74 mmol) at 25-30° C. under stirring followed by cesium carbonate ((29 g, 88 mmol) in portion wise under stirring. The reaction was continued further at same temperature for 16 hrs and filtered. The filtrate was slowly poured onto chilled water (700 ml) under stirring and continued stirring for 30 minutes. The formed precipitates were filtered and washed with water (70 ml). The solid compound was dried at 40° C. for 2 hour under high vacuum to yield crude compound 17.2 g. The crude compound was column purified (silica gel 60-120 mesh size) using hexane and acetone as an eluent. The non-polar spot isolated at 8-12% concentration of acetone in hexane, yielded 8 g of 2-{2-[5-(2-tertiary-butyl-dimethyl-silanyloxy ethyl)-2H-tetrazol-2-yl]ethoxy}-1H-isoindole-1,3 (2H)-dione. The polar spot was isolated at 16-20% concentration of acetone in hexane and yielded 4.2 g of 2-{2-[5-(2-tertiary-butyl-dimethyl-silanyloxy ethyl)-1H-tetrazol-1-yl]ethoxy}-1H-isoindole-1,3 (2H)-dione as white solid.

Analysis:

Mass: 418.3 (M+H); for Molecular weight: 417 and Molecular formula: C₁₉H₂₇N₅O₄Si.

¹HNMR (CDCl₃): δ 7.83-7.74 (m, 4H), 5.00 (t, 2H, J=6 Hz), 4.73 (t, 2H, J=6 Hz), 3.98 (t, 2H, J=6.8 Hz), 3.08 (t, 2H, J=7.2 Hz), 0.837 (s, 9H), 0.002 (s, 6H).

Step-2: Synthesis of 2-[2-(aminooxy)ethyl]-5-(2-tertiary-butyl-dimethyl-silanyloxy ethyl)-2H-tetrazole: Hydrazine hydrate monohydrate (0.719 ml, 14.3 mmol) was added to solution of 2-{2-[5-(2-tertiary-butyl-dimethyl-silanyloxy ethyl)-2H-tetrazol-2-yl]ethoxy}-1H-isoindole-1,3 (2H)-dione (4.0 g, 9.5 mmol) in dichloromethane (40 ml) at ambient temperature under stirring and continued stirring for 2 hour. Progress of reaction was monitored by TLC (ethyl acetate hexane, 1:1). After complete consumption of starting material the unwanted solid was filtered out and washed with dichloromethane (25 ml). The collected filtrate was concentrated at 40° C. under high vacuum and yielded 2.75 g of 2-[2-(aminooxy)ethyl]-5-(2-tertiary-butyl-dimethyl-silanyloxy ethyl)-2H-tetrazole, which was used for next step without purification.

Analysis:

Mass: 288.3 (M+1); for Molecular weight: 287 and Molecular formula: C₁₁H₂₅N₅O₂Si.

### Preparation 46: Synthesis of 1-[2-(aminooxy)ethyl]-5-(2-tertiary-butyl-dimethyl-silanyloxy ethyl)-1H-tetrazole

Hydrazine hydrate monohydrate (0.719 ml, 14.3 mmol) was added to 2-{2-[5-(2-tertiary-butyl-dimethyl-silanyloxy ethyl)-1H-tetrazol-1-yl]ethoxy}-1H-isoindole-1,3 (2H)-dione (4.0 g, 9.5 mmol, polar compound prepared as per step 1 of preparation 45) in dichloromethane (40 ml) at 25-30° C. under stirring. After completion of 2 hours of stirring the completion of reaction was confirmed by performing the TLC and the unwanted solid was filtered, washed with dichloromethane (25 ml). The collected filtrate was concentrated at 40° C. under high reduced pressure and yielded 2.70 g of the product, which was used in the next step without further purification.

Analysis:

Mass: 288.3 (M+1); for Molecular weight: 287 and Molecular formula: C₁₁H₂₅N₅O₂Si.

### Preparation 47: Synthesis of 2-[2-(aminooxy)ethyl]-4-(tertiary-butyl-dimethyl-silanyloxymethyl)-2H-1,2,3-triazole

Step 1: Synthesis and separation of a mixture of 2-{2-[4-(tertiary-butyl-dimethyl-silanyloxy methyl)-2H-1,2,3-triazol-2-yl]ethoxyl-1H-isoindole-1,3(2H)-dione, 2-{2-[4-(tertiary-butyl-dimethyl-silanyloxymethyl)-1H-1,2,3-triazol-1-yl]ethoxyl-1H-isoindole-1,3(2H)-dione and 2-{2-[5-(tertiary-butyl-dimethyl-silanyloxymethyl)-1H-1,2,3-triazol-1-yl]ethoxyl-1H-isoindole-1,3(2H)-dione: To a solution of 4-tertiary-butyl-dimethyl-silanyloxy methyl-2H-1,2,3-triazol (40 g, 187 mmol, prepared according to the procedure described in US20150266867) in dimethylformamide (200 ml) was added potassium carbonate (28.4 g, 205 mmol) under stirring. After completion of 15 minutes of stirring a solution of 2-(2-bromoethoxy)-1H-isoindole-1,3(2H)-dione (50.5 g, 187 mmol, prepared according to the procedure described in PCT Int. Appl. 2000018790) was added. Continued stirring with continuous monitoring of the reaction. The progress of reaction was monitored by TLC (hexane: ethyl acetate, 5:5). After complete consumption of starting material the reaction mixture was filtered through celite bed to remove insolubles and filtrate was concentrated. The concentrated mass was added to water (400 ml) and extracted with ethyl acetate (2×400 ml). The organic extracts were combined and washed with brine (200 ml), dried over anhydrous sodium sulphate. The volatiles were removed under reduced pressure yielded 67 g crude compound. The isomers were separated by column chromatography (100-200 mesh size silica gel) using hexane: ethyl acetate, 8:2 as an eluent. The non-polar isomer 2-{2-[4-(tertiary-butyl-dimethyl-silanyloxy methyl)-2H-1,2,3-triazol-2-yl]ethoxy}-1H-isoindole-1,3(2H)-dione was eluted at 12% ethyl acetate in hexane and yielded 24 g of the product. The mixture of polar isomers 2-{2-[4-(tertiary-butyl-dimethyl-silanyloxymethyl)-1H-1,2,3-triazol-1-yl]ethoxy}-1H-isoindole-1,3 (2H)-dione and 2-{2-[5-(tertiary-butyl-dimethyl-silanyloxymethyl)-1H-1,2,3-triazol-1-yl]ethoxy}-1H-isoindole-1,3(2H)-dione was eluted at 45% ethyl acetate in hexane to obtain 12 g of the product.

Step 2: Synthesis of 2-[2-(aminooxy)ethyl]-4-(tertiary-butyl-dimethyl-silanyloxymethyl)-2H-1,2,3-triazole: To a solution 2-{2-[4-(tertiary-butyl-dimethyl-silanyloxy methyl)-2H-1,2,3-triazol-2-yl]ethoxy}-1H-isoindole-1,3(2H)-dione (8.7 g, 0.0216 moles, obtained from step 1) in dichloromehane (43.5 ml) was added hydrazine hydrate monohydrate (1.6 ml, 0.032 moles) at 25-30° C. under stirring. The progress of reaction was monitored by TLC (chloroform: methanol, 9:1). After complete consumption of starting material the reaction mixture was filtered on Buchner funnel and unwanted solid was washed with dichloromethane (10 ml) and filtrate was concentrated under reduced pressure to yield 6 g of 2-[2-(aminooxy)ethyl]-4-(tertiary-butyl-dimethyl-silanyloxymethyl)-2H-1,2,3-triazole as oily mass, which was used in next reaction without purification.

### Example 1

### Synthesis of Di sodium (2S,5R)-7-oxo-6-(sulfooxy)-N′-(H-tetrazol-5-ylcarbonyl)-1,6-diazabicyclo[3.2.1]octane-2-carbohydrazide

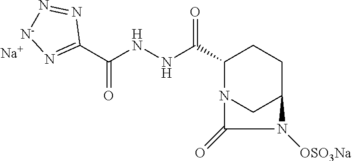

Step 1: Synthesis of ethyl 1-(4-nitrobenzyl)-1H-tetrazole-5-carboxylate and ethyl 2-(4-nitrobenzyl)-2H-tetrazole-5-carboxylate: To a clean dry flask was charged ethyl-1H-tetrazole-5-carboxylate (Synthesis, 45(8), 1051-1059; 2013) (5 g, 35.2 mmol) in dimethylformamide (50 ml) to get a clear solution at 25-30° C. To this was charged potassium carbonate (5.34 g, 38.7 mmol) under stirring; effervescence was observed. After 10 minutes of stirring, 4-nitro benzyl bromide (7.22 g, 33.40 mmol) was added. The completion of reaction was monitored by performing thin layer chromatography using mixture of chloroform and methanol (9:1) as solvent. After completion of the reaction, to the reaction mixture was added to water (500 ml) and extracted with ethyl acetate (2×250 ml). The combined organic extract was washed with brine (1×100 ml), dried over anhydrous sodium sulfate and concentrated under reduced pressure to get 5.8 g of required product as a mixture of ethyl 1-(4-nitrobenzyl)-1H-tetrazole-5-carboxylate and ethyl 2-(4-nitrobenzyl)-2H-tetrazole-5-carboxylate as reddish thick oil in 59.48% yield.

Step 2: Synthesis of 1-(4-nitrobenzyl)-1H-tetrazole-5-carbohydrazide and 2-(4-nitrobenzyl)-2H-tetrazole-5-carbohydrazide: To a solution of mixture of ethyl 1-(4-nitrobenzyl)-1H-tetrazole-5-carboxylate and ethyl 2-(4-nitrobenzyl)-2H-tetrazole-5-carboxylate (5.8 g, 22.0 mmol, product obtained in step 1) in ethanol (60 ml) was added hydrazine hydrate (1.10 g, 22.0 mmol) under stirring at 25-30° C. The progress of reaction was monitored by thin layer chromatography using mixture of chloroform and methanol (9:1). After complete consumption of starting material, the volatiles were removed under reduced pressure to get yellowish residue. To this was added diethyl ether (100 ml) and stirred until precipitates were obtained. The obtained precipitates were filtered and washed with ether (2×25 ml) to get 2.8 g of the required product as mixture of 1-(4-nitrobenzyl)-1H-tetrazole-5-carbohydrazide and 2-(4-nitrobenzyl)-2H-tetrazole-5-carbohydrazide in 50% yield.

Step 3: Synthesis of (2S,5R)-6-(benzyloxy)-7-oxo-N′-(1-(4-nitrobenzyl)-1H-tetrazol-5-ylcarbonyl)-1,6-diazabicyclo[3.2.1]octane-2-carbohydrazide and (2S,5R)-6-(benzyloxy)-7-oxo-N′-(2-(4-nitrobenzyl)-2H-tetrazol-5-ylcarbonyl)-1,6-diazabicyclo[3.2.1]octane-2-carbohydrazide: To a suspension of 1-(4-nitrobenzyl)-1H-tetrazole-5-carbohydrazide and 2-(4-nitrobenzyl)-2H-tetrazole-5-carbohydrazide (2.8 g, 10.5 mmol, the product obtained in Step 2) in water (100 ml) was added sodium (2S,5R)-6-(benzyloxy)-7-oxo-1,6-diazabicyclo[3.2.1[octane-2-carboxylate (3.13 g, 10.5 mmol, prepared as per the procedure disclosed in International Patent Application No. PCT/IB2013/059264) under stirring. To this was added EDC.HCl (4.02 g, 21 mmol) followed by hydroxy benzotriazole (1.42 g, 10.5 mmol) under stirring. The progress of the reaction was monitored by thin layer chromatography using mixture of chloroform and methanol (9:1). After complete consumption of starting material, dichloromethane (100 ml) was added under stirring. The organic extract was separated and aqueous layer was re-extracted with dichloromethane (1×50 ml). The organic extracts were combined and washed with 5% potassium hydrogen sulfate solution (1×50 ml), saturated sodium hydrogen carbonate solution (1×50 ml) and finally with brine (1×50 ml). The volatiles were removed under reduced pressure to get 3.8 g of the crude product. This was purified by column chromatography using 100-200 mesh silica gel and mixture of chloroform and methanol (9:1) as solvent. The fractions containing products were collected and concentrated under reduced pressure to provide 2.3 g of mixture of (2S,5R)-6-(benzyloxy)-7-oxo-N′-(1-(4-nitrobenzyl)-1H-tetrazol-5-ylcarbonyl)-1,6-diazabicyclo[3.2.1]octane-2-carbohydrazide and (2S,5R)-6-(benzyloxy)-7-oxo-N-(2-(4-nitrobenzyl)-2H-tetrazol-5-ylcarbonyl)-1,6-diazabicyclo[3.2.1]octane-2-carbohydrazide in 42% yield.

Step 4: (2S,5R)-6-Hydroxy-7-oxo-N′-(1H-tetrazol-5-ylcarbonyl)-1,6-diazabicyclo[3.2.1]octane-2-carbohydrazide/2S,5R)-6-Hydroxy-7-oxo-N′-(2H-tetrazol-5-ylcarbonyl)-1,6-diazabicyclo[3.2.1]octane-2-carbohydrazide: To a stirred solution of (2S,5R)-6-(benzyloxy)-7-oxo-N-(1-(4-nitrobenzyl)-1H-tetrazol-5-ylcarbonyl)-1,6-diazabicyclo[3.2.1]octane-2-carbohydrazide and (2S,5R)-6-(benzyloxy)-7-oxo-N-(2-(4-nitrobenzyl)-2H-tetrazol-5-ylcarbonyl)-1,6-diazabicyclo[3.2.1]octane-2-carbohydrazide (2.3 g, 4.4 moles, product obtained in step 3) in methanol (25 ml) and water (25 ml) was added ammonium formate (1.4 g, 22 mmol) and 10% palladium over carbon (230 mg). The reaction mixture was heated at 50-55° C. The progress of reaction was monitored by thin layer chromatography using mixture of chloroform and methanol (9:1) as solvent. The catalyst was removed by filtration on celite bed, washed with methanol (2×10 ml). The filtrate was concentrated under reduced pressure to obtain 900 mg of (2S,5R)-6-hydroxy-7-oxo-N-(1H-tetrazol-5-ylcarbonyl)-1,6-diazabicyclo[3.2.1]octane-2-carbohydrazide and (2S,5R)-6-hydroxy-7-oxo-N′-(2H-tetrazol-5-ylcarbonyl)-1,6-diazabicyclo[3.2.1]octane-2-carbohydrazide as yellowish solid in 69% yield.

Step 5: Di tetrabutyl ammonium salt of (2S,5R)-7-oxo-6-sulfooxy-N′-(H-tetrazol-5-ylcarbonyl)-1,6-diazabicyclo[3.2.1]octane-2-carbohydrazide: To a stirred solution of (2S,5R)-6-hydroxy-7-oxo-N′-(1H-tetrazol-5-ylcarbonyl)-1,6-diazabicyclo[3.2.1]octane-2-carbohydrazide and (2S,5R)-6-hydroxy-7-oxo-N′-(2H-tetrazol-5-ylcarbonyl)-1,6-diazabicyclo[3.2.1]octane-2-carbohydrazide (900 mg, 3.0 mmol, product obtained in step 4) in pyridine (25 ml) was added pyridine sulfur trioxide complex (1.45 g, 9.1 mmol). The reaction mixture was stirred at 30-35° C. The progress of reaction was monitored by mass spectroscopy. The insoluble reagent was removed by filtration on celite bed, washed with pyridine (2×10 ml). Filtrate was concentrated under reduced pressure the residue obtained was taken in 5% potassium dihydrogen phosphate solution (100 ml) and stirred for 1 hour. The solution was washed with ethyl acetate (2×100 ml). The aqueous reaction mixture was taken in flask and tetra butyl ammonium sulfate (1.032 g, 3.0 mmol) was added to it under stirring. The reaction mixture was stirred for 3 hours and extracted with dichloromethane (2×50 ml). The dichloromethane extract was dried over anhydrous sodium sulfate and volatiles were removed under reduced pressure to get 950 mg di tetrabutyl ammonium salt of (2S,5R)-7-oxo-6-sulfooxy-N-(H-tetrazol-5-ylcarbonyl)-1,6-diazabicyclo[3.2.1]octane-2-carbohydrazide in 31% yield.

Step 6: Di sodium salt of (2S,5R)-7-oxo-6-sulfooxy-N′-(H-tetrazol-5-ylcarbonyl)-1,6-diazabicyclo[3.2.1]octane-2-carbohydrazide: To appropriate column was charged 80 g Sodium Resin (INDION 225 Na⁺ resin) and eluted with HPLC grade water (500 ml) followed by 10% tetrahydrofuran in water (500 ml). Di tetrabutyl ammonium salt of (2S,5R)-7-oxo-6-sulfooxy -N′-(1H-tetrazol-5-ylcarbonyl)-1,6-diazabicyclo[3.2.1]octane-2-carbohydrazide/Di tetrabutyl ammonium salt of (2S,5R)-7-oxo-6-sulfooxy-N-(2H-tetrazol-5-ylcarbonyl)-1,6-diazabicyclo[3.2.1]octane-2-carbohydrazide (800 mg) dissolved in minimum tetrahydrofuran (4 ml) was charged on column. The column was eluted with 10% tetrahydrofuran in water and fractions of each 50 ml were collected. The fractions containing Di sodium salt of (2S,5R)-7-oxo-6-sulfooxy-N′-(1H-tetrazol-5-ylcarbonyl)-1,6-diazabicyclo[3.2.1]octane-2-carbohydrazide or Di sodium salt of (2S,5R)-7-oxo-6-sulfooxy-N-(2H-tetrazol-5-ylcarbonyl)-1,6-diazabicyclo[3.2.1]octane-2-carbohydrazides were collected and concentrated under reduced pressure to remove tetrahydrofuran. The aqueous eluent was washed with dichloromethane (2×100 ml) and concentrated under reduced pressure to obtain 200 mg of Di sodium salt of (2S,5R)-7-oxo-6-sulfooxy-N-(1H-tetrazol-5-ylcarbonyl)-1,6-diazabicyclo[3.2.1]octane-2-carbohydrazide or (2S,5R)-7-oxo-6-sulfooxy-N-(2H-tetrazol-5-ylcarbonyl)-1,6-diazabicyclo[3.2.1]octane-2-carbohydrazide as white solid in 49% yield. It was further purified by using water and isopropanol mixture by adding isopropanol (700 μl) to a solution of Di sodium salt of (2S,5R)-7-oxo-6-sulfooxy-N-(2H-tetrazol-5-ylcarbonyl)-1,6-diazabicyclo[3.2.1]octane-2-carbohydrazide or (2S,5R)-7-oxo-6-sulfooxy-N-(1H-tetrazol-5-ylcarbonyl)-1,6-diazabicyclo[3.2.1]octane-2-carbohydrazide (100 mg, 0.23 mmol) in water (100 μl) under stirring. Stirring was continued until salts precipitated out. After three days the precipitated solids were filtered and washed with isopropanol (2×500 μl). The product obtained was dried under reduced pressure to get 48 mg of the desired product as off-white solid in 48% yield.

Analysis:

Melting Point: 166-168° C. (decomposes);

Mass: 375.2 (M−1), 377 (M+1) for free acid; for Molecular weight: 420 and Molecular formula: C₉H₁₀N₈O₇S.2Na;

¹HNMR (400 MHz, DMSO-d₆): δ 4.00(m, 1H), 3.83 (m, 1H), 3.33 (m, 1H), 3.03 (m, 1H), 2.05-2.02 (m, 1H), 1.85 (m, 1H), 1.72 (m, 2H);

Purity as determined by HPLC: 93.84%.

### Example 2

### Sodium salt of (2S,5R)-N′-(1H-imidazol-1-ylacetyl)-7-oxo-6-(sulfooxy)-1,6-diazabicyclo[3.2.1]octane-2-carbohydrazide

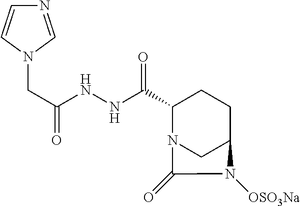

Step 1: Synthesis of ethyl 1H-imidazol-1-ylacetate: To a solution of imidazole (25 g, 367 mmol) in dimethylformamide (125 ml) was added potassium carbonate (60.8 g, 0.440 mol) under stirring at 25-30° C.; effervescence was observed. After stirring for 10 minutes, ethyl bromoacetate (64.3 g, 385 mmol) was added to the reaction mixture under continuous stirring. The completion of reaction was monitored by thin layer chromatography using mixture of chloroform and methanol (9:1) as solvent. After completion of the reaction, the reaction mixture was added to water (750 ml) and extracted with dichloromethane (4×250 ml). The combined organic extract was washed with brine (1×250 ml), dried over anhydrous sodium sulfate and concentrated under reduced pressure to provide 24 g of ethyl 1H-imidazol-1-ylacetate as thick reddish oil in 42.34% yield.

Step 2: Synthesis of 2-(1H-imidazol-1-yl)acetohydrazide: To a clean dry flask was charged ethyl 1H-imidazol-1-ylacetate (15 g, 97.2 mmol, product obtained in step 1) in ethanol (75 ml). The hydrazine hydrate (5.35 g, 106 mmol) was added to it at 25-30° C. under stirring and the progress of reaction was monitored by thin layer chromatography using mixture of chloroform and methanol (9:1) as solvent. After complete consumption of starting material the volatiles were removed under reduced pressure to provide yellowish residue. To this was added diethyl ether (100 ml) and stirred to get precipitates of required compound. The obtained product was filtered and washed with ether (2×50 ml) to get 9.0 g of 2-(1H-imidazol-1-yl)acetohydrazide in 66.03% yield. The obtained product was used without further purification.

Step 3: Synthesis of (2S,5R)-N′-(1H-imidazol-1-ylacetyl)-6-benzyloxy-7-oxo-1,6-diazabicyclo[3.2.1]octane-2-carbohydrazide: To a solution of sodium (2S,5R)-6-(benzyloxy)-7-oxo-1,6-diazabicyclo[3.2.1]octane-2-carboxylate (17 g, 57.1 mmol, prepared as per the procedure disclosed in International Patent Application No. PCT/IB2013/059264) was added EDC.HCl (24 g, 125 mmol) after 15 minutes stirring solid 2-(1H-imidazol-1-yl)acetohydrazide (8 g, 57.1 mmol) was added followed by charging of 1-hydroxy benzotriazole (8.73 g, 57.1 mmol) under stirring. The progress of reaction was monitored by thin layer chromatography using mixture of chloroform and methanol (9:1) as solvent. After complete consumption of starting material, the precipitates obtained were filtered and washed with water (2×100 ml) to yield 15 g of (2S,5R)-N′-(1H-imidazol-1-ylacetyl)-6-bezyloxy-7-oxo-1,6-diazabicyclo[3.2.1]octane-2-carbohydrazide as white solid in 65.9% yield.

Step 4: Synthesis of (2S,5R)-6-hydroxy-N′-(1H-imidazol-1-ylacetyl)-7-oxo-1,6-diazabicyclo[3.2.1]octane-2-carbohydrazide: To a stirred solution of (2S,5R)-N′-(1H-imidazol-1-ylacetyl)-6-bezyloxy-7-oxo-1,6-diazabicyclo[3.2.1]octane-2-carbohydrazide (14 g, 35.1 mmol, product from step 3) in methanol (140 ml) was added 10% palladium over carbon (3.5 g). To this suspension was purged hydrogen gas continuously. The progress of the reaction was monitored by thin layer chromatography using mixture of chloroform and methanol (9:1) as solvent. After completion of the reaction the catalyst was removed by filtration on celite bed, washed with methanol (2×28 ml). The filtrate was concentrated under reduced pressure to provide 10.3 g of (2S,5R)-6-hydroxy-N-(1H-imidazol-1-ylacetyl)-7-oxo-1,6-diazabicyclo[3.2.1]octane-2-carbohydrazide as white solid in 95.4% yield.

Step 5: Synthesis of tetrabutyl ammonium salt of (2S,5R)-N′-(1H-imidazol-1-ylacetyl)-7-oxo-6-(sulfooxy)-1,6-diazabicyclo[3.2.1]octane-2-carbohydrazide: To a stirred solution of (2S,5R)-6-hydroxy-N′-(1H-imidazol-1-ylacetyl)-7-oxo-1,6-diazabicyclo[3.2.1]octane-2-carbohydrazide (10.3 g, 33.4 mmol, product obtained in step 4) in dimethylformamide (70 ml) was added dimethylformamide sulfur trioxide complex (6.45 g, 42.1 mmol). The reaction mixture was stirred at 10-15° C. The progress of reaction was monitored by thin layer chromatography using mixture of chloroform and methanol (8:2) as solvent. After completion of the reaction, solution of tetrabutyl ammonium acetate (12.69 g, 42 mmol) in water (43 ml) was added to the reaction mixture under stirring. After complete conversion to tetrabutylammoinum salt, the volatiles were removed under vacuum at 40-45° C. The obtained residue was partitioned in mixture of dichloromethane and water (140 ml+140 ml). The organic layer was dried over anhydrous sodium sulfate and distilled to get 23 g of crude product. The crude product was purified by column chromatography using 100-200 mesh silica gel using mixture of chloroform and methanol as an eluent. The pure fractions were collected and were concentrated to obtain 12 g of tetrabutyl ammonium salt of (2S,5R)-N′-(1H-imidazol-1-ylacetyl)-7-oxo-6-(sulfooxy)-1,6-diazabicyclo[3.2.1]octane-2-carbohydrazide in 54.15% yield.

Step 6: Sodium salt of (2S,5R)-N′-(1H-imidazol-1-ylacetyl)-7-oxo-6-(sulfooxy)-1,6-diazabicyclo[3.2.1]octane-2-carbohydrazide: To appropriate column was charged 660 g Sodium Resin (INDION 225 Na⁺ resin) and eluted with HPLC grade water (2000 ml) followed by 10% tetrahydrofuran in water. Tetrabutyl ammonium salt of (2S,5R)-N′-(1H-imidazol-1-ylacetyl)-7-oxo-6-(sulfooxy)-1,6-diazabicyclo[3.2.1]octane-2-carbohydrazide (11 g, product from step 5) dissolved in minimum tetrahydrofuran (11 ml) was charged on to the column. The column was eluted with 10% tetrahydrofuran in water and fractions of 50 ml were collected. The fractions containing product were collected and concentrated under reduced pressure to remove tetrahydrofuran. The aqueous eluent was washed with dichloromethane (2×100 ml) and concentrated under reduced pressure to yield 5.4 g of sodium salt of (2S,5R)-N′-(1H-imidazol-1-ylacetyl)-7-oxo-6-(sulfooxy)-1,6-diazabicyclo[3.2.1]octane-2-carbohydrazide in 75.52% yield.

Analysis:

Mass: 387.3 (M−H) 389.3(M+H) for free acid, for the Molecular weight: 410.33 and for Molecular formula: C₁₂H₁₅N₆O₇S.Na;

¹H NMR (DMSO-d₆): δ 10.1 (2H, br s), 7.59 (1H, s), 7.10 (1H, s), 6.87(1H, s), 4.72 (2H, s), 3.99 (1H, s), 3.83 (1H, d, J=8 Hz), 3.18 (1H, d, J=12Hz), 2.98 (1H, d, J=12 Hz), 2.03-1.99 (1H, m), 1.77-1.59 (3H, m).

Purity as determined by HPLC: 94.03%.

### Example 3

### Synthesis of sodium salt of (2S,5R)-6-(sulfooxy)-7-oxo-N′-(1H-tetrazol-1-ylacetyl)-1,6-diazabicycle[3.2.1]octane-2-carbohydrazide

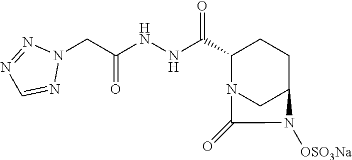

Step 1: Synthesis of benzyl 1H-tetrazol-1-ylacetate and benzyl 2H-tetrazol-2-ylacetate: To a three neck flask equipped with overhead stirrer was added a solution of 1-H-tetrazole (25 g, 357 mmol) in dimethylformamide (250 ml) at 25-30° C. To this was added potassium carbonate (49 g, 357 mmol) under stirring; effervescence was observed. Benzyl bromoacetate (81.39 g, 357 mmol) was added to reaction mixture drop-wise using addition funnel at 25-30° C. The progress of reaction was monitored by thin layer chromatography using a mixture of chloroform and methanol (9:1). After complete consumption of starting material the reaction mixture was filtered through celite bed to remove insoluble inorganics and filtrate was added to water (2500 ml). The aqueous reaction mixture was extracted with ethyl acetate (3×500 ml). The organic extracts were combined and washed with brine (1×500 ml) and then dried over anhydrous sodium sulfate. The volatiles were removed under reduced pressure to provide 70 g of mixture of benzyl 1H-tetrazol-1-ylacetate and benzyl 2H-tetrazol-2-ylacetate in 90% yield.

Step 2: Synthesis of 1H-tetrazol-1-ylacetic acid and 2H-tetrazol-2-ylacetic acid: To the solution of benzyl 1H-tetrazol-1-ylacetate and benzyl 2H-tetrazol-2-ylacetate (70 g, 321 mmol, the product from Step 1) in tetrahydrofuran (700 ml) was added 10% palladium over carbon (7 g) at 50 psi hydrogen gas. The progress of reaction was monitored by thin layer chromatography using mixture of chloroform and methanol (9:1). After complete consumption of starting material the reaction mixture was filtered on celite bed and washed with tetrahydrofuran (2×140 ml). The filtrate was concentrated under reduced pressure to provide 25 g of mixture of 1H-tetrazol-1-ylacetic acid and 2H-tetrazol-2-ylacetic acid in 60% yield.

Step 3: Synthesis of benzyl 2-(1H-tetrazol-1-ylacetyl)hydrazinecarboxylate and benzyl 2-(2H-tetrazol-2-ylacetyl)hydrazinecarboxylate: To a clean flask containing a solution of 1H-tetrazol-1-ylacetic acid and 2H-tetrazol-2-ylacetic acid (25 g, 195 mmol, product from Step 2) in dimethylformamide (250 ml) was added benzyl hydrazinecarboxylate (32.4 g, 195 mmol) at 25-30° C. To this solution was added EDC.HCl (56 g, 292 mmol) and hydroxy benzotriazole (26.36 g, 195 mmol) under stirring. Finally N-methyl morpholine (59 g, 585 mmol) was added to it. The progress of reaction was monitored by thin layer chromatography using mixture of chloroform and methanol (9:1). After complete consumption of starting material the reaction was quenched by adding water (2.5 l) and then extracted with ethyl acetate (3×500 ml). The organic extracts were combined and washed with potassium hydrogen sulfate solution (1×250 ml), saturated sodium hydrogen carbonate solution (1×250 ml), brine (1×250 ml). The volatiles were removed under reduced pressure to get 40 g of mixture of benzyl 2-(1H-tetrazol-1-ylacetyl)hydrazinecarboxylate and benzyl 2-(2H-tetrazol-2-ylacetyl)hydrazine carboxylate in 74% yield.

Step 4: Synthesis of 2-(1H-tetrazol-1-yl)acetohydrazide and 2-(2H-tetrazol-2-yl)acetohydrazide: To a solution of benzyl 2-(1H-tetrazol-1-ylacetyl)hydrazinecarboxylate and benzyl 2-(2H-tetrazol-2-ylacetyl)hydrazinecarboxylate (40 g, 114 mmol, product from Step 3) in methanol (400 ml) was added 10% palladium over carbon (8 g). The solution was hydrogenated at 80 psi at 25-30° C. and progress of reaction was monitored by thin layer chromatography using mixture of chloroform and methanol (9:1). After complete consumption of starting material the reaction mixture was filtered through celite bed and washed with methanol (2×40 ml). The filtrate was concentrated under reduced pressure to get 15 g of mixture of 2-(1H-tetrazol-1-yl)acetohydrazide and 2-(2H-tetrazol-2-yl)acetohydrazide in 73% yield.

Step 5: Synthesis of (2S,5R)-6-(benzyloxy)-7-oxo-N′-(1H-tetrazol-1-ylacetyl)-1,6-diazabicyclo[3.2.1]octane-2-carbohydrazide and 2S,5R)-6-(benzyloxy)-7-oxo-N′-(2H-tetrazol-2-ylacetyl)-1,6-diazabicyclo[3.2.1]octane-2-carbohydrazide: To a suspension of 2-(1H-tetrazol-1-yl)acetohydrazide and 2-(2H-tetrazol-2-yl)acetohydrazide (15 g, 105 mmol, product from Step 4) in water (150 ml) was added sodium (2S,5R)-6-(benzyloxy)-7-oxo-1,6-diazabicyclo[3.2.1]octane-2-carboxylate (31.47 g, 105 mmol) under stirring. To this resulting reaction mass was added EDC.HCl (40.35 g, 211 mmol) followed by hydroxy benzotriazole (14.26 g, 105 mmol) under stirring. The progress of reaction was monitored by thin layer chromatography using mixture of chloroform and methanol (9:1). The white precipitates obtained were filtered and washed with water (2×100 ml) and dried under vacuum to provide 32 g of crude product. This was purified by column silica gel chromatography by using mixture of chloroform and methanol as an eluent. The pure fractions were collected and on concentration provided 10 g of (2S,5R)-6-(benzyloxy)-7-oxo-N′-(1H-tetrazol-1-ylacetyl)-1,6-diazabicyclo[3.2.1]octane-2-carbohydrazide and 13 g of 2S,5R)-6-(benzyloxy)-7-oxo-N-(2H-tetrazol-2-ylacetyl)-1,6-diazabicyclo[3.2.1]octane-2-carbohydrazide in 54% yield.

Step 6: Synthesis of (2S,5R)-6-(hydroxy)-7-oxo-N′-(1H-tetrazol-1-ylacetyl)-1,6-diazabicyclo[3.2.1]octane-2-carbohydrazide:

To a solution of (2S,5R)-6-(benzyloxy)-7-oxo-N-(1H-tetrazol-2-ylacetyl)-1,6-diazabicyclo[3.2.1]octane-2-carbohydrazide (6 g, 1.5 mmol, lower spot product from Step 5) in dichloromethane (30 ml) and N,N′-dimethylformamide (30 ml) containing 10% palladium over carbon (1.8 mg, 50% wet) was hydrogenated, at 55 psi for 2 hours at 25° C. The resulting mixture was filtered through a celite pad. The residue was washed with dichloromethane (60 ml). The solvent from the filtrate was evaporated under reduced pressure to obtain the 4.65 g of the titled product as oil in about 100% yield, which was used as such for the next reaction without further purification.

Step 7: Synthesis of tetrabutyl ammonium salt of (2S,5R)-6-(sulfooxy)-7-oxo-N′-(1H-tetrazol-1-ylacetyl)-1,6-diazabicyclo[3.2.1]octane-2-carbohydrazide: To a stirred solution of (2S,5R)-N′-[(5-tert-butoxycarbonylamino-1H-tetrazol-1-yl)acetyl]-7-oxo-6-oxy-1,6-diazabicyclo[3.2.1]octane-2-carbohydrazide (4.65 g, 1.5 mmol, product from Step 6) in dimethylformamide (30 ml) was added dimethylformamide sulfur trioxide complex (2.76 g, 1.8 mmol) in one portion at 0° C. under argon atmosphere. The reaction mass was stirred at the same temperature for 30 minutes and allowed to attain ambient temperature. The reaction was monitored by thin layer chromatography using mixture of chloroform and methanol (9:1) as solvent. After complete consumption of starting material tetrabutyl ammonium acetate (5.42 g, 1.8 mmol) dissolved in 20 ml of water was added to it at 25-30° C. under stirring. The reaction was monitored by thin layer chromatography using mixture of chloroform and methanol (9:1) as solvent. After complete consumption of starting material the volatiles were removed under reduced pressure. The residue was partitioned between dichloromethane (200 ml) and water (100 ml). The water layer was separated and organic layer washed with water (100 ml). The organic extract was dried over anhydrous sodium sulfate and concentrated under reduced pressure to provide 7.8 g of tetrabutyl ammonium salt of (2S,5R)-6-(sulfooxy)-7-oxo-N′-(1H-tetrazol-1-ylacetyl)-1,6-diazabicycle[3.2.1]octane-2-carbohydrazide in 82% yield.

Analysis:

Mass: 389.3 (M−1) for free acid; for Molecular weight: 631 and Molecular formula: C₁₀H₁₃N₈O₇S. C₁₆H₃₆N;

¹H NMR (400 MHz, DMSO-d₆): δ 7.93 (s, 1H), 5.33 (s, 2H), 3.97 (bs, 1H), 3.83(m, 1H), 3.14-3.10 (m, 9H), 2.87(m, 1H), 2.11-1.5 (m, 4H), 1.54 (m, 8H), 1.33-1.24 (m, 8H), 0.93-0.89 (m, 12H).

Step 8: Synthesis of sodium salt of (2S,5R)-6-(sulfooxy)-7-oxo-N′-(1H-tetrazol-1-ylacetyl)-1,6-diazabicyclo[3.2.1]octane-2-carbohydrazide: A column loaded 315 g Sodium Resin (INDION 225 Na⁺ resin) was eluted with HPLC grade water (1000 ml) followed by 10% tetrahydrofuran in water (1000 ml). Tetrabutylammonium salt of (2S,5R)-6-(sulfoxy)-7-oxo-N′-(1H-tetrazol-1-ylacetyl)-1,6-diazabicycle[3.2.1]octane-2-carbohydrazide (7.8 g, 1.2 mmol, product from Step 7) dissolved in tetrahydrofuran (10 ml) was charged on column. The column was eluted with 10% tetrahydrofuran in water and fractions of 50 ml were collected. The fractions containing product were confirmed by performing thin layer chromatography using mixture of chloroform and methanol as solvent system. The collected required fractions were concentrated under reduced pressure to remove tetrahydrofuran. The aqueous eluent was washed with dichloromethane (2×100 ml) and concentrated under reduced pressure to provide 3.6 g of sodium salt of (2S,5R)-6-sulfoxy-N′-[(5-ethoxycarbonyl)-1H-tetrazol-1-yl)acetyl]-7-oxo-1,6-diazabicyclo[3.2.1]octane-2-carbohydrazide in 70% yield.

Analysis:

Melting Point: 143-145° C. (decomposes);

Mass: 389.2 (M−1), 391.2 (M+1) for free acid, for Molecular weight: 412 and Molecular formula: C₁₀H₁₂N₈O₇S.Na;

¹H NMR (400 MHz, DMSO-d₆-D₂O): δ 9.39 (s, 1H), 5.37(s, 2H), 3.99 (s, 1H), 3.85 (m, 1H), 3.11 (m, 1H), 3.00 (m, 1H), 2.06-2.00 (m, 1H), 1.87-1.59 (m, 3H); Purity as determined by HPLC: 97.97%.

The compounds of Examples 4 to 23 (Table 1) were prepared using the procedure described in Example 3 and corresponding starting reagents in place of 2-(1H-tetrazol-1-yl)acetohydrazide or 2-(2H-tetrazol-1-yl)acetohydrazide. The isomeric mixtures were separated at benzyl stage product (Step 5) to single isomers using silica gel column chromatography or else further followed as per the procedures described in Example 3.

TABLE 1

| Example | | | | Mass |
|---|---|---|---|---|
| No. | Compound | Starting Reagent | ¹HNMR (400 MHz) | (As free acid) |
| 4 | 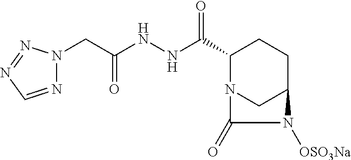 | 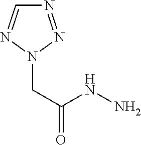 | (DMSO-d₆-D₂O exchange): δ 9.39 (s, 1H), 5.37 (s, 2H), 3.99 (s, 1H), 3.85 (m, 1H), 3.11 (m, 1H), 3.00 (m, 1H), 2.06-2.00 (m, 1H), 1.87-1.59 (m, 3H) | 391.2 [M + 1] (C₁₀H₁₂N₈O₇SNa) |
| 5 | 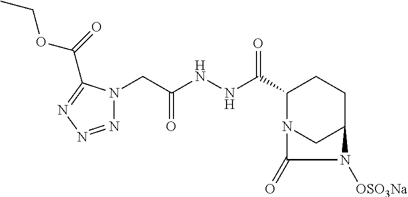 | 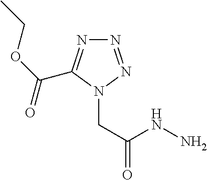 | (DMSO-d₆-D₂O exchange): δ 5.73 (s, 2H), 4.46 (q, 2H, J = 6.8 Hz), 3.99 (s, 1H), 3.86 (m, 1H), 3.13 (m, 1H), 3.01 (m, 1H), 2.06 (m, 1H), 1.88- 1.59 (m, 3H), 1.59 (t, 3H, J = 7.2 Hz) | 463.3 [M + 1] (C₁₃H₁₇N₈O₉SNa) |
| 6 | 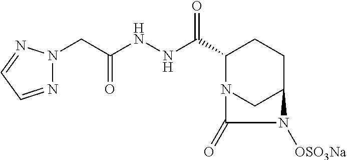 | 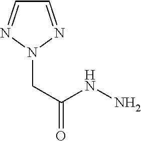 | (DMSO-d₆): δ 10.25 (1H, s), 10.06 (1H, s), 7.81 (2H, d, J = 6 Hz), 5.21 (2H, s), 3.98 (1H, s), 3.83 (1H, d, J = 7.2 Hz), 3.17 (1H, J = 12 Hz), 2.97 (1H, d, J = 11.6 Hz), 2.03-1.99 (1H, m), 1.85-1.59 (3H, m) | 390.2 [M + 1] (C₁₁H₁₄N₇O₇SNa) |
| 7 | 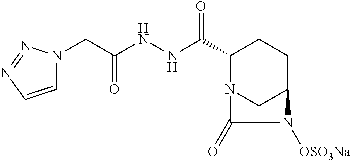 | 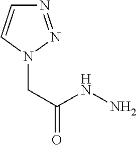 | (DMSO-d₆): δ 10.25 (1H, s), 8.10 (1H, s), 7.73 (1H, s), 5.21 (2H, s), 3.99 (1H, s), 3.84 (1H, d, J = 7.6 Hz), 3.25 (1H, J = 12 Hz), 2.98 (1H, d, J = 11.6 Hz), 2.05-2.0 (1H, m), 1.87-1.59 (3H, m). | 390.2 [M + 1] (C₁₁H₁₄N₇O₇SNa) |
| 8 | 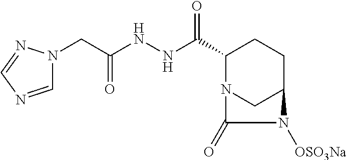 | 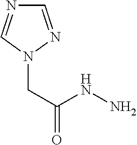 | (DMSO-d₆): δ 10.20 (br s, 2H), 8.48 (s, 1H), 7.94 (s, 1H), 5.00 (2H, s), 3.99 (1H, s), 3.83 (1H, d, J = 7.6 Hz), 3.17 (1H, J = 12 Hz), 2.98 (1H, d, J = 11.2 Hz), 2.05-2.0 (1H, m), 1.87-1.59 (3H, m). | 390.2 [M + 1] (C₁₁H₁₄N₇O₇SNa) |
| 9 | 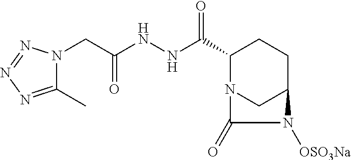 | 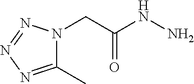 | (D₂O): δ 5.33 (s, 2H), 4.10 (bs, 2H), 3.24 (s, 1H), 3.08 (m, 1H), 2.48 (s, 3H), 2.13 (m, 1H), 2.00 (m, 1H), 1.88-1.83 (m, 1H), 1.73 (m, 1H) | 405.3 [M + 1] (C₁₁H₁₅N₈O₇SNa) |
| 10 | 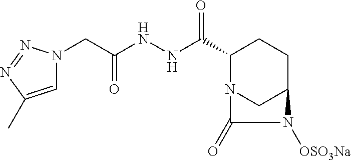 | 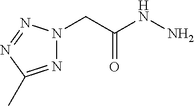 | (D₂O): δ 5.52 (s, 2H), 4.09 (bs, 2H), 3.24 (s, 1H), 3.08 (m, 1H), 2.42 (s, 3H), 2.11 (m, 1H), 1.94- 1.70 (m, 3H) | 405.3 [M + 1] (C₁₁H₁₅N₈O₇SNa) |
| 11 | 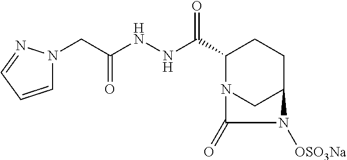 | 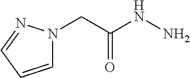 | (DMSO-d₆): δ 10.1 (2H, br s), 7.70 (1H, d, J = 2 Hz), 7.41 (1H, d, J = 1.6 Hz), 6.23 (1H, t, J = 2 Hz, 2 Hz), 4.82 (2H, s), 3.97 (1H, s), 3.80 (1H, d, J = 6.8 Hz), 3.58 (1H, t, J = 2.4, 2.4 Hz), 3.15 (1H, d, J = 12 Hz), 2.95 (1H, d, J = 12 Hz), 2.04-1.99 (1H, m), 1.86-1.82 (1H, m), 1.75-1.57 (2H, m) | 389.3 [M + 1] (C₁₂H₁₅N₆O₇Sna) |
| 12 | 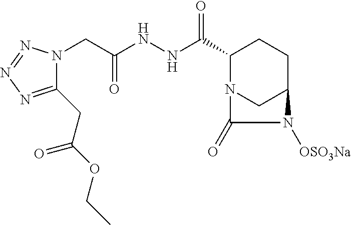 | 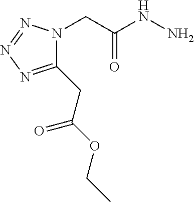 | (DMSO-d₆ D₂O exchange): δ 5.32 (s, 2H), 4.20 (s, 2H), 4.15-4.07 (m, 2H), 3.96 (s, 1H), 3.83 (d, 1H), 3.10 (m, 1H), 2.96 (m, 1H), 2.03-1.56 (m, 4H), 1.18-1.15 (m, 3H) | 475.4 [M − 1] 477.3 [M + 1] (C₁₄H₁₉N₈O₉SNa) |
| 13 | 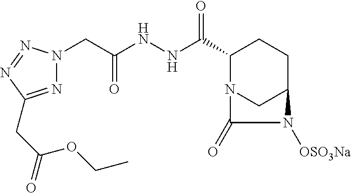 | 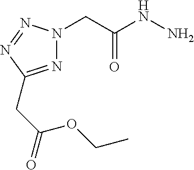 | (DMSO-d₆ D₂O exchange): δ 5.54 (s, 2H), 4.13- 4.08 (m, 2H), 4.04 (s, 2H), 3.96 (s, 1H), 3.83 (d, 1H), 3.12 (m, 1H), 2.97 (m, 1H), 2.02-1.68 (m, 4H), 1.19-1.15 (m, 3H) | 475.3 [M − 1] 477.3 [M + 1] (C₁₄H₁₉N₈O₉SNa) |
| 14 | 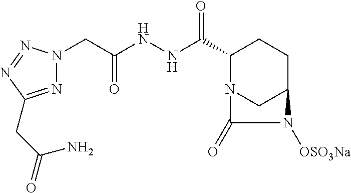 | 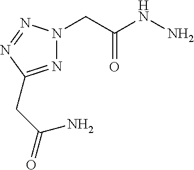 | (DMSO-d₆ D₂O exchange): δ 5.50 (s, 2H), 3.95 (s, 1H), 3.82 (d, 1H), 3.71 (s, 2H) 3.11 (m, 1H), 2.98 (m, 1H), 2.01-1.56 (m, 4H) | 446.2 [M − 1] 448.2 [M + 1] (C₁₂H₁₆N₉O₈SNa) |
| 15 | 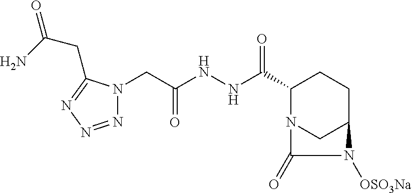 | 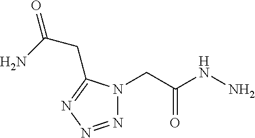 | (DMSO-d₆ D₂O exchange): δ 5.36 (s, 2H), 3.98 (m, 1H), 3.91 (s, 2H), 3.84 (m, 1H), 3.09-2.99 (m, 2H), 2.04-1.60 (m, 4H). | 446.0 [M − 1] 448.0 [M + 1] (C₁₂H₁₆N₉O₈SNa) |
| 16 | 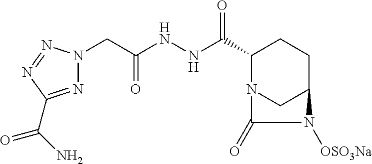 | 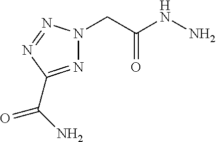 | (DMSO-d₆ D₂O exchange): δ 5.56 (s, 2H), 3.98 (s, 1H), 3.82 (d, 1H), 3.10 (m, 1H), 2.99 (m, 1H), 2.05-1.59 (m, 4H) | 432.3 [M − 1] 434.3 [M + 1] (C₁₁H₁₄N₉O₈SNa) |
| 17 | 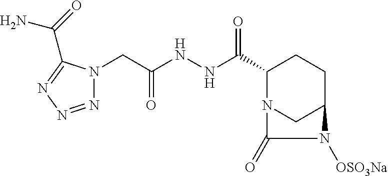 | 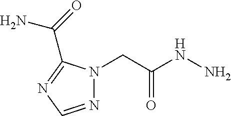 | (DMSO-d₆ D₂O exchange): δ 5.69 (s, 2H), 3.99 (m, 1H), 3.87 (s, 1H), 3.15 (m, 1H), 3.01 (m, 1H), 2.04-1.60 (m, 4H). | 432.2 [M − 1] (C₁₁H₁₄N₉O₈ SNa) |
| 18 | 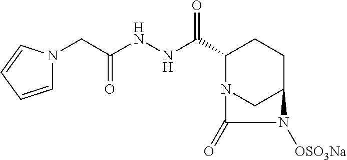 | 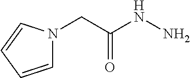 | (DMSO-d₆ D₂O exchange): δ 6,72 (s, 2H), 6.00 (s, 2H), 4.59 (s, 2H), 3.99 (m, 1H), 3.84 (m, 1H), 3.22 (m, 1H), 3.00 (m, 1H), 2.05-1.60 (m, 4H). | 386.1 [M − 1] 388.1 [M + 1] (C₁₃H₁₆N₅O₇SNa) |
| 19 | 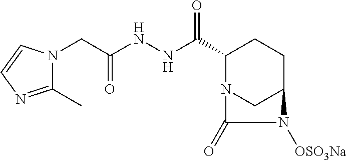 | 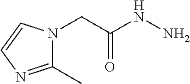 | (DMSO-d₆): δ 10.09 (s, 2H), 6.92 (s, 1H), 6.68 (s, 1H), 4.63 (s, 2H), 3.99 (s, 1H), 3.81 (d, 1H, J = 6.8 Hz), 3.16 (d, 1H, J = 12 Hz), 3.00 (d, 1H, J = 11.6), 2.23 (s, 3H), 2.11-1.59 (m, 4H) | 401.2 [M − 1] (C₁₃H₁₇N₆O₇S•Na) |
| 20 | 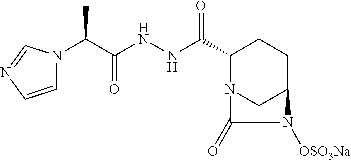 | 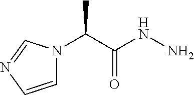 | (DMSO-d₆): δ 10.12 (brs, 2H), 7.68 (s, 1H), 7.20 (s, 1H), 6.86 (s, 1H), 4.91 (q, 1H, J = 7.6 Hz), 3.97 (brs, 1H), 3.80 (d, 1H, J = 6.8 Hz), 3.14 (d, 1H, J = 12.0 Hz), 2.98 (d, 1H, J = 12.0 Hz), 2.06-1.96 (m, 1H), 1.88-1.58 (m, 3H), 1.56 (d, 3H, J = 7.2 Hz) | 401.2 [M − 1] (C₁₃H₁₇N₆O₇S•Na) |
| 21 | 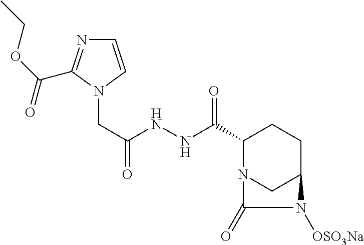 | 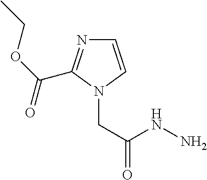 | (DMSO-d₆): δ 10.11 (brs, 2H), 7.47 (s, 1H), 7.08 (s, 1H), 5.13 (s, 2H), 4.29 (q, 2H, J = 8 Hz), 3.98 (s, 1H), 3.83 (d, 1H, J = 8 Hz), 3.19-3.16 (d, 1H, J = 12 Hz), 2.98-2.95 (d, 1H, J = 12 Hz), 2.02-1.59 (m, 4H), 1.27 (t, 3H, J = 8 Hz). | 459.2 [M − 1] 461.2 [M + 1] (C₁₅H₁₉N₆O₉S•Na) |
| 22 | 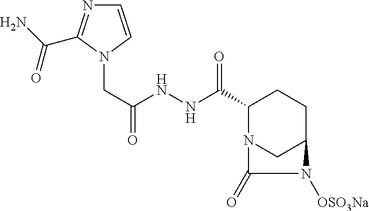 | 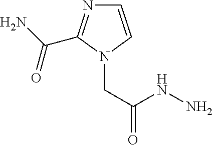 | (DMSO-d₆): δ 9.98 (bs, 2H), 7.54 (bs, 1H), 7.35-7.34 (d, 1H, J = 6 Hz), 7.12 (bs, 1H), 7.02 (s, 1H), 5.07-5.06 (d, 2H, J = 6 Hz), 4.02 (s, 1H), 3.83- 3.81 (d, 1H, J = 7 Hz), 3.27-3.24 (d, 1H, J = 12 Hz), 3.04-3.01 (d, 1H, J = 12 Hz), 2.03 (m, 1H), 1.86 (m, 1H), 1.70 (m, 2H). | 430.3 [M − 1] 432.3 [M + 1] (C₁₃H₁₆N₇O₈S•Na) |
| 23 | 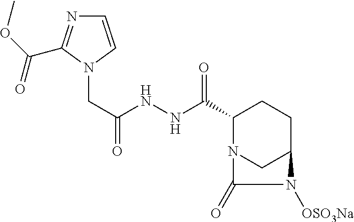 | 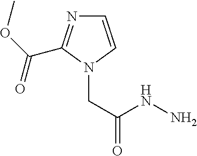 | ¹H NMR: (DMSO-d₆ D₂O exchange): δ 10.00 (bs, 2H), 7.44 (s, 1H), 7.08 (s, 1H), 5.27 (s, 2H), 4.02 (s, 1H), 3.83-3.81 (d, 1H, J = 7 Hz), 3.67 (s, 3H), 3.24-3.21 (d, 1H, J = 12 Hz), 3.04-3.01 (d, 1H, J = 12 Hz), 2.06-2.01 (m, 2H), 1.76-1.64 (m, 2H). | 445.3 [M − 1] 447.3 [M + 1] (C₁₄H₁₇N6O₉S•Na) |

### Example 24

### Synthesis of (2S,5R)-N′-[(5-amino-1H-tetrazol-1-yl)acetyl]-7-oxo-6-(sulfooxy)-1,6-diazabicyclo[3.2.1]octane-2-carbohydrazide

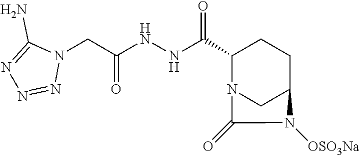

Step 1: Synthesis of ethyl {5-[(tert-butoxycarbonyl)amino]-1H-tetrazol-1-yl}acetate: To a solution of ethyl (5-amino-1H-tetrazol-1-yl)acetate (10 g, 58.4 mmol, prepared according to the procedure described in European Journal of Organic Chemistry, 2013(4), 688-692; 2013) in dichloromethane (100 ml) was added triethyl amine (16.5 ml, 116.9 mmol) at 25-30° C. To this reaction mass was added Boc anhydride (12.74 g, 58.4 mmol) followed by dimethyl amino pyridine (72 mg, 5 mmol) under stirring. The progress of the reaction was monitored by performing thin layer chromatography using mixture of chloroform and methanol (9:1) as solvent. After complete consumption of starting material, water (100 ml) was added to the reaction mixture. The organic extract was collected and dried over anhydrous sodium sulfate and then concentrated to provide 6.3 g of ethyl15-[(tert-butoxycarbonyl)amino]-1H-tetrazol-1-yl}acetate in 40% yield.

Step 2: Synthesis of tert-butyl [1-(2-hydrazinyl-2-oxoethyl)-1H-tetrazol-5-yl]carbamate: To a solution of ethyl {5-[(tert-butoxycarbonyl)amino]-1H-tetrazol-1-yl}acetate (6.3 g, 23.2 mmol, product from Step 1) in ethanol (60 ml) was added hydrazine hydrate (1.16 g, 23.2 mmol) under stirring at 25-30° C. The progress of the reaction was monitored by thin layer chromatography using mixture of chloroform and methanol (9:1) as solvent. After complete consumption of starting material, the volatiles were removed under reduced pressure to provide a yellowish residue. To this was added diethyl ether (100 ml) and stirred to obtain the precipitates of the required compound. The obtained precipitates were filtered and washed with ether (2×25 ml) to provide 6 g of tert-butyl [1-(2-hydrazinyl-2-oxoethyl)-1H-tetrazol-5-yl]carbamate in 100% yield.

Step 3. (2S,5R)-N′-[(5-tert-butylamino-1H-tetrazol-1-yl)acetyl]-6-(benzyloxy)-7-oxo-1,6-diazabicyclo[3.2.1]octane-2-carbohydrazide: To a solution of sodium (2S,5R)-6-(benzyloxy)-7-oxo-1,6-diazabicyclo[3.2.1]octane-2-carboxylate (6.95 g, 23 mmol) in dimethylformamide (25 ml) was added HATU (8.87 g, 23 mmol) at 25-30° C. To this was added diisopropyl ethylamine (8 ml, 46 mmol) followed by tert-butyl [1-(2-hydrazinyl-2-oxoethyl)-1H-tetrazol-5-yl]carbamate (6 g, 23 mmol, product from Step 2) under stirring. The progress of the reaction was monitored by performing thin layer chromatography using mixture of chloroform and methanol (9:1). After complete consumption of the starting material the reaction mixture was added to water (600 ml) and extracted with ethyl acetate (2×200 ml). The organic extracts were combined and washed with 5% potassium hydrogen sulfate solution (1×100 ml), saturated sodium hydrogen carbonate solution (1×100 ml) and brine (1×100 ml). The volatiles were removed under reduced pressure to get 8 g of crude product. The product was purified by column chromatography using silica gel 100-200 mesh size using mixture of chloroform and methanol as an eluent. The fractions containing the required compound were collected and concentrated to obtain 3.3 g of the titled product in 28% yield.

Step 4: Synthesis of (2S,5R)-N′-[(5-tert-butoxycarbonylamino-1H-tetrazol-1-yl)acetyl]-7-oxo-6-hydroxy-1,6-diazabicyclo[3.2.1]octane-2-carbohydrazide: To a solution of (2S,5R)-N′-[(5-tert-butoxycarbonylamino-1H-tetrazol-1-yl)acetyl]-6-(benzyloxy)-7-oxo-1,6-diazabicyclo[3.2.1]octane-2-carbohydrazide (1.4 g, 2.7 mmol, product from Step 3) in dichloromethane (7 ml) and N,N′ dimethyl formamide (7 ml) was added 10% palladium over carbon (420 mg, 50% wet) under 55 psi hydrogen pressure and stirred for 2 hours at 25° C. The resulting mixture was filtered through a celite pad. The residue was washed with dichloromethane (40 ml). The solvent from the filtrate was evaporated under reduced pressure to provide 1.15 g of the titled product as oil in 100% yield, which was used as such in the next step without further purification.

Step 5: Synthesis of tetrabutyl ammonium salt of (2S,5R)-N′-[(5-tert-butoxycarbonylamino-1H-tetrazol-1-yl)acetyl]-7-oxo-6-(sulfooxy)-1,6-diazabicyclo[3.2.1]octane-2-carbohydrazide: To a stirred solution of (2S,5R)-N′-[(5-tert-butoxycarbonylamino-1H-tetrazol-1-yl)acetyl]-7-oxo-6-hydroxy-1,6-diazabicyclo[3.2.1]octane-2-carbohydrazide (1.15 g, 2.7 moles, product from Step 4) in dimethylformamide (14 ml) was added dimethyl sulfur trioxide complex (500 mg, 3.2 mmol) in one portion under stirring in presence of argon atmosphere at 0° C. The reaction mass was stirred at the same temperature for 30 minutes and allowed to attain ambient temperature. The progress of reaction was monitored by performing thin layer chromatography using mixture of chloroform and methanol (9:1) as solvent. After complete consumption of the starting material tetrabutyl ammonium acetate (1 g, 3.2 mmol) dissolved in water (3 ml) was added to the reaction mixture under stirring at 25-30° C. The reaction was monitored by thin layer chromatography using mixture of chloroform and methanol (9:1). After complete consumption of starting material the volatiles were removed under reduced pressure. The residue obtained was partitioned between dichloromethane (140 ml) and water (140 ml). The water layer was separated and organic layer washed with water (70 ml). The collective organic extract was dried and concentrated under reduced pressure to provide 1.45 g of the titled product as off-white foam in 72% yield.

Analysis:

Mass spectrum: 504.3 (M−1), 506.2 (M+1) for free acid, for Molecular weight: 746 and for Molecular formula: C₁₅H₂₂N₉O₉S, C₁₆H₃₆N;

¹HNMR (400 MHz, DMSO-d₆): δ 5.16 (s, 2H), 3.98 (bs, 1H), 3.83 (m, 1H), 3.18-3.10 (m, 9H), 2.99 (m, 1H), 2.02-1.65 (m, 4H), 1.58 (m, 8H), 1.46 (s, 9H), 1.35-1.26 (m, 8H), 0.95-0.91 (m, 12H).

Step 6: Synthesis of (2S,5R)-N′-[(5-amino-1H-tetrazol-1-yl)acetyl]-7-oxo-6-(sulfooxy)-1,6-diazabicyclo[3.2.1]octane-2-carbohydrazide: To a solution of tetrabutyl ammonium salt of (2S,5R)-N′-[(5-tert-butoxycarbonylamino-1H-tetrazol-1-yl)acetyl]-7-oxo-6-(sulfooxy)-1,6-diazabicyclo[3.2.1]octane-2-carbohydrazide (250 mg, 0.335 mmol, product obtained from Step 5) in dichloromethane (1.25 ml) added trifluoroacetic acid (1.25 ml) drop wise at 0° C. over a period of 5 minutes under stirring in argon atmosphere. The resulting mass was further stirred at same temperature for 1 hr. The progress of the reaction was monitored by Mass. After complete consumption of starting material the resulting mixture was concentrated under reduced pressure to get an oily residue. To this resulting mass was added acetonitrile (20 ml) and concentrated under reduced pressure to remove traces of trifluoroacetic acid. The resulting oily residue was stirred in diethyl ether (20 ml) for 30 minutes to obtain precipitates of product. The ether layer was removed by decantation from the precipitated solid. This procedure was repeated twice again with diethyl ether (2×20 ml). The solid thus obtained was filtered and washed with diethyl ether (2×25 ml). The solid was dried at 25° C. under reduced pressure to provide 60 mg of the titled product as off-white solid in 44% yield.

Analysis:

Mass: 418.1 (M−1), 420.2 (M+1) for free acid, for Molecular weight: 419 and Molecular formula: C₁₁H₁₇N₉O₇S.

¹HNMR (400MHz, DMSO-d₆-D₂O exchange): δ 5.45 (s, 2H), 4.48 (s, 2H), 4.00 (s, 1H), 3.87 (m, 1H), 3.11 (m, 1H), 3.01 (m, 1H), 2.03 (m, 1H), 1.85-1.56 (m, 3H).

The compounds of Examples 25 to 37 (Table 2) were prepared using the procedure described in Example 24 and corresponding starting reagents in place of tert-butyl [1-(2-hydrazinyl-2-oxoethyl)-1H-tetrazol-5-yl]carbamate.

TABLE 2

| Ex- | | | | |
|---|---|---|---|---|
| am- | | | | |
| ple | | | | Mass |
| No. | Compound | Starting Reagent | ¹HNMR (400 MHz) | (As free acid) |
| 25 | 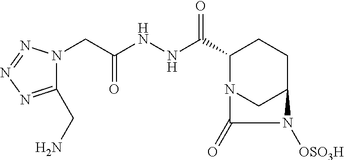 | 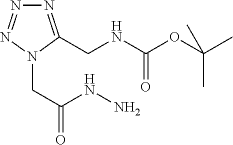 [SMILES: CC(C)(C)OC(=O)NCc1nnnn1CC(=O)NN] | (DMSO-d₆-D₂O exchange): δ 5.45 (s, 2H), 4.48 (s, 2H), 4.00 (s, 1H), 3.87 (m, 1H), 3.11 (m, 1H), 3.01 (m, 1H), 2.03 (m, 1H), 1.85-1.56 (m, 3H) | 420.2 [M − 1] (C₁₁H₁₇N₉O₇S) |
| 26 | 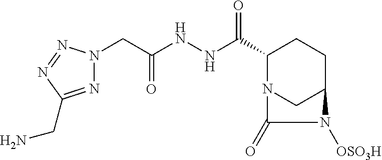 | 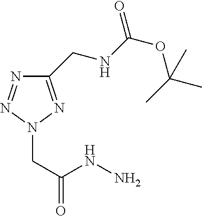 [SMILES: CC(C)(C)OC(=O)NCc1nnn(CC(=O)NN)n1] | (DMSO-d₆-D₂O exchange): δ 5.63 (s, 2H), 4.43 (s, 2H), 4.01 (s, 1H), 3.87 (m, 1H), 3.14 (m, 1H), 3.02 (m, 1H), 2.03 (m, 1H), 1.86-1.55 (m, 3H) | 420.2 [M − 1] (C₁₀H₁₅N₉O₇S) |
| 27 | 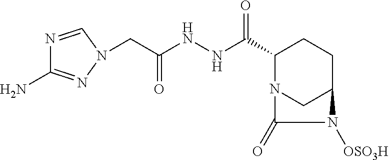 | 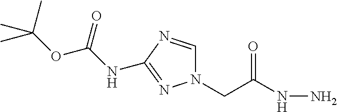 [SMILES: CC(C)(C)OC(=O)Nc1ncn(CC(=O)NN)n1] | (DMSO-d₆): δ 10.26 (s, 1H), 10.10 (s, 1H), 8.29 (s, 1H), 8.18 (s, 2H), ), 4.85- 4.75 (2H, m), 3.99 (1H, s), 3.83 (1H, d, J = 6.8 Hz), 3.14 (1H, J = 12 Hz), 2.97 (1H, d, J = 11.6 Hz), 2.02-1.84 (2H, m), 1.73-1.67 (2H, m). | 403.3 [M − 1] (C₁₁H₁₆N₈O₇S) |
| 28 | 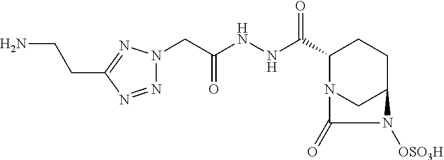 | 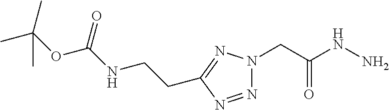 | (DMSO-d₆ D₂O exchange): δ 5.56 (s, 2H), 4.00 (m, 1H), 3.87 (m, 1H), 3.27-3.18 (m, 4H), 3.14-3.11 (m, 1H), 3.02-3.00 (m, 1H), 2.06-1.60 (m, 4H) | 432.3 [M − 1] 434.1 [M + 1] (C₁₂H₁₉N₉O₇S) |
| 29 | 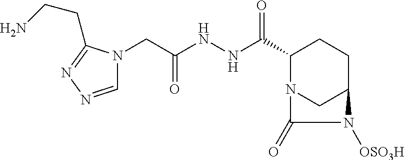 | 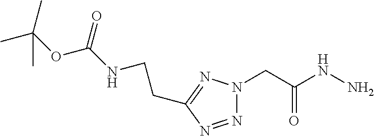 | (DMSO-d₆ D₂O exchange): δ 5.33 (s, 2H), 4.02 (m, 1H), 3.89 (m, 1H), 3.27-3.20 (m, 4H), 3.11-3.08 (m, 2H), 2.06-1.62 (m, 4H) | 432.2 [M − 1]] 434.1 [M + 1] (C₁₂H₁₉H₉O₇S) |
| 30 | 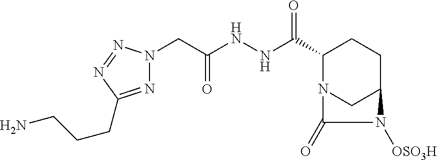 | 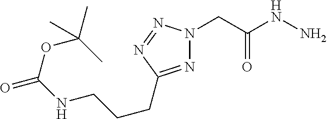 | (DMSO-d₆ D₂O exchange): δ 5.52 (s, 2H), 4.01 (m, 1H), 3.87 (m, 1H), 3.14-2.90 (m, 6H), 2.06-1.97 (m, 2H), 1.87-1.60 (m, 4H). | 446.4 [M − 1] 448.3 [M + 1] (C₁₃H₂₁N₉O₇S) |
| 31 | 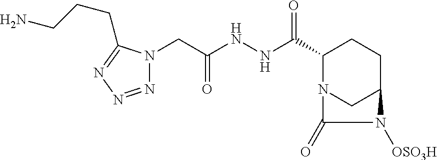 | 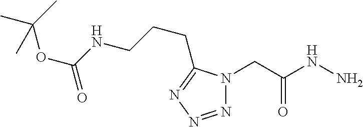 | (DMSO-d₆ D₂O exchange): δ 5.28 (s, 2H), 4.01 (m, 1H), 3.87 (m, 1H), 3.12-2.89 (m, 6H), 2.08-2.02 (m, 2H), 1.86-1.77 (m, 4H). | 446.4 [M − 1] 448.3 [M + 1] (C₁₃H₂₁N₉O₇S) |
| 32 | 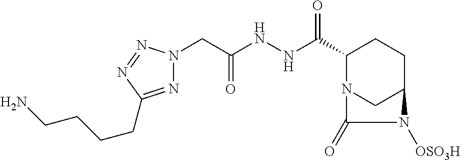 | 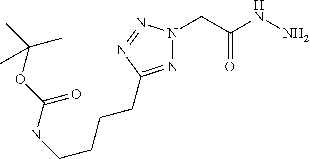 | (DMSO-d₆ D₂O exchange): δ 5.45 (s, 2H), 3.98 (m, 1H), 3.85 (m, 1H), 3.11-2.98 (m, 4H), 2.88-2.78 (m, 4H), 2.04-1.99 (m, 2H), 1.87-1.59 (m, 6H). | 460.4 [M − 1] 462.4 [M + 1] (C₁₄H₂₃N₉O₇S) |
| 33 | 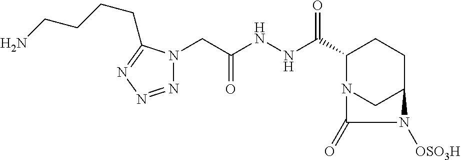 | 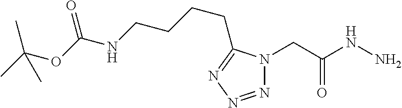 | (DMSO-d₆ D₂O exchange): δ 5.27 (s, 2H), 3.99 (m, 1H), 3.85 (m, 1H), 3.11-2.99 (m, 4H), 2.89-2.79 (m, 4H), 2.03-1.84 (m, 2H), 1.81.58 (m, 6H). | 460.4 [M − 1] 462.3 [M + 1] (C₁₄H₂₃N₉O₇S) |
| 34 | 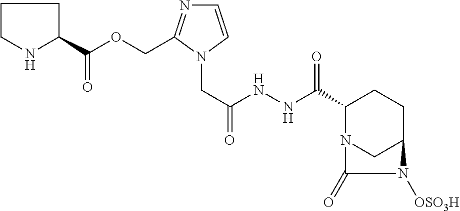 | 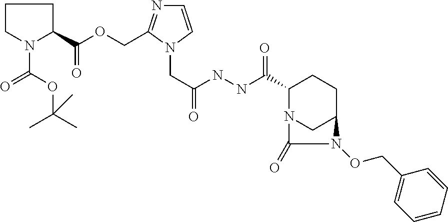 | (DMSO-d₆): δ 10.3 (bs, 2H), 10.14 (s, 2H), 7.52 (s, 1H), 7.35 (s, 1H), 5.39 (s, 2H), 5.01 (s, 2H), 4.42 (t, 1H, J = 9.7 Hz), 4.01 (s, 1H), 3.88-3.87 (d, 1H, J = 7 Hz), 3.22 (m, 2H), 3.15-3.12 (d, 1H, J = 12 Hz), 2.96-2.67 (d, 1H, J = 12 Hz), 2.33 (m, 1H), 2.24 (m, 1H), 2.01 (m, 2H), 1.94 (m, 2H), 1.72 (m, 2H). | 514.3 [M − 1] 516.4 [M + 1] (C₁₈H₂₅N₇O₉S) |
| 35 | 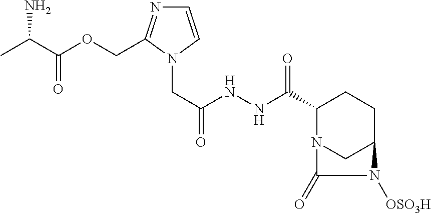 | 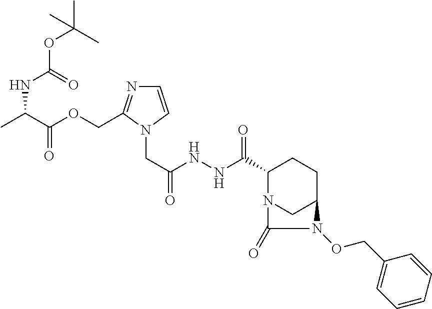 | (DMSO-d₆): δ 10.38 (bs, 1H), 10.12 (s, 1H), 8.29 (bs, 3H), 7.47 (s, 1H), 7.27 (s, 1H), 5.37 (s, 2H), 4.93 (s, 2H), 4.13 (s, 1H), 4.01 (s, 1H), 3.88-3.86 (d, 1H, J = 8 Hz), 3.15-3.12 (d, 1H, J = 12 Hz), 3.02-2.99 (d, 1H, J = 12 Hz), 2.06-1.60 (m, 4H), 1.40-1.38 (d, 3H, J = 6 Hz). | 488.4 [M − 1] 490.5 [M + 1] (C₁₆H₂₃N₇O₉S) |
| 36 | 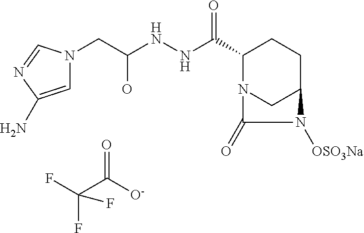 | 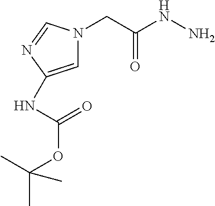 | (DMSO-d₆): δ 10.26 (s, 1H), 10.09 (s, 1H), 8.09 (s, 2H), 6.46 (s, 2H), 4.79 (s, 2H), 4.00 (s, 1H), 3.84-3.86 (d, 1H), 3.16-3.19 (d, 1H), 2.98-3.01 (d, 1H), 1.60-2.02 (m, 4H). | 402.2 [M − 1] 404.2 [M + 1] (C₁₂H₁₆N₇O₇S•C₂HO₂F₃Na) |
| 37 | 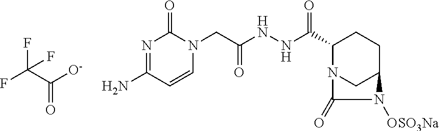 | 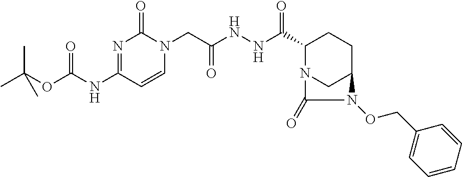 [SMILES: CC(C)(C)OC(=O)Nc1ccn(CC(=O)NNC(=O)[C@@H]2CC[C@@H]3CN2C(=O)N3OCc2ccccc2)c(=O)n1] | (DMSO-d₆): δ 10.20 (s, 1H), 10.05 (s, 1H), 8.92 (s, 1H), 8.08 (s, 1H), 7.84-7.88 (d, 1H), 5.96-5,98 (d, 1H), 4.51 (s, 2H), 4.00-4.03 (d, 1H), 3.82-3.84 (d, 1H), 3.19-3.20 (d, 1H), 2.97-3.00 (d, 1H), 1.60-2.00 (m, 4H). | 430.2 [M − 1] (C₁₃H₁₆N₇O₈S•C₂F₃O₂•Na) |

### Example 38

### Synthesis of Sodium salt of (2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide

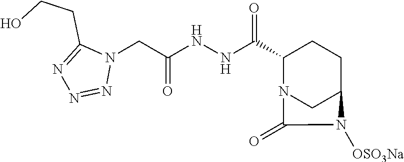

Step 1: Synthesis of (2S,5R)-6-benzyloxy-N′-{[5-(2-tert-butyldimethysilanyloxyethyl)-1H-tetrazol-1-yl]acetyl}-7-oxo-1,6-diazabicyclo[3.2.1]octane-2-carbohydrazide: To a clean dry flask containing solution of {5-[2-(tert-butyl-dimethyl-silanyloxy)-ethyl]-tetrazol-1-yl}-acetic acid hydrazide (22 g, 73 mmol, prepared according to the procedure described in Preparation-33) in dimethylformamide (220 ml) was added sodium salt of (2S,5R)-6-(benzyloxy)-7-oxo-1,6-diazabicyclo[3.2.1]octane-2-carboxylic acid (23 g, 73 mmol) under stirring. To resulted reaction mass was added EDC.HCl (21 g, 109 mmol) followed by HOBt.H₂O (11.21 g, 73 mmol) and N-methyl morpholine (25 ml, 219 mmol). The progress of reaction was monitored by TLC. After completion of reaction it was poured on to water (2.2 L) under stirring. The product was extracted by ethyl acetate (3×500 ml). The combined organic extract was washed with 1M KHSO₄ solution (250 ml) followed by NaHCO₃ (250 ml) and brine (250 ml). The organic extract thus obtained was dried over anhydrous sodium sulfate and concentrated to get 25 g of crude product. The crude was further purified by column chromatography (silica gel 100-200 mesh size) using chloroform: methanol as an eluent. The pure product obtained at 4% methanol in chloroform was combined and on concentration yielded 18 g of (2S,5R)-6-benzyloxy-N′-{[5-(2-tert-butyldimethysilanyloxyethyl)-1H-tetrazol-1-yl]acetyl}-7-oxo-1,6-diazabicyclo[3.2.1]octane-2-carbohydrazide as off-white foam solid in 44% yield.

Step 2: Synthesis of Tetrabutylammonium salt of (2S,5R)-N′-{[5-(2-tert-butyldimethysilanyloxyethyl)-1H-tetrazol-1-yl]acetyl}-7-oxo-6-sulfooxy-1,6-diazabicyclo [3.2.1]octane-2-carbohydrazide: To a solution of (2S,5R)-6-benzyloxy-N′-{[5-(2-tert-butyldimethysilanyloxyethyl)-1H-tetrazol-1-yl]acetyl}-7-oxo-1,6-diazabicyclo[3.2.1]octane-2-carbohydrazide (8 g, 14.3 mmol) in dimethylformamide (40 ml) and dichloromethane (40 ml) was added 10% Pd/C(50% wet basis) 2.4 g at 25-30° C. The H₂ gas was bubbled through the reaction mixture under stirring. The progress of reaction was monitored by TLC (chloroform: methanol, 9:1). The catalyst was removed by filtration on celite bed and washed with mixture of dichloromethane and dimethylformamide (1:1, 2×20 ml). The filtrate was concentrated under reduced pressure yielded (2S,5R)-6-hydoxy-N′-{[5-(2-tert-butyldimethysilanyloxyethyl)-1H-tetrazol-1-yl]acetyl}-7-oxo-1,6-diazabicyclo[3.2.1]octane-2-carbohydrazide (6.6 g, c.a 100% yield used for next reaction as such). The product (2S,5R)-6-hydoxy-N′-{[5-(2-tert-butyldimethysilanyloxyethyl)-1H-tetrazol-1-yl]acetyl}-7-oxo-1,6-diazabicyclo[3.2.1]octane-2-carbohydrazide (6.6 g, 14.3 mmol) thus obtained was dissolved in dimethylformamide (40 ml) was added dimethylformamide sulfur trioxide complex (2.63 g, 17.20 mmol) in argon atmosphere at 0° C. under stirring. The progress of reaction was monitored by TLC (chloroform: methanol, 9:1). After completion of the reaction added a solution of tetra-butyl ammonium acetate (5.18 g, 17.20 mmol) dissolved in water (18 ml) at 25-30° C. The reaction mixture was stirred for 3 hours and concentrated under reduced pressure. The residue obtained was taken in dichloromethane (80 ml) and washed with water (2×40 ml). The organic extract was dried on anhydrous sodium sulfate and concentrated to yield crude tetrabutylammonium salt of (2S,5R)-N′-{[5-(2-tert-butyldimethysilanyloxyethyl)-1H-tetrazol-1-yl]acetyl}-7-oxo-6-sulfooxy-1,6-diazabicyclo[3.2.1]octane-2-carbohydrazide. This material was purified by column chromatography (silica gel 100-200 mesh size) using chloroform: methanol as an eluent. The fractions containing the product obtained at 5% methanol in chloroform. The pure fractions were combined and concentrated to get 7.5 g of tetrabutylammonium salt of (2S,5R)-N′-{[5-(2-tert-butyldimethysilanyloxyethyl)-1H-tetrazol-1-yl]acetyl}-7-oxo-6-sulfooxy-1,6-diazabicyclo[3.2.1]octane-2-carbohydrazide as off-white foam solid in 66% yield.

Step 3: Synthesis of Tetrabutylammonium salt of (2S,5R)-N′-{[5-(hydroxyethyl)-1H-tetrazol-1-yl]acetyl}-7-oxo-6-sulfooxy-1,6-diazabicyclo[3.2.1]octane-2-carbohydrazide: To a stirred solution of tetrabutylammonium salt of (2S,5R)-N′-{[5-(2-tert-butyldimethysilanyloxyethyl)-1H-tetrazol-1-yl]acetyl}-7-oxo-6-sulfooxy-1,6-diazabicyclo[3.2.1]octane-2-carbohydrazide (7.5 g, 9.4 mmol) in tetrahydrofuran (75 ml) was added tetrabutyl ammonium fluoride (14.2 ml, 14.2 mmol) at 0° C. and then allowed to stir at ambient temperature. The progress of reaction was monitored by TLC (dichloromethane: methanol, 9:1), after complete consumption of starting material the volatiles were removed under reduced pressure. The residue obtained was taken in dichloromethane (75 ml) and washed with water (2×25 ml). The organic extract was dried over anhydrous sodium sulfate and concentrated to get 6.8 g tetrabutylammonium salt of (2S,5R)-N′-{[5-(hydroxyethyl)-1H-tetrazol-1-yl]acetyl}-7-oxo-6-sulfooxy-1,6-diazabicyclo[3.2.1]octane-2-carbohydrazide as thick off-white liquid in 100% yield.

Step 4: Synthesis of sodium salt of (2S,5R)-6-sulfooxy-N′-{[5-(hydroxyethyl)-1H-tetrazol-1-yl]acetyl}-7-oxo-1,6-diazabicyclo[3.2.1]octane-2-carbohydrazide: To an appropriate column was charged 350 g sodium resin (INDION 225 Na) and washed with water (500 ml) followed by 10% tetrahydrofuran in water (500 ml) for conditioning. The solution of (2S,5R)-N′-{[5-(hydroxyethyl)-1H-tetrazol-1-yl]acetyl}-7-oxo-6-sulfooxy-1,6-diazabicyclo[3.2.1]octane-2-carbohydrazide (6.8 g, mmol) in tetrahydrofuran (20 ml) was loaded on resin column and eluted slowly with 10% tetrahydrofuran in water. The fractions containing the product were collected and concentrated under reduced pressure to remove tetrahydrofuran. The resulted aqueous solution was washed with dichloromethane (2×25 ml) and then concentrated under reduced pressure, yielded 3.1 g of sodium salt of (2S,5R)-N′-{[5-(hydroxyethyl)-1H-tetrazol-1-yl]acetyl}-7-oxo-6-sulfooxy-1,6-diazabicyclo[3.2.1]octane-2-carbohydrazide as off-white solid in 71% yield.

Analysis:

Mass: 433.1(M−1), 435.2 (M+1) for free sulfonic acid; for Molecular weight: 456 and Molecular formula: C₁₂H₁₇N₈O₈S.Na.

¹HNMR (DMSO-d₆D₂O exchange): δ 5.33 (s, 2H), 3.99 (m, 1H), 3.85 (s, 1H), 3.78 (d, 2H), 3.12-2.91 (m, 4H), 2.08-1.59 (m, 4H).

Purity by HPLC: 96.54%

The compounds of Examples 39 to 42 (Table 3) were prepared using the procedure described in Example 38 by using corresponding starting reagents in place of {5-[2-(tert-butyl-dimethyl-silanyloxy)-ethyl]-tetrazol-1-yl}-acetic acid hydrazide showed in Table 3.

TABLE 3

| Example No. | Compound | Starting Reagent | ¹HNMR (400 MHz) | Mass (As free acid) |
|---|---|---|---|---|
| 39 | 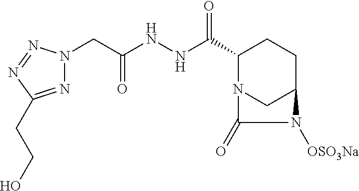 | 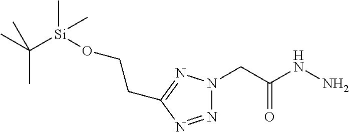 | (DMSO-d₆ D₂O exchange): δ 5.51 (s, 2H), 3.99 (m, 1H), 3.85 (s, 1H), 3.78 (d, 2H), 3.15 (m, 1H), 2.99 (m, 3H), 2.05-1.59 (m, 4H) | 433.1 [M − 1] 435.2 [M + 1] (C₁₂H₁₇N₈O₈SNa) |
| 40 | 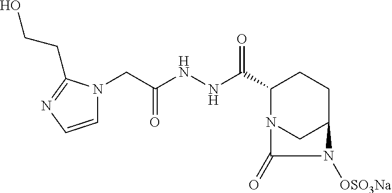 | 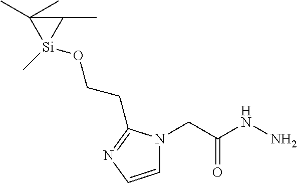 | (DMSO-d₆ D₂O exchange): δ 7.02 (s, 1H), 6.76 (s, 1H), 4.66 (s, 2H), 4.00 (m, 1H), 3.83 (m, 1H), 3.73- 3.67 (m, 2H), 3.14 (m, 1H), 3.02 (m, 1H), 2.78- 2.75 (m, 2H), 2.09-1.63 (m, 4H). | 432.3 [M − 1] 433.3 [M + 1] (C₁₄H₁₉N₆O₈S) |
| 41 | 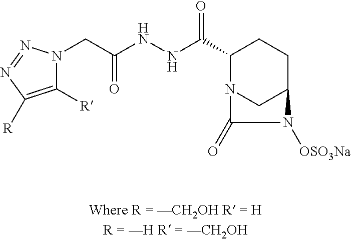 | 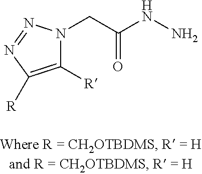 | | 418.3 [M − 1] 420.2 [M + 1] (C₁₂H₁₆N₇O₈S•Na) |
| 42 | 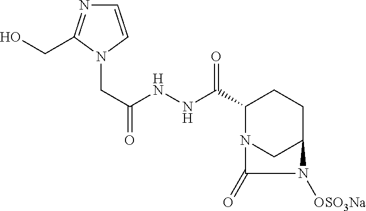 | 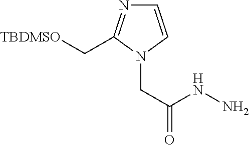 [SMILES: CC(C)(C)[Si](C)(C)OCc1nccn1CC(=O)NN] | (DMSO-d₆): δ 10.01 (brs, 2H), 7.06 (s, 1H), 6.75 (s, 1H), 5.2 (brs, 1H), 4.77 (s, 2H), 4.42 (s, 2H), 3.97 (s, 1H), 3.82 (d, 1H, J = 8 Hz), 3.16 (d, 1H, J = 12 Hz), 2.97 (d, 1H, J = 12 Hz), 2.02 (m, 1H), 1.82-1.57 (m, 3H). | 417.2 [M − 1] 419.3 [M + 1] (C₁₃H₁₇N₆O₈S•Na) |

### Example 43

### Synthesis of sodium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide

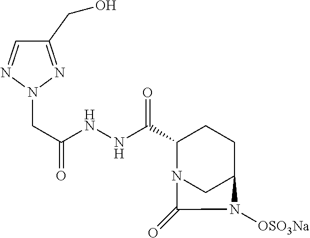

Step 1: Synthesis tetrabutylammonium salt of (2S,5R)-N′-{[4-(tertiary-butyl-dimethyl-silanyloxymethyl)-2H-1,2,3-triazol-2-yl]acetyl}-7-oxo-6-(sulfooxy)-1,6-diazabicyclo[3.2.1]octane-2-carbohydrazide: To a solution of (2S,5R)-7-oxo-6-(sulfooxy)-1,6-diazabicyclo[3.2.1]octane-2-carbohydrazide (3.6 g, 12.8 mmol, prepared as per the reference WO2013030733) in dimethylformamide (18 ml) was added N,N-diisopropylethylamine (6.7 ml) under stirring at 25-30° C. The lithium salt of [4-(tertiary-butyl-dimethyl-silanyloxy methyl)-2H-1,2,3-triazol-2-yl]acetic acid (3.5 g, 12.8 mmol, product from preparation 37) was added as solid under stirring followed by EDC.HCl (3.7 g, 19.3 mmol) and HOBT (1.95 g, 12.7 mmol) at 25-30° C. The progress of reaction was monitored by TLC (chloroform: methanol, 9:1). After complete consumption of starting material a solution of tetrabutyl ammonium acetate (5.8 g, 19.2 mmol) in water (20 ml) was added and stirred for 1 hour. Dimethylformamide was distilled out completely and co-evaporated with xylene (2×25 ml). The concentrated mass thus obtained was poured on to water (36 ml) containing N-methyl morpholine (1 ml) under stirring and extracted with DCM (2×40 ml). The organic extracts were combined and washed with water (1×25 ml), dried over anhydrous sodium sulfate. The volatiles were removed under reduced pressure to get 4.5 g of crude compound which was further purified by column chromatography (100-200 mesh size silica gel) using dichloromethane: methanol as an eluent. Pure fractions were collected and concentrated to yield 1.8 g of tetrabutylammonium salt of (2S,5R)-N′-{[4-(tertiary-butyl-dimethyl-silanyloxymethyl)-2H-1,2,3-triazol-2-yl]acetyl}-7-oxo-6-(sulfooxy)-1,6-diazabicyclo[3.2.1]octane-2-carbohydrazide in 14% yield.

Analysis:

Mass: 534.4 (M−1) as free acid; for Molecular weight: 774 and Molecular formula: C₃₄H₆₆N₈O₈SSi.

Step 2: Synthesis tetrabutylammonium salt of (2S,5R)-N′-([4-(hydroxymethyl)-2H-1,2,3-triazol-2-yl]acetyl}-7-oxo-6-(sulfooxy)-1,6-diazabicyclo[3.2.1]octane-2-carbohydrazide: A solution of tetrabutylammonium fluoride (4.4 mL, 4.3 mmol, 1M solution in tetrahydrofuran) was slowly added to a pre-cooled solution of tetrabutylammonium salt of (2S,5R)-N′-{[4-(tertiary-butyl-dimethyl-silanyloxy methyl)-2H-1,2,3-triazol-2-yl]acetyl}-7-oxo-6-(sulfooxy)-1,6-diazabicyclo[3.2.1]octane-2-carbohydrazide (1.7 g, 2.19 mmol) in tetrahydrofuran (17 ml) at 0° C. under stirring. The reaction mixture was further allowed to stir at ambient temperature. The progress of the reaction was monitored by TLC (chloroform: methanol, 8:2). After completion the reaction mass was concentrated on Rota-evaporator and purified by using column chromatography (100-200 mesh size silica gel) using dichloromethane: methanol (9:1) as an eluent, pure fractions were collected and concentrated to get 0.8 g of tetrabutylammonium salt of (2S,5R)-N′-([4-(hydroxymethyl)-2H-1,2,3-triazol-2-yl]acetyl}-7-oxo-6-(sulfooxy)-1,6-diazabicyclo[3.2.1]octane-2-carbohydrazide in 56% yield.

Analysis:

Mass: 418.3 (M−1) as free acid; for Molecular weight: 660 and Molecular formula: C₂₈H₅₂N₈O₈S.

Step 3: Synthesis of sodium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide: Appropriate column was loaded with Indion 225 sodium resin (200 g) and eluted millipore water (250 ml) followed by 10% tetrahydrofuran in water (250 ml). A solution of tetrabutylammonium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide (0.7 g, 1.0 mmol) dissolved in tetrahydrofuran (1 ml) and water (10 ml) mixture was loaded on column and eluted with 10% tetrahydrofuran in water. The pure fractions were collected and concentrated on rota evaporator under high vacuum at about 35° C. to obtain 0.370 g of sodium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide, 0.370 g, 79% yield.

Analysis:

Mass: 418.3(M−1), 420.3 (M+1) as free acid; for Molecular weight: 441 and Molecular formula: C₁₂H₁₆N₇O₈S.Na.

¹HNMR (DMSO-d₆, D₂O): δ 10.16 (br s, 2H), 7.68 (s, 1H), 5.27 (m, 1H), 5.17 (s, 2H), 4.52 (d, 2H, J=5.2 Hz), 3.99 (s, 1H), 3.83 (d, 1H, J=7.2 Hz), 3.17 (d, 1H, J=12 Hz), 2.98 (d, 1H, J=11.2 Hz), 2.05-2.0 (m, 2H), 1.84-1.59 (m, 2H).

### Example 44

### Synthesis of (2S,5R)-N-{2-[5-(aminomethyl)-2H-tetrazol-2-yl]ethoxy}-7-oxo-6-(sulfooxy)-1,6-diazabicyclo[3.2.1]octane-2-carboxamide

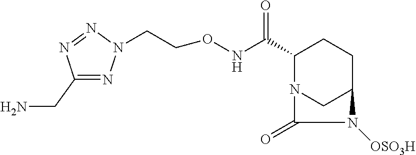

Step 1: Synthesis of tert-butyl [(2-{2-[(1,3-dioxo-1,3-dihydro-2H-isoindol-2-yl)oxy]ethyl}-2H-tetrazol-5-yl)methyl]carbamate: To a solution of 2-(2-bromoethoxy)isoindoline-1,3-dione (20 g, 74 mmol, prepared according to the procedure described in step 1 of preparation 38), (1H-tetrazol-5-ylmethyl)-carbamic acid tert-butyl ester (7.66 g, 74 mmol, prepared as per procedure described in step 2 of preparation 19) in dimethylformamide (100 ml) was added cesium carbonate (24.13 g, 74 mmol) lot wise under stirring at 25° C. After 16 hours, the reaction mixture was filtered and filtrate was slowly poured into chilled water (700 ml) and stirred for 30 minutes. The precipitated compound was filtered and washed with water (100 ml). The solid compound was dried at 35° C. for 2 hours under high vacuum, to provide 16 g of tert-butyl [(2-{2-[(1,3-dioxo-1,3-dihydro-2H-isoindol-2-yl)oxy]ethyl}-2H-tetrazol-5-yl)methyl]carbamate in 55% yield.

Analysis:

Mass: 389.4 (M+1); for Molecular weight: 388 and Molecular formula: C₁₇H₂₀N₆O₅.

Step 2: Synthesis of [2-(2-Aminooxy-ethyl)-2H-tetrazol-5-ylmethyl]-carbamic acid tert-butyl ester: To a clean dry flask containing tert-butyl [(2-{2-[(1,3-dioxo-1,3-dihydro-2H-isoindol-2-yl)oxy]ethyl}-2H-tetrazol-5-yl)methyl]carbamate (6.5 g, 16 mmol, product from Step 1) in dichloromethane (65 ml)) was added hydrazine hydrate monohydrate (1.25 ml, 25 mmol) under stirring at room temperature. After 2 hours, the progress of the reaction was monitored by thin layer chromatography using mixture of chloroform and methanol as solvent system. After completion of the reaction, the unwanted solid was filtered out and washed with dichloromethane (13 ml). The filtrate was concentrated at 40° C. under high vacuum to obtain 4.3g of [2-(2-aminooxy-ethyl)-2H-tetrazol-5-ylmethyl]-carbamic acid tert-butyl ester, which was used as such without any further purification.

Step 3: Synthesis of (2S,5R)-N-{[2-(5-{[(tert-butoxycarbonyl)amino]methyl}-2H-tetrazol-2-yl)ethoxy]}-6-benzyloxy-7-oxo-1,6-diazabicyclo[3.2.1]octane-2-carboxamide: To a solution of sodium salt of (2S,5R)-6-(benzyloxy)-7-oxo-1,6-diaza-bicyclo[3.2.1]octane-2-carboxylic acid (4.0 g, 13.4 mmol) in dimethylformamide (20 ml) at ambient temperature was added EDC.HCl (4.16 g, 21.7 mmol), followed by N-methyl morpholine (4.62 ml, 40 mmol) and HOBT (2 g, 13.4 mmol) under continuous stirring. After 5 minutes he reaction mixture was cooled to 15° C. and a solution of [2(2-aminooxy-ethyl)-2H-tetrazol-5-ylmethyl]-carbamic acid tert-butyl ester (4.3 g, 13.4 mmol product of Step 2) in dimethylformamide (4.3 ml) was slowly added under stirring and allowed to attain room temperature. After 16 hours, the completion of reaction was confirmed by performing the thin layer chromatography using mixture of chloroform and methanol (9:1) as solvent system. The resulted reaction mixture was slowly poured into chilled water (160 ml) and mixture was extracted with ethyl acetate (2×40 ml). The ethyl acetate layer was washed with water (1×40 ml) and brine (1×40 ml). The collective organic layer was dried over anhydrous sodium sulfate and evaporated under reduced pressure to dryness, which was purified by column chromatography over silica gel using 30% acetone in hexane as an eluent. The compound containing fractions were collected and on concentration of the combined fractions 4 g of (2S,5R)-N-{[2-(5-{[(tert-butoxycarbonyl)amino]methyl}-2H-tetrazol-2-yl)ethoxy]}-6-benzyloxy-7-oxo-1,6-diazabicyclo[3.2.1]octane-2-carboxamide was obtained in 53% yield.

Analysis:

Mass: 517.5 (M+1); for Molecular weight: 516 and Molecular formula: C₂₃H₃₂N₈O₆.

Step 4: Synthesis of tetrabutyl ammonioum salt of (2S,5R)-N-{[2-(5-{[(tert-butoxycarbonyl) amino]methyl-2H-tetrazol-2-yl)ethoxyl}-6-sulfooxy-7-oxo-1,6-diazabicyclo[3.2.1]octane-2-carboxamide: To a solution of (2S,5R)-N-{[2-(5-{[(tert-butoxycarbonyl)amino]methyl}-2H-tetrazol-2-yl)ethoxy]}-6-benzyloxy-7-oxo-1,6-diazabicyclo[3.2.1]octane-2-carboxamide (4 g, 7.7 mmol, product from Step 3) in dimethylformamide (20 ml) and dichloromethane (20 ml) was added palladium over carbon (10%, 1.0 g) under nitrogen atmosphere. The reaction mixture was flushed with hydrogen gas and stirred for 3 hours under hydrogen pressure (50 psi). The progress of reaction was monitored by performing thin layer chromatography using mixture of chloroform: methanol (9:1) as solvent system. After completion of the reaction, the resulted reaction mixture was filtered through celite bed and bed was washed with a mixture of dichloromethane in dimethylformamide (20 ml, 1:1). The filtrate was concentrated, and the resulted residue was dissolved in pyridine (28 ml) and to the clear solution was added pyridine sulfur trioxide complex (6.16 g, 38.7 mmol). The suspension was stirred at a temperature of 25° C. for overnight. The suspension was filtered and the solids were washed with dichloromethane (2×25 ml). The filtrate was evaporated under vacuum and the residue was stirred in 0.5 N aqueous potassium dihydrogen phosphate solution (200 ml) for 1 hour. The resulted solution was washed with ethyl acetate (2×100 ml) and layers were separated. Tetrabutylammonium hydrogen sulphate (2.62 g, 7.7 mmol) was added to the aqueous layer and stirred for 1 hour at about 25° C. The mixture was extracted with dichloromethane (2×100 ml). The combined organic extract was washed with water (50 ml) and dried over anhydrous sodium sulfate. The solvent was evaporated under reduced pressure and the residue was purified by column chromatography over silica gel using 5% methanol in chloroform as an eluent. The combined fractions containing compound were concentrated to obtain 3.8 g of tetrabutyl ammonium salt of (2S,5R)-N-{[2-(5-{[(tert-butoxycarbonyl)amino]methyl}-2H-tetrazol-2-yl)ethoxy]}-6-sulfooxy-7-oxo-1,6-diazabicyclo[3.2.1]octane-2-carboxamide in 65% yield.

Analysis:

Mass: 505.2 (M−1) as free acid; for Molecular weight: 747 and Molecular formula: C₃₂H₆₁N₉O₉S.

Step 5: Synthesis of (2S,5R)-N-{2-[5-(aminomethyl)-2H-tetrazol-2-yl]ethoxyl-7-oxo-6-(sulfooxy)-1,6-diazabicyclo[3.2.1]octane-2-carboxamide: To a solution of tetrabutyl ammonium salt of (2S,5R)-N-{[2-(5-{[(tert-butoxycarbonyl)amino]methyl}-2H-tetrazol-2-yl)ethoxy]}-6-sulfooxy-7-oxo-1,6-diazabicyclo[3.2.1]octane-2-carboxamide, (0.300 g, 0.401 mmol, product form Step 4) in dichloromethane (1.5 ml) was slowly added trifluoroacetic acid (1.5 ml) by syringe at 0° C. over a period of 5 minutes under stirring. After 2 hours, the progress of reaction was monitored by thin layer chromatography using mixture of chloroform and methanol (9:1) as solvent system. After complete conversion the solvents were removed below 40° C. under high vacuum to provide a residue, which was triturated with diethyl ether (2×5 ml) and with dichloromethane (2×5 ml). The obtained solid was dried under vacuum to provide 0.130 g (2S,5R)-N-{2-[5-(aminomethyl)-2H-tetrazol-2-yl]ethoxy}-7-oxo-6-(sulfooxy)-1,6-diazabicyclo[3.2.1]octane-2-carboxamide in 80% yield.

Analysis:

Mass: 405.2 (M−1) as for free acid; for Molecular weight: 406.37 and Molecular formula: C₁₁H₁₈N₈O₇S;

¹H -NMR (400 MHz, DMSO-d₆): δ 11.53 (s, 1H), 8.53 (s, 3H), 4.99 (t, 2H, J=3.2 Hz), 4.42 (s, 2H), 4.35 (t, 2H, J=5.2 Hz), 4.00 (s, 1H), 3.70 (d, 1H, J=4 Hz), 2.99 (s, 2H), 1.66-1.96 (m, 4H);

Purity as determined by HPLC: 93.95%.

### Example 45

### Synthesis sodium salt of (2S,5R)-7-oxo-6-(sulfooxy)-N-[2-(2H-1,2,3-triazol-2-yl)ethoxyl-1,6-diazabicyclo[3.2.1]octane-2-carboxamide

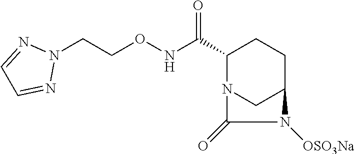

Step 1: Synthesis mixture of 2-[2-(2H-1,2,3-triazol-2-yl)ethoxy]-1H-isoindole-1,3(2H)-dione and 2-[2-(1H-1,2,3-triazol-1-yl)ethoxy]-1H-isoindole-1,3(2H)-dione: To a clean dry flask containing suspension of 2-(2-bromoethoxy)isoindoline-1,3-dione (30 g, 0.111 mol), 1H-1,2,3 triazole (7.66 g, 111 mmol) in dimethylformamide (150 ml) was added cesium carbonate ((36 g, 111 mmol) portion wise at room temperature under stirring. After 17 hours, the reaction mixture was filtered and filtrate was slowly poured into chilled water (1050 ml) under stirring. After 30 minutes of stirring, the separated precipitates were collected by filtration and washed with water (100 ml). The collected precipitates were dried at 40° C. for 2 hours under high vacuum to obtain 18.5 g of a mixture of 2-[2-(2H-1,2,3-triazol-2-yl)ethoxy]-1H-isoindole-1,3(2H)-dione and 2-[2-(1H-1,2,3-triazol-1-yl)ethoxy]-1H-isoindole-1,3(2H)-dione as a white compound in 65% yield.

Analysis:

Mass: 259.2 (M+1); for Molecular weight: 258 and Molecular formula: C₁₂H₁₀N₄O₃.

Step 2: Synthesis of mixture of 2-[2-(aminooxy)ethyl]-2H-1,2,3-triazole and 1-[2-(aminooxy)ethyl]-1H-1,2,3-triazole: To a solution of mixture of 2-(2-(2H-1,2,3-triazol-2-yl)ethoxy)isoindoline-1,3-dione and 2-(2-(1H-1,2,3-triazol-1-yl)ethoxy)isoindoline-1,3-dione (13 g, 50.3 mmol, obtained from Step 1) in dichloromethane (130 ml) was added hydrazine hydrate (3.7 ml, 75.7 mmol) under stirring at room temperature. After 2 hours, the progress of reaction was monitored by thin layer chromatography using mixture of chloroform and methanol as solvent system. After completion of the reaction the unwanted solid was filtered out and washed with dichloromethane (25 ml). The collective filtrates were concentrated at 40° C. under high vacuum to dryness to provide 6.5 g of a mixture of 2-[2-(aminooxy)ethyl]-2H-1,2,3-triazole and 1-[2-(aminooxy)ethyl]-1H-1,2,3-triazole, which was used as such in the next step without any further purification.

Step 3: Synthesis of (2S,5R)-6-(benzyloxy)-7-oxo-N-[2-(2H-1,2,3-triazol-2-yl)ethoxy]-1,6-diazabicyclo[3.2.1]octane-2-carboxamide and (2S,5R)-6-(benzyloxy)-7-oxo-N-[2-(1H-1,2,3-triazol-1-yl)ethoxy]-1,6-diazabicyclo[3.2.1]octane-2-carboxamide: To a solution of sodium salt of (2S,5R)-6-(benzyloxy)-7-oxo-1,6-diaza-bicyclo[3.2.1]octane-2-carboxylic acid (15 g, 54.3 mmol, prepared as per the procedure disclosed in International Patent Application No. PCT/IB2013/059264) in dimethylformamide (75 ml) was added EDC.HCl (15.6 g, 81.6 mmol) at ambient temperature under stirring. Then N-methyl morpholine (16.5 ml, 0.150 moles) was added followed by HOBT (7.73 g, 50.7 mmol) and reaction mixture was stirred for 5 minutes at ambient temperature. To this reaction mixture was added a solution of mixture of 2-[2-(aminooxy)ethyl]-2H-1,2,3-triazole and 1-[2-(aminooxy)ethyl]-1H-1,2,3-triazole (6.5 g, product from Step 2) in dimethylformamide (6.5 ml) under stirring at 15° C. and then allowed to attain room temperature. The progress of reaction was monitored by thin layer chromatography using mixture of chloroform and methanol (9:1) as solvent system. After completion of the reaction (17 hours), the resulted reaction mixture was slowly poured into chilled water (500 ml) and extracted with ethyl acetate (2×150 ml). The combined organic layer was washed with brine (75 ml) and organic layer was dried over anhydrous sodium sulfate, concentrated on rotavapour to provide 17.5 g of crude compound. It was purified by column chromatography (silica gel 60-120 mesh) using mixture of hexane and acetone as an eluent. The upper spot containing fractions were collected at 30-40% concentration of acetone in hexane and concentrated to dryness under vacuum to provide7.5 g of (2S,5R)-6-(benzyloxy)-7-oxo-N-[2-(2H-1,2,3-triazol-2-yl)ethoxy]-1,6-diazabicyclo[3.2.1]octane-2-carboxamide. Similarly lower spot containing fractions collected at 40-50% concentration of acetone in hexane were concentrated to dryness under vacuum to provide 5.2 g of of (2S,5R)-6-(benzyloxy)-7-oxo-N-[2(1H-1,2,3-triazol-1-yl)ethoxyl -1,6-diazabicyclo[3.2.1]octane-2-carboxamide. In total, 12.7 g was obtained in 70% yield.

Analysis:

Mass: 385.3 (M−1); for Molecular weight: 386 and Molecular formula: C₁₈H₂₂N₆O₄.

Step 4: Synthesis Tetrabutyl ammonium salt of (2S,5R)-7-oxo-6-(sulfooxy)-N-[2-(2H-1,2,3-triazol-2-yl)ethoxy]-1,6-diazabicyclo[3.2.1]octane-2-carboxamide: To a solution of (2S,5R)-6-(benzyloxy)-7-oxo-N-[2-(2H-1,2,3-triazol-2-yl)ethoxy]-1,6-diazabicyclo[3.2.1]octane-2-carboxamide (4 g, 10.3 mmol, upper spot as per thin layer chromatography in Step 3) in dimethylformamide (20 ml) and dichloromethane (20 ml) was added palladium over carbon (10%, 1.0 g) under nitrogen atmosphere. The reaction mixture was flushed with hydrogen gas and stirred for 3 hour under hydrogen pressure (55 psi). The progress of reaction was monitored by thin layer chromatography using mixture of chloroform and methanol (9:1) as solvent system. After complete conversion, the reaction mixture was filtered through celite bed and washed with a mixture of dichloromethane and dimethylformamide (20 ml, 1:1). The collected filtrate was evaporated under reduced pressure to dryness. The intermediate thus obtained was dissolved into dimethylformamide (20 ml) and dimethylformamide sulfur trioxide complex (2.4 g, 15.6 mmol) was added under stirring at 0° C. The reaction mixture was allowed to attain ambient temperature and stirred further for 1 hour. The completion of reaction was monitored by performing thin layer chromatography using mixture of chloroform and methanol as solvent system. After complete conversion, the reaction mixture was cooled to 0° C. and then a solution of tetra butyl ammonium acetate (5 g, 16.5 mmol) in water (17 ml) was slowly added under stirring. After 1 hour, the reaction mixture was concentrated to dryness in vacuum and co-evaporated with xylene (2×30 ml) to dimethylformamide free mass. To this concentrated mass, water (40 ml) was added and then extraction with dichloromethane was carried (2×40 ml). The collective organic layer was dried on anhydrous sodium sulfate and concentrated to dryness to provide 8.5 g of crude compound. It was purified using column chromatography (silcagel 60-120) by using mixture of dichloromethane and methanol as an eluent. The pure compound was isolated at 5% concentration of methanol in dichloromethane; the collective fractions were collected and evaporated to obtain 3.5 g of tetrabutyl ammonium salt of (2S,5R)-7-oxo-6-(sulfooxy)-N-[2-(2H-1,2,3-triazol-2-yl)ethoxy]-1,6-diazabicyclo[3.2.1]octane-2-carboxamide in 55% yield.

Analysis:

Mass: 375.2 (M−1, for free acid) for Molecular weight: 617 and Molecular formula: C₂₇H₅₁N₇O₇S.

Step 5: Synthesis sodium salt of (2S,5R)-7-oxo-6-(sulfooxy)-N-[2-(2H-1,2,3-triazol-2-yl)ethoxy]-1,6-diazabicyclo[3.2.1]octane-2-carboxamide: A column loaded with Indion 225 Na resin was eluted with water (500 ml) and 10% tetrahydrofuran in water (250 ml). A solution of tetrabutyl ammonium salt of (2S,5R)-7-oxo-6-(sulfooxy)-N-[2-(2H-1,2,3-triazol-2-yl)ethoxy]-1,6-diazabicyclo[3.2.1]octane-2-carboxamide (3.5 g, 5.6 mmol, product from Step 4) in tetrahydrofuran (7 ml) diluted up to 70 ml using water was loaded on Indion 225 Na ion exchange resin column and then was eluted using 10% tetrahydrofuran in water. The fractions containing compound were confirmed by performing thin layer chromatography using mixture of chloroform and methanol as solvent system. The collective fractions containing compound was concentrated under vacuum till dryness to provide 1.8 g of sodium salt of (2S,5R)-7-oxo-6-(sulfooxy)-N-[2-(2H-1,2,3-triazol-2-yl)ethoxy]-1,6-diazabicyclo[3.2.1]octane-2-carboxamide as a white solid in 80% yield.

Analysis:

Mass: 375.2 (M−1) for free acid, for Molecular weight: 398.3 and Molecular formula: C₁₁H₁₅N₆O₇S.Na;

¹H NMR (400 MHz, DMSO-d₆): δ 11.5 (1H, s), 7.77 (2H, s), 4.64 (2H, t, J=5.6, 4.8 Hz), 4.23 (2H, t, J=5.2 Hz), 3.96 (1H, s), 3.66 (1H, d, J=6 Hz), 3.05 (1H, J=12 Hz), 2.94 (1H, d, J=11.6 Hz), 1.93-1.91 (1H, m), 1.82 (1H, m), 1.67-1.64 (2H, m); Purity as determined by HPLC: 94.75%.

The compound of Example 46 (Table 4) was prepared using the procedure described in Example 44 and corresponding starting reagents in place of (2S,5R)-N-{[2-(5-{[(tert-butoxycarbonyl)amino]methyl}-2H-tetrazol-2-yl)ethoxy]}-6-benzyloxy-7-oxo-1,6-diazabicyclo[3.2.1]octane-2-carboxamide. The compounds of Examples 47 to 57 (Table 4) were prepared using the procedure described in Example 45 by using starting materials described in Table 4 in place of 2-[2-(aminooxy)ethyl]-2H-1,2,3-triazole and 1-[2-(aminooxy)ethyl]-1H-1,2,3-triazole. The isomeric mixtures were separated at benzyl stage product (step 3) to single isomers using silicagel column chromatography or else further followed as per the procedures described in Example 45. For Examples 55 to 57, followed the deprotection of tert-butyldimethylsilyloxy (TBDMS) group using tetrabutylammonium fluoride (TBAF) in tetrahydrofuran and then converted them to corresponding sodium salt using the procedure described for Example 45.

TABLE 4

| Ex- | | | | |
|---|---|---|---|---|
| am- | | | | |
| ple | | | | Mass |
| No. | Compound | Starting Reagent | ¹HNMR in DMSO-d₆ (400 MHz) | (As free acid) |
| 46 | 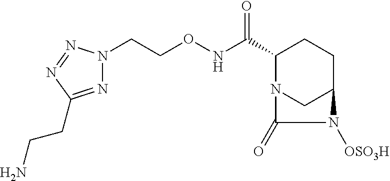 | 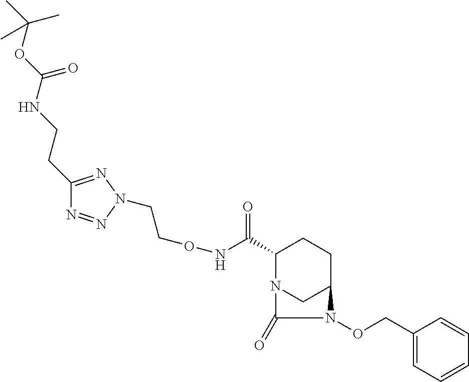 [SMILES: CC(C)(C)OC(=O)NCCc1nnn(CCONC(=O)[C@@H]2CC[C@@H]3CN2C(=O)N3OCc2ccccc2)n1] | δ 11.25 (bs, 1H), 7.90 (bs, 3H), 4.90 (s, 2H), 4.33 (t, 1H, J = 4.8 Hz), 3.99 (s, 1H), 3.69-3.68 (d, 1H, 6 Hz), 3.32-3.18 (m, 5H), 2.98 (s, 2H), 1.95-1.85 (m, 2H), 1.72-1.62 (m, 2H) | 419.2 [M − 1] (C₁₂H₂₀N₈O₇S) |
| 47 | 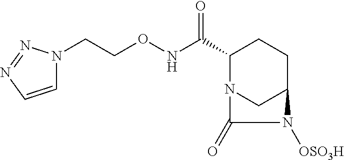 | 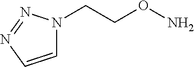 | δ 11.5 (1H, br s), 8.29 (1H, s), 7.71 (1H, s), 4.60 (2H, t, J = 4.4 Hz), 4.13 (2H, t, J = 4.8 Hz), 3.97 (1H, s), 3.71 (1H, d, J = 6 Hz), 3.05 (1H, d, J = 12 Hz), 2.96 (1H, d, J = 11.6 Hz), 1.98-1.83 (2H, m), 1.73- 1.63 (2H, m) | 375.2 [M − 1] (C₁₁H₁₅N₆O₇SNa) |
| 48 | 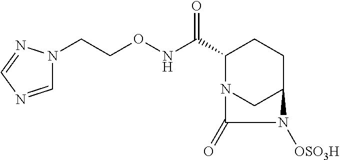 | 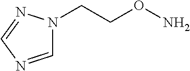 | δ 11.6 (1H, br s), 8.62 (1H, s), 7.95 (1H, s), 4.39 (2H, t, J = 4.4, 4.8 Hz), 4.10 (2H, t, J = 5.2, 4.4 Hz), 3.97 (1H, s), 3.71 (1H, d, J = 6 Hz), 2.99 (2H, dd, J= 10.8, 11.2 Hz), 1.98-1.83 (2H, m), 1.73-1.63 (2H, m) | 375.2 [M − 1] (C₁₁H₁₅N₆O₇SNa) |
| 49 | 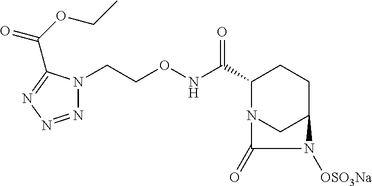 | 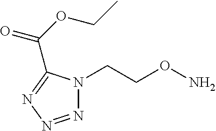 | δ 11.50 (s, 1H), 4.90-5.03 (m, 2H), 4.29- 4.45 (m, 3H), 3.96 (s, 1H), 3.56-3.66 (m, 2H), 2.96-2.99 (m, 2H), 1.62-1.93 (m, 4H), 1.31-1.36 (m, 3H). | 448.3 (C₁₃H₁₈N₇O₉SNa) |
| 50 | 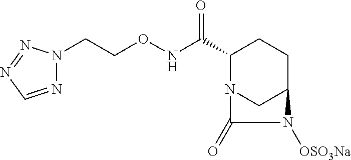 | 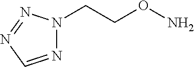 | δ 11.50 (s, 1H), 8.97 (s, 1H), 4.92-4.94 (m, 2H), 4.31 (t, 2H, J = 5.2 Hz), 3.96 (s, 1H), 3.58-3.66 (m, 1H), 3.12-3.16 (m, 1H), 2.95-2.98 (m, 2H), 1.64-1.90 (m, 4H). | 376.1 [M − 1] (C₁₀H₁₄N₇O₇SNa) |
| 51 | 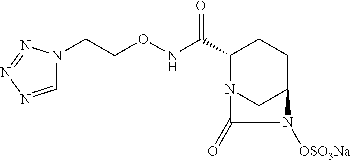 | 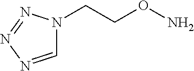 | δ 11.60 (s, 1H), 9.53 (s, 1H), 4.71 (t, 2H, J = 8.4 Hz), 4.20 (s, 2H, J = 4.0 Hz), 3.99 (s, 1H), 3.75 (d, 1H, J = 6.4 Hz), 3.58-3.65 (m, 1H), 3.00 (s, 2H), 1.67-2.02 (m, 3H). | 376.1 [M − 1] (C₁₀H₁₄N₇O₇SNa) |
| 52 | 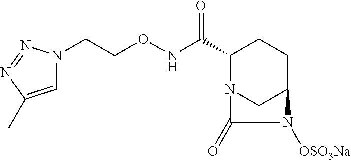 | 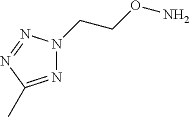 | δ 11.50 (s, 1H), 4.84-4.86 (m, 2H), 4.30 (t, 2H, J = 3.2 Hz), 3.98 (s, 1H), 3.58-3.69 (m, 2H), 2.95-3.04 (m, 2H), 2.49 (s, 3H), 1.66-1.93 (m, 3H). | 390.3 [M − 1] (C₁₁H₁₆N₇O₇SNa) |
| 53 | 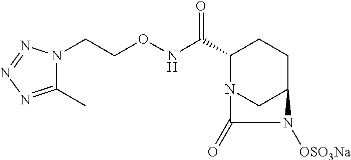 | 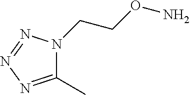 | δ 11.50 (s, 1H), 4.57-4.59 (m, 2H), 4.19- 4.21 (m, 2H), 3.98 (s, 1H), 3.60-3.69 (m, 1H), 2.98 (s, 1H), 2.53 (s, 3H), 1.65-1.96 (m, 4H). | 390.3 [M − 1] (C₁₁H₁₆N₇O₇SNa) |
| 54 | 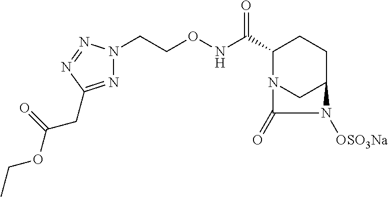 | 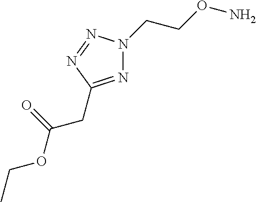 | δ 4.90 (t, 2H, J = 3.2 Hz), 4.29 (t, 2H, J = 4.8 Hz), 4.13-4.07 (q, 2H), 4.03 (s, 2H), 3.96 (s, 1H), 3.66 (d, 1H, J = 6.0 Hz), 3.59-3.56 (m, 1H), 3.00-2.93 (m, 2H), 1.93-1.65 (m, 4H), 1.19-1.15 (t, 3H, J = 14.8 Hz). | 462.3 [M − 1] (C₁₄H₂₀N₇O₉SNa |
| 55 | 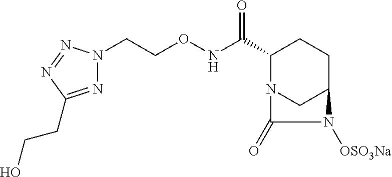 | 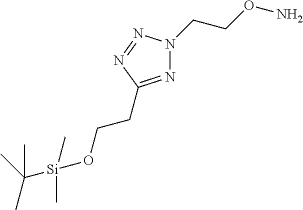 [SMILES: CC(C)(C)[Si](C)(C)OCCc1nnn(CCON)n1] | δ 11.41 (bs, 1H), 4.86-4.84 (m, 2H), 4.80 (m, 1H), 4.22 (s, 2H), 3.95 (s, 1H), 3.79-3.75 (m, 2H), 3.62-3.59 (m, 2H), 3.58-3.32 (m, 1H), 2.99-2.95 (m, 2H), 2.89-2.87 (m, 1H), 1.90-1.62 (m, 4H). | 420.2 [M − 1] C₁₂H₁₈N₇O₈SNa) |
| 56 | 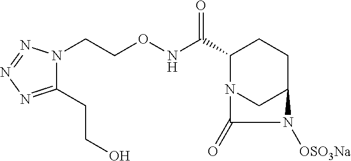 | 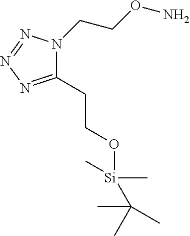 [SMILES: CC(C)(C)[Si](C)(C)OCCc1nnnn1CCON] | δ 11.51 (bs, 1H), 4.60 (t, 2H, J = 4.8 Hz), 4.15 (t, 1H, J = 4.4 Hz), 3.95 (s, 1H), 3.95 (s, 1H), 3.74 (t, 2H, J = 6.4 Hz), 3.65-3.56 (m, 2H), 3.04 (t, 2H, J = 6.4 Hz), 2.94- 2.91 (m, 2H), 1.93-1.61 (m, 4H). | 420.2 [M − 1] (C₁₂H₁₈N₇O₈SNa) |
| 57 | 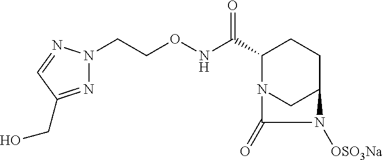 | 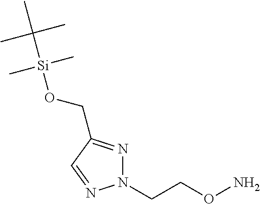 | δ 11.5 (br s, 1H), 7.65 (s, 1H), 5.25 (br s, 1H), 4.60 (t, 2H, J = 4.8 Hz, J = 10 Hz), 4.51 (s, 2H), 4.16 (s, 2H), 3.96 (s, 1H), 3.59 (s, 1H), 3.25 (m, 1H), 2.90 (d, 1H, J = 11.2 Hz), 1.92-1.90 (m, 2H), 1.81- 1.64 (m, 2H). | 405.3 [M − 1] 407.3 [M + 1] (C₁₂H₁₇N₆O₈SNa) |

### Biological Activity Data

The biological activity of representative compounds according to the invention against various bacterial strains was investigated. The Minimum Inhibitory Concentration (MIC) determination for the combinations was carried out in Muller Hinton Agar (MHA) (BD, USA) according to Clinical and Laboratory Standards Institute (CLSI) recommendations, (Clinical and Laboratory Standards Institute (CLSI), Performance Standards for Antimicrobial Susceptibility Testing, 20^{th} Informational Supplement, M 100-S20, Volume 30, No. 1, 2010). In short, the test strains were adjusted to deliver about 10⁴ CFU per spot with a multipoint inoculator (Applied Quality Services, UK). The plates were poured with MHA containing doubling concentration range of representative compounds according to present invention. The plates were inoculated and were incubated at 35° C. for 18 hours. MICs were read as the lowest concentration of drug that completely inhibited bacterial growth. The Table 5 depicts the antibacterial activity profile of compounds according to present invention against various multidrug resistant bacterial strains. These compounds when tested alone exhibited lower MIC values in comparison to reference agent.

The combinations of compounds according to present invention were also tested for their antibacterial activity in combination with Ceftazidime. The plates were poured with MHA containing doubling concentration range of Ceftazidime in combination with constant concentration (4 mcg/ml) of representative compounds of Formula (I). The Table 6 shows the MIC values of Ceftazidime in presence of compounds according to the invention (at 4 mcg/ml). As shown in Table 6, the MIC value of Ceftazidime was significantly lowered in presence of compounds according to the invention.

TABLE 5
Antibacterial activity of representative compounds
according to invention (expressed as MICs (mcg/ml).

| | Strains | | | | | | |
|---|---|---|---|---|---|---|---|
| | | E. coli | E. coli | | | | |
| | K. pneumoniae | NCTC | NCTC | E. coli | E. coli | K. pneumoniae | K. pneumoniae |
| Compounds | ATCC 700603 | 13352 | 13353 | M 50 | 7 MP | H521 | H525 |
| Example 1 | >32 | >32 | >32 | >32 | >32 | >32 | >32 |
| Example 2 | >32 | 0.5 | 0.25 | 0.5 | 1 | 1 | 1 |
| Example 3 | >32 | 0.5 | 0.5 | 0.5 | 2 | 1 | 1 |
| Example 4 | >32 | 0.5 | 0.5 | 0.5 | 1 | 1 | 1 |
| Example 5 | >32 | 0.5 | 0.5 | 1 | 2 | 2 | 2 |
| Example 6 | >32 | 1 | 1 | 1 | 2 | 2 | 2 |
| Example 7 | >32 | 0.5 | 0.5 | 0.5 | 1 | 1 | 1 |
| Example 8 | >32 | 0.5 | 0.5 | 0.5 | 2 | 1 | 1 |
| Example 9 | >32 | 0.5 | 0.25 | 0.25 | 1 | 4 | 1 |
| Example 10 | >32 | 0.5 | 0.5 | 0.5 | 1 | 2 | 4 |
| Example 11 | >32 | 1 | 1 | 1 | 4 | 32 | >32 |
| Example 12 | >32 | 0.5 | 1 | 2 | 2 | 4 | 4 |
| Example 13 | >32 | 1 | 1 | 2 | 2 | 32 | 32 |
| Example 14 | >32 | 1 | 1 | 0.5 | 1 | 4 | 4 |
| Example 15 | >32 | 0.5 | 0.5 | 0.5 | 1 | 2 | 2 |
| Example 16 | >32 | 1 | 1 | 1 | 2 | 2 | 2 |
| Example 17 | >32 | 0.25 | 0.25 | 0.25 | 0.5 | 1 | 1 |
| Example 18 | >32 | 0.5 | 0.25 | 0.5 | 1 | 1 | 1 |
| Example 20 | >32 | 0.5 | 0.5 | 0.5 | 1 | 2 | 2 |
| Example 21 | >32 | 2 | 2 | 2 | 4 | 8 | 8 |
| Example 24 | >32 | 1 | 0.5 | 0.5 | 2 | 2 | 2 |
| Example 25 | >32 | 1 | 1 | 0.5 | 2 | 1 | 1 |
| Example 26 | >32 | 0.5 | 0.5 | 0.5 | 1 | 1 | 1 |
| Example 27 | >32 | 1 | 1 | 1 | 2 | 2 | 2 |
| Example 28 | >32 | 0.5 | 0.25 | 0.25 | 1 | 0.5 | 0.5 |
| Example 30 | >32 | 0.5 | 0.25 | 0.5 | 1 | 0.5 | 0.5 |
| Example 31 | >32 | 0.5 | 0.5 | 0.5 | 1 | 1 | 1 |
| Example 32 | >32 | 0.5 | 0.25 | 0.5 | 1 | 0.5 | 0.5 |
| Example 33 | >32 | 1 | 0.5 | 0.5 | 1 | 4 | 0.5 |
| Example 34 | >32 | 1 | 1 | 1 | 4 | 2 | 1 |
| Example 35 | >32 | 1 | 1 | 1 | 4 | 2 | 2 |
| Example 36 | >32 | 2 | 2 | 2 | 4 | 4 | 4 |
| Example 37 | >32 | 2 | 1 | 1 | 4 | 4 | 4 |
| Example 38 | >32 | 0.25 | 0.25 | 0.25 | 1 | 0.5 | 0.5 |
| Example 39 | >32 | 0.5 | 0.5 | 0.5 | 1 | 1 | 2 |
| Example 40 | >32 | 2 | 1 | 2 | 4 | 4 | 2 |
| Example 42 | >32 | 1 | 0.5 | 0.5 | 2 | 2 | 1 |
| Example 43 | >32 | 0.5 | 0.25 | 1 | 2 | 2 | 2 |
| Example 44 | >32 | >32 | >32 | >32 | >32 | >32 | >32 |
| Example 45 | >32 | >32 | >32 | >32 | >32 | >32 | >32 |
| Example 46 | >32 | 8 | 4 | 4 | 32 | 4 | 4 |
| Example 47 | >32 | 8 | 8 | 8 | 32 | >32 | >32 |
| Example 48 | >32 | 16 | 8 | 8 | >32 | >32 | 32 |
| Example 49 | >32 | >32 | >32 | >32 | >32 | >32 | >32 |
| Example 50 | >32 | 8 | 8 | 8 | 32 | >32 | >32 |
| Example 51 | >32 | 8 | 4 | 4 | 16 | >32 | >32 |
| Example 53 | >32 | 8 | 4 | >32 | 32 | 16 | 16 |
| Example 55 | >32 | 32 | 16 | 16 | >32 | >32 | >32 |
| Example 56 | >32 | 8 | 4 | 4 | >32 | 8 | 16 |
| Cefepime | 0.5 | 4 | 32 | >32 | 16 | 32 | 32 |

TABLE 6
Antibacterial activity of representative compounds according to the invention
in combination with Ceftazidime (expressed as MICs (mcg/ml).

| | Strains | | | | | | |
|---|---|---|---|---|---|---|---|
| | | E. coli | E. coli | | | | |
| | K. pneumoniae | NCTC | NCTC | E. coli | E. coli | K. pneumoniae | K. pneumoniae |
| Compounds | ATCC 700603 | 13352 | 13353 | M 50 | 7 MP | H521 | H525 |
| Ceftazidime alone | 16 | 16 | >32 | >32 | >32 | >32 | >32 |
| Ceftazidime + Example | 1 | 4 | 0.25 | 1 | 4 | 16 | 16 |
| 45 (4 mcg/ml) | | | | | | | |
| Ceftazidime + Example | 1 | 2 | 0.25 | 0.25 | 4 | 16 | 8 |
| 48 (4 mcg/ml) | | | | | | | |
| Ceftazidime + Example | 2 | 8 | 0.5 | 0.5 | 16 | >32 | >32 |
| 49 (4 mcg/ml) | | | | | | | |
| Ceftazidime + Example | 0.5 | 1 | 0.06 | 0.06 | 4 | 8 | 4 |
| 50 (4 mcg/ml) | | | | | | | |

1. A compound of Formula (I): 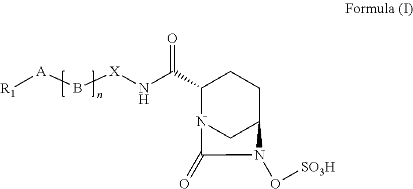
wherein:
A is a four to six membered nitrogen containing ring optionally substituted with one or more substituents selected from C₁-C₆ alkyl, oxo, NR₂R₃, aryl, heteroaryl, cycloalkyl or heterocycloalkyl;
B is C═O or CH—Z;
X is O or CONH;
Z is H, C₁-C₆ alkyl, OR₂ or NR₂R₃;
R₁ is selected from:
(a) hydrogen,
(b) C₁-C₆ alkyl optionally substituted with one or more substituents independently selected from OR₂, NR₂R₃, SR₂, halogen, CN, COOR₂, CONR₂R₃, SR₂, CH₂OR₂, CH₂NR₂R₃, OCOR₂, aryl, heteroaryl, cycloalkyl or heterocycloalkyl,
(c) CN,
(d) CONR₂R₃,
(e) COOR₂,
(f) NR₂R₃,
(g) NHCOOR₂,
(h) aryl,
(i) heteroaryl,
(j) cycloalkyl, or
(k) heterocycloalkyl;
R₂ and R₃ are each independently selected from:
(a) hydrogen,
(b) C₁-C₆ alkyl optionally substituted with one or more substituents selected from halogen, NR₄R₅, CONR₄R₅, CN, OR₄ or COOR₄;
(c) aryl,
(d) heteroaryl,
(e) cycloalkyl, or
(f) heterocycloalkyl;
R₄ and R₅ are each independently selected from:
(a) hydrogen or
(b) C₁-C₆ alkyl optionally substituted with one or more substituents selected from OH, halogen, NH₂, CONH₂, CN, OCH₃, or COOH;
n is 0, 1, 2, 3 or 4;
or a stereoisomer or a pharmaceutically acceptable salt thereof.

2. The compound of Formula (I) according to claim 1, wherein X is CONH₂.

3. The compound of Formula (I) according to claim 1, wherein X is O.

4. The compound of Formula (I) according to claim 1, wherein A is attached to B through nitrogen atom.

5. The compound of Formula (I) according to claim 1, wherein A is five membered nitrogen containing heteroaryl ring.

6. The compound of Formula (I) according to claim 1, wherein A is five membered nitrogen containing heteroaryl ring and A is attached to B through nitrogen atom.

7. The compound of Formula (I) according to claim 1, wherein X is CONH₂, A is five membered nitrogen containing heteroaryl ring and A is attached to B through nitrogen atom.

8. The compound of Formula (I) according to claim 1, wherein X is O, A is five membered nitrogen containing heteroaryl ring and A is attached to B through nitrogen atom.

9. The compound of Formula (I) according to claim 1, wherein A is tetrazole, pyrrole, imidazole or triazole.

10. The compound of Formula (I) according to claim 1, X is CONH₂, A is tetrazole, pyrrole, imidazole or triazole, and A is attached to B through nitrogen atom.

11. The compound of Formula (I) according to claim 1, wherein X is O, A is tetrazole, pyrrole, imidazole or triazole and A is attached to B through nitrogen atom.

12. The compound according to claim 1, selected from:
(2S,5R)-7-oxo-6-(sulfooxy)-N′-(H-tetrazol-5-ylcarbonyl)-1,6-diazabicyclo[3.2.1]octane-2-carbohydrazide;
(2S,5R)-N′-(1H-imidazol-1-yl-acetyl)-7-oxo-6-(sulfooxy)-1,6-diazabicyclo[3.2.1]octane-2-carbohydrazide;
(2S,5R)-7-oxo-6-(sulfooxy)-N′-(1H-tetrazol-1-ylacetyl)-1,6-diazabicyclo[3.2.1]octane-2-carbohydrazide;
(2S,5R)-7-oxo-6-(sulfooxy)-N′-(2H-tetrazol-2-ylacetyl)-1,6-diazabicyclo[3.2.1]octane-2-carbohydrazide;
(2S,5R)-N′-{[5-(ethoxycarbonyl)-1H-tetrazol-1-yl]acetyl}-7-oxo-6-(sulfooxy)-1,6-diazabicyclo[3.2.1]octane-2-carbohydrazide;
(2S,5R)-7-oxo-6-(sulfooxy)-N′-(2H-1,2,3-triazol-2-ylacetyl)-1,6-diazabicyclo[3.2.1]octane-2-carbohydrazide;
(2S,5R)-7-oxo-6-(sulfooxy)-N′-(1H-1,2,3-triazol-1-ylacetyl)-1,6-diazabicyclo[3.2.1]octane-2-carbohydrazide;
(2S,5R)-7-oxo-6-(sulfooxy)-N′-(1H-1,2,4-triazol-1-ylacetyl)-1,6-diazabicyclo[3.2.1]octane-2-carbohydrazide;
(2S,5R)-N′-{[5-methyl-1H-tetrazol-1-yl]acetyl}-7-oxo-6-(sulfooxy)-1,6-diazabicyclo[3.2.1]octane-2-carbohydrazide;
(2S,5R)-N′-{[4-methyl-1H-tetrazol-1-yl]acetyl}-7-oxo-6-(sulfooxy)-1,6-diazabicyclo[3.2.1]octane-2-carbohydrazide;
(2S,5R)-7-oxo-N′-(1H-pyrazol-1-ylacetyl)-6-(sulfooxy)-1,6-diazabicyclo[3.2.1]octane-2-carbohydrazide;
(2S,5R)-N′-[(5-amino-1H-tetrazol-1-yl)acetyl]-7-oxo-6-(sulfooxy)-1,6-diazabicyclo[3.2.1]octane-2-carbohydrazide;
(2S,5R)-N′-{[5-(aminomethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(aminomethyl)-2H-tetrazol-2-yl]acetyl}-7-oxo-6-(sulfooxy)-1,6-diazabicyclo[3.2.1]octane-2-carbohydrazide;
(2S,5R)-N′-[(3-amino-1H-1,2,4-triazol-1-yl)acetyl]-7-oxo-6-(sulfooxy)-1,6-diazabicyclo[3.2.1]octane-2-carbohydrazide;
(2S,5R)-7-oxo-6-(sulfooxy)-N′-(H-tetrazol-5-ylacetyl)-1,6-diazabicyclo[3.2.1]octane-2-carbohydrazide;
(2S,5R)-N′-[(5-carbamoyl-1H-tetrazol-1-yl)acetyl]-7-oxo-6-(sulfooxy)-1,6-diazabicyclo[3.2.1]octane-2-carbohydrazide;
(2S,5R)-N′-[(5-carbamoyl-2H-tetrazol-2-yl)acetyl]-7-oxo-6-(sulfooxy)-1,6-diazabicyclo[3.2.1]octane-2-carbohydrazide;
(2S,5R)-N′-[(5-cyano-1H-tetrazol-1-yl)acetyl]-7-oxo-6-(sulfooxy)-1,6-diazabicyclo[3.2.1]octane-2-carbohydrazide;
(2S,5R)-N′-[(5-cyano-2H-tetrazol-2-yl)acetyl]-7-oxo-6-(sulfooxy)-1,6-diazabicyclo[3.2.1]octane-2-carbohydrazide;
(2S,5R)-7-oxo-6-(sulfooxy)-N′-[(2S)-2-(1H-tetrazol-1-yl)propanoyl]-1,6-diazabicyclo[3.2.1]octane-2-carbohydrazide;
(2S,5R)-7-oxo-6-(sulfooxy)-N′-[(2R)-2-(1H-tetrazol-1-yl)propanoyl]-1,6-diazabicyclo[3.2.1]octane-2-carbohydrazide;
(2S,5R)-N′-{[5-(2-ethoxy-2-oxoethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(2-ethoxy-2-oxoethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(cyanomethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(cyanomethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(2-hydroxyethyl)-2H-tetrazol-1-yl]acetyl}-7-oxo-6-(sulfooxy)-1,6-diazabicyclo[3.2.1]octane-2-carbohydrazide;
(2S,5R)-N′-{[2-(2-aminoethyl)-2H-tetrazol-5-yl]acetyl}-7-oxo-6-(sulfooxy)-1,6-diazabicyclo[3.2.1]octane-2-carbohydrazide;
(2S,5R)-N′-{[1-(2-aminoethyl)-1H-tetrazol-5-yl]acetyl}-7-oxo-6-(sulfooxy)-1,6-diazabicyclo[3.2.1]octane-2-carbohydrazide;
(2S,5R)-N′-{[4-(aminomethyl)-2H-1,2,3-triazol-2-yl]acetyl}-7-oxo-6-(sulfooxy)-1,6-diazabicyclo[3.2.1]octane-2-carbohydrazide;
(2S,5R)-N′-{[4-(aminomethyl)-1H-1,2,3-triazol-1-yl]acetyl}-7-oxo-6-(sulfooxy)-1,6-diazabicyclo[3.2.1]octane-2-carbohydrazide;
(2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide;
(2S,5R)-N′-{[4-(1-methoxy-1-oxomethyl)-1H-1,2,3-triazol-1-yl]acetyl}-7-oxo-6-(sulfooxy)-1,6-diazabicyclo[3.2.1]octane-2-carbohydrazide;
(2S,5R)-N′-{[4-carbamoyl-1H-1,2,3-triazol-1-yl]acetyl}-7-oxo-6-(sulfooxy)-1,6-diazabicyclo[3.2.1]octane-2-carbohydrazide;
(2S,5R)-N′-[(4-cyano-1H-1,2,3-triazol-1-yl)acetyl]-7-oxo-6-(sulfooxy)-1,6-diazabicyclo[3.2.1]octane-2-carbohydrazide;
(2S,5R)-N′-{[5-(2-aminoethoxy)-1H-tetrazol-1-yl]acetyl}-7-oxo-6-(sulfooxy)-1,6-diazabicyclo[3.2.1]octane-2-carbohydrazide;
(2S,5R)-N′-{[5-(2-amino-1-oxo-ethoxy)-1H-tetrazol-1-yl]acetyl}-7-oxo-6-(sulfooxy)-1,6-diazabicyclo[3.2.1]octane-2-carbohydrazide;
(2S,5R)-N′-[(2-methyl-1H-imidazol-1-yl)acetyl]-7-oxo-6-(sulfooxy)-1,6-diazabicyclo[3.2.1]octane-2-carbohydrazide;
(2S,5R)-N′-[(2,4-dimethyl-1H-imidazol-1-yl)acetyl]-7-oxo-6-(sulfooxy)-1,6-diazabicyclo[3.2.1]octane-2-carbohydrazide;
(2S,5R)-N′-[(2,5-dimethyl-1H-imidazol-1-yl)acetyl]-7-oxo-6-(sulfooxy)-1,6-diazabicyclo[3.2.1]octane-2-carbohydrazide;
(2S,5R)-N′-{[2-(1-methoxy-1-oxomethyl)-1H-imidazol-1-yl]acetyl}-7-oxo-6-(sulfooxy)-1,6-diazabicyclo[3.2.1]octane-2-carbohydrazide;
(2S,5R)-N′-{[2-(1-methoxy-1-oxomethyl)-5-methyl-1H-imidazol-1-yl]acetyl}-7-oxo-6-(sulfooxy)-1,6-diazabicyclo[3.2.1]octane-2-carbohydrazide;
(2S,5R)-N′-{[2-phenyl-5-methyl-1H-imidazol-1-yl]acetyl}-7-oxo-6-(sulfooxy)-1,6-diazabicyclo[3.2.1]octane-2-carbohydrazide;
(2S,5R)-N′-(1H-benzimidazol-1-ylacetyl)-7-oxo-6-(sulfooxy)-1,6-diazabicyclo[3.2.1]octane-2-carbohydrazide;
(2S,5R)-N′-{[5-(2-aminoethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(2-aminoethyl)-1H-tetrazol-1-yl]acetyl}-7-oxo-6-(sulfooxy)-1,6-diazabicyclo[3.2.1]octane-2-carbohydrazide;
(2S,5R)-N′-{[543-aminopropyl)-2H-tetrazol-2-yl]acetyl}-7-oxo-6-(sulfooxy)-1,6-diazabicyclo[3.2.1]octane-2-carbohydrazide;
(2S,5R)-N′-{[5-(3-aminopropyl)-1H-tetrazol-1-yl]acetyl}-7-oxo-6-(sulfooxy)-1,6-diazabicyclo[3.2.1]octane-2-carbohydrazide;
(2S,5R)-N′-{[5-(4-aminobutyl)-2H-tetrazol-2-yl]acetyl}-7-oxo-6-(sulfooxy)-1,6-diazabicyclo[3.2.1]octane-2-carbohydrazide;
(2S,5R)-N′-{[5-(4-aminobutyl)-1H-tetrazol-1-yl]acetyl}-7-oxo-6-(sulfooxy)-1,6-diazabicyclo[3.2.1]octane-2-carbohydrazide;
(2S,5R)-N′-[(2-methyl-1H-imidazol-1-yl)acetyl]-7-oxo-6-(sulfooxy)-1,6-diazabicyclo[3.2.1]octane-2-carbohydrazide;
(2S,5R)-N′-[(2S)-2-(1H-imidazol-1-yl)propanoyl]-7-oxo-6-(sulfooxy)-1,6-diazabicyclo[3.2.1]octane-2-carbohydrazide;
(2S,5R)-N′-{[2-(1[(2S)-pyrrolidin-2-ylcarbonyl]oxy}methyl)-1H-imidazol-1-yl]acetyl}-7-oxo-6-(sulfooxy)-1,6-diazabicyclo[3.2.1]octane-2-carbohydrazide;
(2S,5R)-N′-{[2-({[(2S)-2-aminopropanoyl]oxy}methyl)-1H-imidazol-1-yl]acetyl}-7-oxo-6-(sulfooxy)-1,6-diazabicyclo[3.2.1]octane-2-carbohydrazide;
(2S,5R)-N′-{[5-(2-oxo-2-oxyethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(2-oxo-2-oxyethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide; (2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;
(2S,5R)-N′-{[5-(carbamoyl)-2H-tetrazol-2-yl]acetyl}-7-oxo-6-(sulfooxy)-1,6-diazabicyclo[3.2.1]octane-2-carbohydrazide;
(2S,5R)-N′-{[5-(carbamoyl)-1H-tetrazol-1-yl]acetyl}-7-oxo-6-(sulfooxy)-1,6-diazabicyclo[3.2.1]octane-2-carbohydrazide;
(2S,5R)-7-oxo-N′-(1H-pyrrol-1-ylacetyl)-6-(sulfooxy)-1,6-diazabicyclo[3.2.1]octane-2-carbohydrazide;
(2S,5R)-N′-{[5-(2-hydroxyethyl)-2H-tetrazol-2-yl]acetyl}-7-oxo-6-(sulfooxy)-1,6-diazabicyclo[3.2.1]octane-2-carbohydrazide;
(2S,5R)-N′-{[5-(2-hydroxyethyl)-1H-tetrazol-l-yl]acetyl}-7-oxo-6-(sulfooxy)-1,6-diazabicyclo[3.2.1]octane-2-carbohydrazide;
(2S,5R)-N′-{[2-(2-hydroxyethyl)-1H-imidazol-1-yl]acetyl}-7-oxo-6-(sulfooxy)-1,6-diazabicyclo[3.2.1]octane-2-carbohydrazide;
(2S, 5R)-N′-{[4-amino-1H-imidazol-1-yl]-acetyl}-7-oxo-6-(sulfooxy)-1,6-diazabicyclo[3.2.1]octane-2-carbohydrazide;
(2S, 5R)-N′-{[4-amino-2-oxopyrimidin-1(2H)-yl]acetyl}-7-oxo-6-(sulfooxy)-1,6-diazabicyclo[3.2.1]octane-2-carbohydrazide;
(2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide;
Mixture of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide and (2S,5R)-N′-{[5-(hydroxymethyl)-1H-1,2,3-triazol-1-yl]acetyl}-7-oxo-6-(sulfooxy)-1,6-diazabicyclo[3.2.1]octane-2-carbohydrazide;
(2S,5R)-N′-{[2-(hydroxymethyl)-1H-imidazol-1-yl]acetyl}-7-oxo-6-(sulfooxy)-1,6-diazabicyclo[3.2.1]octane-2-carbohydrazide;
(2S,5R)-N′-[2-(ethoxycarbonyl)-1H-imidazol-1-ylacetyl]-7-oxo-6-(sulfooxy)-1,6-diazabicyclo[3.2.1]octane-2-carbohydrazide;
(2S,5R)-N′-[2-(carbamoyl)-1H-imidazol-1-ylacetyl]-7-oxo-6-(sulfooxy)-1,6-diazabicyclo[3.2.1]octane-2-carbohydrazide;
(2S,5R)-N′-[2-(methoxycarbonyl)-1H-imidazol-1-ylacetyl]-7-oxo-6-(sulfooxy)-1,6-diazabicyclo[3.2.1]octane-2-carbohydrazide;
(2S,5R)-N-{2-[5-(aminomethyl)-2H-tetrazol-2-yl]ethoxy}-7-oxo-6-(sulfooxy)-1,6-diazabicyclo[3.2.1]octane-2-carboxamide;
(2S,5R)-N-(2-(2H-1,2,3-triazol-2-yl)ethoxy)-6-(sulfooxy)-7-oxo-1,6-diaza-bicyclo[3.2.1]octane-2-carboxamide;
(2S,5R)-N-(2-(1H-1,2,3-triazol-1-yl)ethoxy)-6-(sulfooxy)-7-oxo-1,6-diaza-bicyclo[3.2.1]octane-2-carboxamide;
(2S,5R)-7-oxo-6-(sulfooxy)-N-[2-(1H-1,2,4-triazol-1-yl)ethoxy]-1,6-diazabicyclo[3.2.1]octane-2-carboxamide;
(2S,5R)-N-{2-[5-(ethoxycarbonyl)-1H-tetrazol-1-yl]ethoxy}-6-sulfooxy-7-oxo-1,6-diazabicyclo[3.2.1]octane-2-carboxamide;
(2S,5R)-7-oxo-6-(sulfooxy)-N-[2-(2H-tetrazol-2-yl)ethoxy]-1,6-diazabicyclo[3.2.1]octane-2-carboxamide;
(2S,5R)-7-oxo-6-(sulfooxy)-N-[2-(1H-tetrazol-1-yl)ethoxy]-1,6-diazabicyclo[3.2.1]octane-2-carboxamide;
(2S,5R)-N-[2-(5-methyl-1H-tetrazol-1-yl)ethoxy]-7-oxo-6-(sulfooxy)-1,6-diazabicyclo[3.2.1]octane-2-carboxamide;
(2S,5R)-N-[2-(5-methyl-2H-tetrazol-2-yl)ethoxy]-7-oxo-6-(sulfooxy)-1,6-diazabicyclo[3.2.1]octane-2-carboxamide;
(2S,5R)-N-[2-(3-amino-1H-1,2,4-triazol-1-yl)ethoxy]-7-oxo-6-(sulfooxy)-1,6-diazabicyclo[3.2.1]octane-2-carboxamide;
(2S,5R)-N-[2-(5-amino-1H-tetrazol-1-yl)ethoxy]-7-oxo-6-(sulfooxy)-1,6-diazabicyclo[3.2.1]octane-2-carboxamide;
(2S,5R)-N-[2-(5-amino-2H-tetrazol-2-yl)ethoxy]-7-oxo-6-(sulfooxy)-1,6-diazabicyclo[3.2.1]octane-2-carboxamide;
(2S,5R)-N-[2-(5-carbamoyl-1H-tetrazol-1-yl)ethoxy]-7-oxo-6-(sulfooxy)-1,6-diazabicyclo[3.2.1]octane-2-carboxamide;
(2S,5R)-N-{2-[5-(2-ethoxy-2-oxoethyl)-2H-tetrazol-2-yl]ethoxy}-6-sulfooxy-7-oxo-1,6-diazabicyclo[3.2.1]octane-2-carboxamide;
(2S,5R)-N-{2-[5-(2-ethoxy-2-oxoethyl)-1H-tetrazol-1-yl]ethoxy}-6-(sulfooxy)-7-oxo-1,6-diazabicyclo[3.2.1]octane-2-carboxamide;
(2S,5R)-N-{2-[5-(2-amino-2-oxoethyl)-2H-tetrazol-2-yl]ethoxy}-7-oxo-6-(sulfooxy)-1,6-diazabicyclo[3.2.1]octane-2-carboxamide;
(2S,5R)-N-{2-[5-(2-amino-2-oxoethyl)-1H-tetrazol-1-yl]ethoxy}-7-oxo-6-(sulfooxy)-1,6-diazabicyclo[3.2.1]octane-2-carboxamide;
(2S,5R)-N-{2-[5-(aminomethoxy)-1H-tetrazol-1-yl]ethoxy}-7-oxo-6-(sulfooxy)-1,6-diazabicyclo[3.2.1]octane-2-carboxamide;
(2S,5R)-N-{2-[5-(2-aminoethoxy)-1H-tetrazol-1-yl]ethoxy}-7-oxo-6-(sulfooxy)-1,6-diazabicyclo[3.2.1]octane-2-carboxamide;
(2S,5R)-N-{2-[5-(3-aminopropoxy)-1H-tetrazol-1-yl]ethoxy}-6-(sulfooxy)-7-oxo-1,6-diazabicyclo[3.2.1]octane-2-carboxamide;
(2S,5R)-N-{2-[5-(2-amino-1-oxo-ethoxy)-1H-tetrazol-1-yl]ethoxy}-7-oxo-6-(sulfooxy)-1,6-diazabicyclo[3.2.1]octane-2-carboxamide;
(2S,5R)-N-{2-[5-(2-aminoethyl)-2H-tetrazol-2-yl]ethoxy}-7-oxo-6-(sulfooxy)-1,6-diazabicyclo[3.2.1]octane-2-carboxamide;
(2S,5R)-N-{2-[4-(hydroxymethyl)-2H-1,2,3-triazol-2-yl]ethoxy}-7-oxo-6-(sulfooxy)-1,6-diazabicyclo[3.2.1]octane-2-carboxamide;
(2S,5R)-N-{2-[5-(2-hydroxyethyl)-2H-tetrazol-2-yl]ethoxy}-7-oxo-6-(sulfooxy)-1,6-diazabicyclo[3.2.1]octane-2-carboxamide;
(2S,5R)-N-{2-[5-(2-hydroxyethyl)-1H-tetrazol-1-yl]ethoxy}-7-oxo-6-(sulfooxy)-1,6-diazabicyclo[3.2.1]octane-2-carboxamide;
(2S,5R)-N-{2-[5-(2-oxo-2-ethoxyethyl)-2H-tetrazol-2-yl]ethoxy}-7-oxo-6-(sulfooxy)-1,6-diazabicyclo[3.2.1]octane-2-carboxamide;
or a stereoisomer or a pharmaceutically acceptable salt thereof.

13. The compound according to claim 1, selected from:
Di sodium salt of (2S,5R)-7-oxo-6-(sulfooxy)-N′-(H-tetrazol-5-ylcarbonyl)-1,6-diazabicyclo[3.2.1]octane-2-carbohydrazide;
Sodium salt of (2S,5R)-N′-(1H-imidazol-1-yl-acetyl)-7-oxo-6-(sulfooxy)-1,6-diazabicyclo[3.2.1]octane-2-carbohydrazide;
Sodium salt of (2S,5R)-7-oxo-6-(sulfooxy)-N′-(1H-tetrazol-1-ylacetyl)-1,6-diazabicyclo[3.2.1]octane-2-carbohydrazide;
Sodium salt of (2S,5R)-7-oxo-6-(sulfooxy)-N′-(2H-tetrazol-2-ylacetyl)-1,6-diazabicyclo[3.2.1]octane-2-carbohydrazide;
Sodium salt of (2S,5R)-N′-{[5-(ethoxycarbonyl)-1H-tetrazol-1-yl]acetyl}-7-oxo-6-(sulfooxy)-1,6-diazabicyclo[3.2.1]octane-2-carbohydrazide;
Sodium salt of (2S,5R)-7-oxo-6-(sulfooxy)-N′-(2H-1,2,3-triazol-2-ylacetyl)-1,6-diazabicyclo[3.2.1]octane-2-carbohydrazide;
Sodium salt of (2S,5R)-7-oxo-6-(sulfooxy)-N′-(1H-1,2,3-triazol-1-ylacetyl)-1,6-diazabicyclo[3.2.1]octane-2-carbohydrazide;
Sodium salt of (2S,5R)-7-oxo-6-(sulfooxy)-N′-(1H-1,2,4-triazol-1-ylacetyl)-1,6-diazabicyclo[3.2.1]octane-2-carbohydrazide;
Sodium salt of (2S,5R)-N′-{[5-methyl-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[4-methyl-1H-tetrazol-1-yl]acetyl}-7-oxo-6-(sulfooxy)-1,6-diazabicyclo[3.2.1]octane-2-carbohydrazide;
Sodium salt of (2S,5R)-7-oxo-N′-(1H-pyrazol-1-ylacetyl)-6-(sulfooxy)-1,6-diazabicyclo[3.2.1]octane-2-carbohydrazide;
Sodium salt of (2S,5R)-N′-[(5-amino-1H-tetrazol-1-yl)acetyl]-7-oxo-6-(sulfooxy)-1,6-diazabicyclo[3.2.1]octane-2-carbohydrazide;
Sodium salt of (2S,5R)-N′-{[5-(aminomethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(aminomethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-[(3-amino-1H-1,2,4-triazol-1-yl)acetyl]-7-oxo-6-(sulfooxy)-1,6-diazabicyclo[3.2.1]octane-2-carbohydrazide;
Disodium salt of (2S,5R)-7-oxo-6-(sulfooxy)-N′-(H-tetrazol-5-ylacetyl)-1,6-diazabicyclo[3.2.1]octane-2-carbohydrazide;
Sodium salt of (2S,5R)-N′-[(5-carbamoyl-1H-tetrazol-1-yl)acetyl]-7-oxo-6-(sulfooxy)-1,6-diazabicyclo[3.2.1]octane-2-carbohydrazide;
Sodium salt of (2S,5R)-N′-[(5-carbamoyl-2H-tetrazol-2-yl)acetyl]-7-oxo-6-(sulfooxy)-1,6-diazabicyclo[3.2.1]octane-2-carbohydrazide;
Sodium salt of (2S,5R)-N′-[(5-cyano-1H-tetrazol-1-yl)acetyl]-7-oxo-6-(sulfooxy)-1,6-diazabicyclo[3.2.1]octane-2-carbohydrazide;
Sodium salt of (2S,5R)-N′-[(5-cyano-2H-tetrazol-2-yl)acetyl]-7-oxo-6-(sulfooxy)-1,6-diazabicyclo[3.2.1]octane-2-carbohydrazide;
Sodium salt of (2S,5R)-7-oxo-6-(sulfooxy)-N′-[(2S)-2-(1H-tetrazol-1-yl)propanoyl]-1,6-diazabicyclo[3.2.1]octane-2-carbohydrazide;
Sodium salt of (2S,5R)-7-oxo-6-(sulfooxy)-N′-[(2R)-2-(1H-tetrazol-1-yl)propanoyl]-1,6-diazabicyclo[3.2.1]octane-2-carbohydrazide;
Sodium salt of (2S,5R)-N′-{[5-(2-ethoxy-2-oxoethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(2-ethoxy-2-oxoethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5(2-amino-2-oxoethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(cyanomethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(cyanomethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(2-hydroxyethyl)-2H-tetrazol-1-yl]acetyl}-7-oxo-6-(sulfooxy)-1,6-diazabicyclo[3.2.1]octane-2-carbohydrazide;
Sodium salt of (2S,5R)-N′-{[2-(2-aminoethyl)-2H-tetrazol-5-yl]acetyl}-7-oxo-6-(sulfooxy)-1,6-diazabicyclo[3.2.1]octane-2-carbohydrazide;
Sodium salt of (2S,5R)-N′-{[1-(2-aminoethyl)-1H-tetrazol-5-yl]acetyl}-7-oxo-6-(sulfooxy)-1,6-diazabicyclo[3.2.1]octane-2-carbohydrazide;
Sodium salt of (2S,5R)-N′-{[4-(aminomethyl)-2H-1,2,3-triazol-2-yl]acetyl}-7-oxo-6-(sulfooxy)-1,6-diazabicyclo[3.2.1]octane-2-carbohydrazide;
Sodium salt of (2S,5R)-N′-{[4-(aminomethyl)-1H-1,2,3-triazol-1-yl]acetyl}-7-oxo-6-(sulfooxy)-1,6-diazabicyclo[3.2.1]octane-2-carbohydrazide;
Sodium salt of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide;
Sodium salt of (2S,5R)-N′-{[4-(1-methoxy-1-oxomethyl)-1H-1,2,3-triazol-1-yl]acetyl}-7-oxo-6-(sulfooxy)-1,6-diazabicyclo[3.2.1]octane-2-carbohydrazide;
Sodium salt of (2S,5R)-N′-{[4-carbamoyl-1H-1,2,3-triazol-1-yl]acetyl}-7-oxo-6-(sulfooxy)-1,6-diazabicyclo[3.2.1]octane-2-carbohydrazide;
Sodium salt of (2S,5R)-N′-[(4-cyano-1H-1,2,3-triazol-1-yl)acetyl]-7-oxo-6-(sulfooxy)-1,6-diazabicyclo[3.2.1]octane-2-carbohydrazide;
Sodium salt of (2S,5R)-N′-{[5-(2-aminoethoxy)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(2-amino-1-oxo-ethoxy)-1H-tetrazol-1-yl]acetyl}-7-oxo-6-(sulfooxy)-1,6-diazabicyclo[3.2.1]octane-2-carbohydrazide;
Sodium salt of (2S,5R)-N′-[(2-methyl-1H-imidazol-1-yl)acetyl]-7-oxo-6-(sulfooxy)-1,6-diazabicyclo[3.2.1]octane-2-carbohydrazide;
Sodium salt of (2S,5R)-N′-[(2,4-dimethyl-1H-imidazol-1-yl)acetyl]-7-oxo-6-(sulfooxy)-1,6-diazabicyclo[3.2.1]octane-2-carbohydrazide;
Sodium salt of (2S,5R)-N′-[(2,5-dimethyl-1H-imidazol-1-yl)acetyl]-7-oxo-6-(sulfooxy)-1,6-diazabicyclo[3.2.1]octane-2-carbohydrazide;
Sodium salt of (2S,5R)-N′-{[2-(1-methoxy-1-oxomethyl)-1H-imidazol-1-yl]acetyl}-7-oxo-6-(sulfooxy)-1,6-diazabicyclo[3.2.1]octane-2-carbohydrazide;
Sodium salt of (2S,5R)-N′-{[2-(1-methoxy-1-oxomethyl)-5-methyl-1H-imidazol-1-yl]acetyl}-7-oxo-6-(sulfooxy)-1,6-diazabicyclo[3.2.1]octane-2-carbohydrazide;
Sodium salt of (2S,5R)-N′-{[2-phenyl-5-methyl-1H-imidazol-1-yl]acetyl}-7-oxo-6-(sulfooxy)-1,6-diazabicyclo[3.2.1]octane-2-carbohydrazide;
Sodium salt of (2S,5R)-N′-(1H-benzimidazol-1-ylacetyl)-7-oxo-6-(sulfooxy)-1,6-diazabicyclo[3.2.1]octane-2-carbohydrazide;
Sodium salt of (2S,5R)-N′-{[5-(2-aminoethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-aminoethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(3-aminopropyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(3-aminopropyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(4-amino butyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(4-aminobutyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-[(2-methyl-1H-imidazol-1-yl)acetyl]-7-oxo-6-(sulfooxy)-1,6-diazabicyclo[3.2.1]octane-2-carbohydrazide;
Sodium salt of (2S,5R)-N′-[(2S)-2-(1H-imidazol-1-yl)propanoyl]-7-oxo-6-(sulfooxy)-1,6-diazabicyclo[3.2.1]octane-2-carbohydrazide;
Sodium salt of (2S,5R)-N′-{[2-({[(2S)-pyrrolidin-2-ylcarbonyl]oxy}methyl)-1H-imidazol-1-yl]acetyl}-7-oxo-6-(sulfooxy)-1,6-diazabicyclo[3.2.1]octane-2-carbohydrazide;
Sodium salt of (2S,5R)-N′-{[2-({[(2S)-2-aminopropanoyl]oxy}methyl)-1H-imidazol-1-yl]acetyl}-7-oxo-6-(sulfooxy)-1,6-diazabicyclo[3.2.1]octane-2-carbohydrazide;
Sodium salt of (2S,5R)-N′-{[5-(2-oxo-2-oxyethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(2-oxo-2-oxyethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-amino-2-oxoethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-amino-2-oxoethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[5-(carbamoyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(carbamoyl)-1H-tetrazol-1-yl]acetyl}-7-oxo-6-(sulfooxy)-1,6-diazabicyclo[3.2.1]octane-2-carbohydrazide;
Sodium salt of (2S,5R)-7-oxo-N-(1H-pyrrol-1-ylacetyl)-6-(sulfooxy)-1,6-diazabicyclo[3.2.1]octane-2-carbohydrazide;
Sodium salt of (2S,5R)-N′-{[5-(2-hydroxyethyl)-2H-tetrazol-2-yl]acetyl}-7-oxo-6-(sulfooxy)-1,6-diazabicyclo[3.2.1]octane-2-carbohydrazide;
Sodium salt of (2S,5R)-N′-{[5-(2-hydroxyethyl)-1H-tetrazol-1-yl]acetyl}-7-oxo-6-(sulfooxy)-1,6-diazabicyclo[3.2.1]octane-2-carbohydrazide;
Sodium salt of (2S,5R)-N′-{[2-(2-hydroxyethyl)-1H-imidazol-1-yl]acetyl}-7-oxo-6-(sulfooxy)-1,6-diazabicyclo[3.2.1]octane-2-carbohydrazide;
Sodium and Trifluoroacetic acid salt of (2S, 5R)-N-{[4-amino-1H-imidazol-1-yl]-acetyl}-7-oxo-6-(sulfooxy)-1,6-diazabicyclo[3.2.1]octane-2-carbohydrazide;
Sodium and Trifluoroacetic acid salt of (2S, 5R)-N-{[4-amino-2-oxopyrimidin-1(2H)-yl]acetyl}-7-oxo-6-(sulfooxy)-1,6-diazabicyclo[3.2.1]octane-2-carbohydrazide;
Sodium salt of (2S,5R)-N′-{[4-(hydroxymethyl)-2H-1,2,3-triazol-2-yl]acetyl}-7-oxo-6-(sulfooxy)-1,6-diazabicyclo[3.2.1]octane-2-carbohydrazide;
Mixture of sodium salt of (2S,5R)-N′-{[4-(hydroxymethyl)-1H-1,2,3-triazol-1-yl]acetyl}-7-oxo-6-(sulfooxy)-1,6-diazabicyclo[3.2.1]octane-2-carbohydrazide and (2S,5R)-N′-{[5-(Hydroxymethyl)-1H-1,2,3-triazol-1-yl]acetyl}-7-oxo-6-(sulfooxy)-1,6-diazabicyclo[3.2.1]octane-2-carbohydrazide;
Sodium salt of (2S,5R)-N′-{[2-(hydroxymethyl)-1H-imidazol-1-yl]acetyl}-7-oxo-6-(sulfooxy)-1,6-diazabicyclo[3.2.1]octane-2-carbohydrazide;
Sodium salt of (2S,5R)-N′-[2-(ethoxycarbonyl)-1H-imidazol-1-ylacetyl]-7-oxo-6-(sulfooxy)-1,6-diazabicyclo[3.2.1]octane-2-carbohydrazide;
Sodium salt of (2S,5R)-N′-[2-(carbamoyl)-1H-imidazol-1-ylacetyl]-7-oxo-6-(sulfooxy)-1,6-diazabicyclo[3.2.1]octane-2-carbohydrazide;
Sodium salt of (2S,5R)-N′-[2-(methoxycarbonyl)-1H-imidazol-1-ylacetyl]-7-oxo-6-(sulfooxy)-1,6-diazabicyclo[3.2.1]octane-2-carbohydrazide;
Sodium salt of (2S,5R)-N-{2-[5-(aminomethyl)-2H-tetrazol-2-yl]ethoxy}-7-oxo-6-(sulfooxy)-1,6-diazabicyclo[3.2.1]octane-2-carboxamide;
Sodium salt of (2S,5R)-N-(2-(2H-1,2,3-triazol-2-yl)ethoxy)-6-(sulfooxy)-7-oxo-1,6-diaza-bicyclo[3.2.1]octane-2-carboxamide.
Sodium salt of (2S,5R)-N-(2-(1H-1,2,3-triazol-1-yl)ethoxy)-6-(sulfooxy)-7-oxo-1,6-diaza-bicyclo[3.2.1]octane-2-carboxamide;
Sodium salt of (2S,5R)-7-oxo-6-(sulfooxy)-N-[2-(1H-1,2,4-triazol-1-yl)ethoxy]-1,6-diazabicyclo[3.2.1]octane-2-carboxamide;
Sodium salt of (2S,5R)-N-{2-[5-(ethoxycarbonyl)-1H-tetrazol-1-yl]ethoxy}-6-sulfooxy-7-oxo-1,6-diazabicyclo[3.2.1]octane-2-carboxamide;
Sodium salt of (2S,5R)-7-oxo-6-(sulfooxy)-N-[2-(2H-tetrazol-2-yl)ethoxy]-1,6-diazabicyclo[3.2.1]octane-2-carboxamide;
Sodium salt of (2S,5R)-7-oxo-6-(sulfooxy)-N-[2-(1H-tetrazol-1-yl)ethoxy]-1,6-diazabicyclo[3.2.1]octane-2-carboxamide;
Sodium salt of (2S,5R)-N-[2-(5-methyl-1H-tetrazol-1-yl)ethoxy]-7-oxo-6-(sulfooxy)-1,6-diazabicyclo[3.2.1] octane-2-carboxamide;
Sodium salt of (2S,5R)-N-[2-(5-methyl-2H-tetrazol-2-yl)ethoxy]-7-oxo-6-(sulfooxy)-1,6-diazabicyclo[3.2.1]octane-2-carboxamide;
Sodium salt of (2S,5R)-N-[2-(3-amino-1H-1,2,4-triazol-1-yl)ethoxy]-7-oxo-6-(sulfooxy)-1,6-diazabicyclo[3.2.1]octane-2-carboxamide;
Sodium salt of (2S,5R)-N-[2-(5-amino-1H-tetrazol-1-yl)ethoxy]-7-oxo-6-(sulfooxy)-1,6-diazabicyclo[3.2.1]octane-2-carboxamide;
Sodium salt of (2S,5R)-N-[2-(5-amino-2H-tetrazol-2-yl)ethoxy]-7-oxo-6-(sulfooxy)-1,6-diazabicyclo[3.2.1]octane-2-carboxamide;
Sodium salt of (2S,5R)-N-[2-(5-carbamoyl-1H-tetrazol-1-yl)ethoxy]-7-oxo-6-(sulfooxy)-1,6-diazabicyclo[3.2.1]octane-2-carboxamide;
Sodium salt of (2S,5R)-N-{2-[5-(2-ethoxy-2-oxoethyl)-2H-tetrazol-2-yl]ethoxy}-6-sulfooxy-7-oxo-1,6-diazabicyclo[3.2.1]octane-2-carboxamide;
Sodium salt of (2S,5R)-N-{2-[5-(2-ethoxy-2-oxoethyl)-1H-tetrazol-1-yl]ethoxy}-6-(sulfooxy)-7-oxo-1,6-diazabicyclo[3.2.1]octane-2-carboxamide;
Sodium salt of (2S,5R)-N-{2-[5-(2-amino-2-oxoethyl)-2H-tetrazol-2-yl]ethoxy}-7-oxo-6-(sulfooxy)-1,6-diazabicyclo[3.2.1]octane-2-carboxamide;
Sodium salt of (2S,5R)-N-{2-[5-(2-amino-2-oxoethyl)-1H-tetrazol-1-yl]ethoxy}-7-oxo-6-(sulfooxy)-1,6-diazabicyclo[3.2.1]octane-2-carboxamide;
Sodium salt of (2S,5R)-N-{2-[5-(aminomethoxy)-1H-tetrazol-1-yl]ethoxy}-7-oxo-6-(sulfooxy)-1,6-diazabicyclo[3.2.1]octane-2-carboxamide;
Sodium salt of (2S,5R)-N-{2-[5-(2-aminoethoxy)-1H-tetrazol-1-yl]ethoxy}-7-oxo-6-(sulfooxy)-1,6-diazabicyclo[3.2.1]octane-2-carboxamide;
Sodium salt of (2S,5R)-N-{2-[5-(3-aminopropoxy)-1H-tetrazol-1-yl]ethoxy}-6-(sulfooxy)-7-oxo-1,6-diazabicyclo[3.2.1]octane-2-carboxamide;
Sodium salt of (2S,5R)-N-{2-[5-(2-amino-1-oxo-ethoxy)-1H-tetrazol-1-yl]ethoxy}-7-oxo-6-(sulfooxy)-1,6-diazabicyclo[3.2.1]octane-2-carboxamide;
Sodium salt of (2S,5R)-N-{2-[5-(2-aminoethyl)-2H-tetrazol-2-yl]ethoxy}-7-oxo-6-(sulfooxy)-1,6-diazabicyclo[3.2.1]octane-2-carboxamide;
Sodium salt of (2S,5R)-N-{2-[4-(hydroxymethyl)-2H-1,2,3-triazol-2-yl]ethoxy}-7-oxo-6-(sulfooxy)-1,6-diazabicyclo[3.2.1]octane-2-carboxamide;
Sodium salt of (2S,5R)-N-{2-[5-(2-hydroxyethyl)-2H-tetrazol-2-yl]ethoxy}-7-oxo-6-(sulfooxy)-1,6-diazabicyclo[3.2.1]octane-2-carboxamide;
Sodium salt of (2S,5R)-N-{2-[5-(2-hydroxyethyl)-1H-tetrazol-1-yl]ethoxy}-7-oxo-6-(sulfooxy)-1,6-diazabicyclo[3.2.1]octane-2-carboxamide;
Sodium salt of (2S,5R)-N-{2-[5-(2-oxo-2-ethoxyethyl)-2H-tetrazol-2-yl]ethoxy}-7-oxo-6-(sulfooxy)-1,6-diazabicyclo[3.2.1]octane-2-carboxamide;
or a stereoisomer thereof.

14. A pharmaceutical composition comprising a compound according to any one of claims 1 to 13.

15. The pharmaceutical composition according to claim 14, further comprising at least one antibacterial agent.

16. The pharmaceutical composition according to claim 15, wherein the antibacterial agent is selected from a group consisting of aminoglycosides, ansamycins, penems, carbapenems, carbacephems, cephalosporins, cephamycins, lincosamides, lipopeptides, macrolides, monobactams, nitrofurans, penicillins, polypeptides, quinolones, sulfonamides, tetracyclines, or oxazolidinone antibacterial agents.

17. The pharmaceutical composition according to claim 15, wherein the antibacterial agent is a beta-lactam antibacterial agent.

18. The pharmaceutical composition according to claim 15, wherein the antibacterial agent is selected from a group consisting of cephalothin, cephaloridine, cefaclor, cefadroxil, cefamandole, cefazolin, cephalexin, cephradine, ceftizoxime, cefoxitin, cephacetrile, cefotiam, cefotaxime, cefsulodin, cefoperazone, ceftizoxime, cefmenoxime, cefmetazole, cephaloglycin, cefonicid, cefodizime, cefpirome, ceftazidime, ceftriaxone, cefpiramide, cefbuperazone, cefozopran, cefepime, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil or cefditoren pivoxil, cefuroxime, cefuroxime axetil, loracarbacef, ceftaroline, ceftolozane, latamoxef, piperacillin, imipenem, doripenem, and meropenem.

19. A method for treating a bacterial infection in a subject, the method comprising administering to the subject a pharmaceutically effective amount of a compound according to any one of claims 1 to 13.

20. A method for treating a bacterial infection in a subject, the method comprising administering to the subject a pharmaceutically effective amount of a pharmaceutical composition according to any one of claims 14 to 18.
